SEQUENCE LISTING (56 sequences)

SEQ ID NO: 1 - DNA, 24 bp, synthetic construct
  misc_feature 1..24: note = Synthetic Polynucleotide
  source 1..24: mol_type = other DNA
ggcctcagagaggacatagtgccc

SEQ ID NO: 2 - DNA, 20 bp, synthetic construct
  misc_feature 1..20: note = Synthetic Polynucleotide
  source 1..20: mol_type = other DNA
gccctctggtgtcaggtccc

SEQ ID NO: 3 - DNA, 22 bp, synthetic construct
  misc_feature 1..22: note = Synthetic Polynucleotide
  source 1..22: mol_type = other DNA
cctcacgcagcttaatgtggcc

SEQ ID NO: 4 - DNA, 28 bp, synthetic construct
  misc_feature 1..28: note = Synthetic Polynucleotide
  source 1..28: mol_type = other DNA
ggaagttaaggacgtgattacagggagc

SEQ ID NO: 5 - DNA, 20 bp, synthetic construct
  misc_feature 1..20: note = Synthetic Polynucleotide
  source 1..20: mol_type = other DNA
ctgggttgcatactggtggg

SEQ ID NO: 6 - DNA, 20 bp, synthetic construct
  misc_feature 1..20: note = Synthetic Polynucleotide
  source 1..20: mol_type = other DNA
aaacagggtctttgctgctg

SEQ ID NO: 7 - DNA, 20 bp, synthetic construct
  misc_feature 1..20: note = Synthetic Polynucleotide
  source 1..20: mol_type = other DNA
acaaaggtgtggcaagggtt

SEQ ID NO: 8 - DNA, 20 bp, synthetic construct
  misc_feature 1..20: note = Synthetic Polynucleotide
  source 1..20: mol_type = other DNA
ccaccggaagctacgacaaa

SEQ ID NO: 9 - DNA, 20 bp, synthetic construct
  misc_feature 1..20: note = Synthetic Polynucleotide
  source 1..20: mol_type = other DNA
gggggaattggaggcattct

SEQ ID NO: 10 - DNA, 20 bp, synthetic construct
  misc_feature 1..20: note = Synthetic Polynucleotide
  source 1..20: mol_type = other DNA
cttcttcccgtgctaccctc

SEQ ID NO: 11 - DNA, 21 bp, synthetic construct
  misc_feature 1..21: note = Synthetic Polynucleotide
  source 1..21: mol_type = other DNA
cctcccacctcttcgttatcc

SEQ ID NO: 12 - DNA, 20 bp, synthetic construct
  misc_feature 1..20: note = Synthetic Polynucleotide
  source 1..20: mol_type = other DNA
agtctgaggggcttttgcta

SEQ ID NO: 13 - DNA, 20 bp, synthetic construct
  misc_feature 1..20: note = Synthetic Polynucleotide
  source 1..20: mol_type = other DNA
agtctgaggggcttttgcta

SEQ ID NO: 14 - DNA, 25 bp, synthetic construct
  misc_feature 1..25: note = Synthetic Polynucleotide
  source 1..25: mol_type = other DNA
ggccccacctgtcaaagagagcagc

SEQ ID NO: 15 - DNA, 23 bp, synthetic construct
  misc_feature 1..23: note = Synthetic Polynucleotide
  source 1..23: mol_type = other DNA
caaacttgacacccgtgaggtcc

SEQ ID NO: 16 - DNA, 23 bp, synthetic construct
  misc_feature 1..23: note = Synthetic Polynucleotide
  source 1..23: mol_type = other DNA
tctatggctcccagacgggaacc

SEQ ID NO: 17 - DNA, 22 bp, synthetic construct
  misc_feature 1..22: note = Synthetic Polynucleotide
  source 1..22: mol_type = other DNA
ccaatcatagaagtcctgcgcg

SEQ ID NO: 18 - DNA, 22 bp, synthetic construct
  misc_feature 1..22: note = Synthetic Polynucleotide
  source 1..22: mol_type = other DNA
ccaatcatagaagtcctgcgcg

SEQ ID NO: 19 - DNA, 22 bp, synthetic construct
  misc_feature 1..22: note = Synthetic Polynucleotide
  source 1..22: mol_type = other DNA
accttggccgcatctatgtcgg

SEQ ID NO: 20 - DNA, 21 bp, synthetic construct
  misc_feature 1..21: note = Synthetic Polynucleotide
  source 1..21: mol_type = other DNA
agaacatcatccctgcatcca

SEQ ID NO: 21 - DNA, 21 bp, synthetic construct
  misc_feature 1..21: note = Synthetic Polynucleotide
  source 1..21: mol_type = other DNA
cagatccacgacggacacatt

SEQ ID NO: 22 - DNA, 23 bp, synthetic construct
  misc_feature 1..23: note = Synthetic Polynucleotide
  source 1..23: mol_type = other DNA
cagaacatcatccctgcctctac

SEQ ID NO: 23 - DNA, 23 bp, synthetic construct
  misc_feature 1..23: note = Synthetic Polynucleotide
  source 1..23: mol_type = other DNA
ttgaagtcagaggagaccacctg

SEQ ID NO: 24 - DNA, 2509 bp, Homo sapiens
  source 1..2509: mol_type = other DNA
gaaggcggtggtagcgcctcagtggtgtgggcctgagccctgcccaggtgcccgcagagagcagccgggctgccagcgtttcatgatcaacatgggagactcccacgtggacaccagctccaccgtgtccgaggcggtggccgaagaagtatctcttttcagcatgacggacatgattctgttttcgctcatcgtgggtctcctaacctactggttcctcttcagaaagaaaaaagaagaagtccccgagttcaccaaaattcagacattgacctcctctgtcagagagagcagctttgtggaaaagatgaagaaaacggggaggaacatcatcgtgttctacggctcccagacggggactgcagaggagtttgccaaccgcctgtccaaggacgcccaccgctacgggatgcgaggcatgtcagcggaccctgaggagtatgacctggccgacctgagcagcctgccagagatcgacaacgccctggtggttttctgcatggccacctacggtgagggagaccccaccgacaatgcccaggacttctacgactggctgcaggagacagacgtggatctctctggggtcaagttcgcggtgtttggtcttgggaacaagacctacgagcacttcaatgccatgggcaagtacgtggacaagcggctggagcagctcggcgcccagcgcatctttgagctggggttgggcgacgacgatgggaacttggaggaggacttcatcacctggcgagagcagttctggccggccgtgtgtgaacactttggggtggaagccactggcgaggagtccagcattcgccagtacgagcttgtggtccacaccgacatagatgcggccaaggtgtacatgggggagatgggccggctgaagagctacgagaaccagaagcccccctttgatgccaagaatccgttcctggctgcagtcaccaccaaccggaagctgaaccagggaaccgagcgccacctcatgcacctggaattggacatctcggactccaaaatcaggtatgaatctggggaccacgtggctgtgtacccagccaacgactctgctctcgtcaaccagctgggcaaaatcctgggtgccgacctggacgtcgtcatgtccctgaacaacctggatgaggagtccaacaagaagcacccattcccgtgccctacgtcctaccgcacggccctcacctactacctggacatcaccaacccgccgcgtaccaacgtgctgtacgagctggcgcagtacgcctcggagccctcggagcaggagctgctgcgcaagatggcctcctcctccggcgagggcaaggagctgtacctgagctgggtggtggaggcccggaggcacatcctggccatcctgcaggactgcccgtccctgcggccccccatcgaccacctgtgtgagctgctgccgcgcctgcaggcccgctactactccatcgcctcatcctccaaggtccaccccaactctgtgcacatctgtgcggtggttgtggagtacgagaccaaggctggccgcatcaacaagggcgtggccaccaactggctgcgggccaaggagcctgccggggagaacggcggccgtgcgctggtgcccatgttcgtgcgcaagtcccagttccgcctgcccttcaaggccaccacgcctgtcatcatggtgggccccggcaccggggtggcacccttcataggcttcatccaggagcgggcctggctgcgacagcagggcaaggaggtgggggagacgctgctgtactacggctgccgccgctcggatgaggactacctgtaccgggaggagctggcgcagttccacagggacggtgcgctcacccagctcaacgtggccttctcccgggagcagtcccacaaggtctacgtccagcacctgctaaagcaagaccgagagcacctgtggaagttgatcgaaggcggtgcccacatctacgtctgtggggatgcacggaacatggccagggatgtgcagaacaccttctacgacatcgtggctgagctcggggccatggagcacgcgcaggcggtggactacatcaagaaactgatgaccaagggccgctactccctggacgtgtggagctaggggcctgcctgccccacccaccccacagactccggcctgtaatcagctctcctggctccctcccgtagtctcctgggtgtgtttggcttggccttggcatgggcgcaggcccagtgacaaagactcctctgggcctggggtgcatcctcctcagcccccaggccaggtgaggtccaccggcccctggcagcacagcccagggcctgcatgggggcaccgggctccatgcctctggaggcctctggccctcggtggctgcacagaagggctctttctctctgctgagctgggcccagcccctccacgtgatttccagtgagtgtaaataattttaaataacctctggcccttggaataaagttctgttttctgtaaaaaaaaaaa

SEQ ID NO: 25 - protein, 680 aa, Homo sapiens
MINMGDSHVDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 26 - DNA, 2527 bp, Mus musculus
  source 1..2527: mol_type = other DNA
gggccgtggtagcgcctcagtggtgcgggcttgcgtccggccccagtgcctcagagacctacaggaccgcgcgcggtgtgtgatctggtcggtaccgaggagcgcaggttgtgtcaccaacatgggggactctcacgaagacaccagtgccacagtgcctgaggcagtggctgaagaagtgtctctattcagcacaacggacattgttctgttttctctcatcgtgggggtcctgacctactggttcatctttaaaaagaagaaagaagagataccggagttcagcaagatccagacaacggccccacctgtcaaagagagcagcttcgtggaaaagatgaagaaaacgggaaggaacattattgtattctatggctcccagacgggaaccgcggaggagtttgccaaccggctgtccaaggatgcccaccgctatgggatgcggggcatgtctgcagaccctgaagagtatgacttggccgacctgagcagcctgcctgagatcgacaagtccctggtagtcttctgcatggccacatacggagaaggcgaccccaccgacaacgcgcaggacttctatgattggctgcaggagactgacgtggacctcacgggtgtcaagtttgctgtgtttggtctcgggaacaagacctatgagcacttcaacgccatgggcaagtatgtggaccagcggctggagcagcttggcgcccagcgaatctttgagttgggccttggtgatgacgacgggaacttggaagaggatttcatcacatggagggagcagttctggccagctgtgtgcgagttcttcggggtggaagccactggggaggagtcgagcatccgccagtacgagctcgtggtccacgaagacatggacacagccaaggtgtacacgggtgagatgggccgtctgaagagctacgagaaccagaaaccccccttcgatgccaagaatccattcctggctgctgtcaccacgaaccggaagctgaaccaaggcactgagaggcatctaatgcacctggaattggacatctcagactccaagatcaggtatgaatctggagatcacgtggctgtgtacccagccaacgactccaccctggtcaaccagattggggagatcctgggggctgacctggatgtcatcatgtctctaaacaatctcgatgaggagtcgaataagaagcatccgttcccctgccccaccacctaccgcacggccctcacctactacctggacatcactaacccgccacgaaccaacgtgctctacgagctggcccagtacgcctcagagccctcggagcaggaacacctgcacaagatggcgtcctcctccggcgagggcaaggagctgtacctgagctgggtggtggaggcccggaggcacatcctagccattctccaagactacccgtccctgcggccacccatcgaccacctgtgcgagctcctcccgaggctgcaggcccgctactattccattgcctcgtcgtctaaggtccaccccaactccgtgcacatctgcgccgtggctgtggagtatgaagcgaagtctggacgagtgaacaagggggtggccaccagctggcttcggaccaaggaaccagcaggagagaatggccgccgggccctggtccccatgttcgtccgcaagtcccagttccgcttgcctttcaagcccaccacacctgttatcatggtgggccccggcactggggttgcccctttcatgggcttcatccaggagcgggcttggcttcgagagcaaggcaaggaggtcggagagacgctgctctactacggctgccggcgctcggatgaggactatctgtaccgcgaggagctggcgcgcttccacaaggacggcgccctcacgcagcttaatgtggccttttcccgtgagcaggcccacaaggtctatgttcagcacctgctcaagagggacaaagagcacctgtggaagctgatccacgaaggtggtgcccacatctatgtctgcggggatgctcgaaatatggccaaagatgtgcagaacacattctatgacatcgtggccgagtttgggcccatggagcacacccaggctgtggactatgttaagaagctcatgaccaagggccgctactcgctggatgtatggagctaggagctgccgccccccacccctcgctccctgtaatcacgtccttaacttccttctgccgacctccacctctggtggttcctgccctgcctggacacagggaggcccagggactgactcctggcctgagtgatgccctcctgggcccttaggcagagcctggtccattgtaccaggcagcctagcccagcccagggcacatggcaagagggactggacccacctttgggtgatgggtgccttaggtccccagcagctgtacagaaggggctcttctctccacagagctggggtgcagccccaacatgtgattttgaatgagtgtaaataattttaaataacctggcccttggaataaagttgttttctgta

SEQ ID NO: 27 - protein, 678 aa, Mus musculus
MGDSHEDTSATVPEAVAEEVSLFSTTDIVLFSLIVGVLTYWFIFKKKKEEIPEFSKIQTTAPPVKESSFVEKMKKTGRNIIVFYGSQTGTAEEFANRLSKDAHRYGMRGMSADPEEYDLADLSSLPEIDKSLVVFCMATYGEGDPTDNAQDFYDWLQETDVDLTGVKFAVFGLGNKTYEHFNAMGKYVDQRLEQLGAQRIFELGLGDDDGNLEEDFITWREQFWPAVCEFFGVEATGEESSIRQYELVVHEDMDTAKVYTGEMGRLKSYENQKPPFDAKNPFLAAVTTNRKLNQGTERHLMHLELDISDSKIRYESGDHVAVYPANDSTLVNQIGEILGADLDVIMSLNNLDEESNKKHPFPCPTTYRTALTYYLDITNPPRTNVLYELAQYASEPSEQEHLHKMASSSGEGKELYLSWVVEARRHILAILQDYPSLRPPIDHLCELLPRLQARYYSIASSSKVHPNSVHICAVAVEYEAKSGRVNKGVATSWLRTKEPAGENGRRALVPMFVRKSQFRLPFKPTTPVIMVGPGTGVAPFMGFIQERAWLREQGKEVGETLLYYGCRRSDEDYLYREELARFHKDGALTQLNVAFSREQAHKVYVQHLLKRDKEHLWKLIHEGGAHIYVCGDARNMAKDVQNTFYDIVAEFGPMEHTQAVDYVKKLMTKGRYSLDVWS

SEQ ID NO: 28 - DNA, 1560 bp, Homo sapiens
  source 1..1560: mol_type = other DNA
agaggaaacgtgtgggtggggaggggtagtgggtgagggacccaggttcctgacacagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaaaaaaaaaaaaaaaaaaaaaaaaaaaa

SEQ ID NO: 29 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 30 - DNA, 1663 bp, Mus musculus
  source 1..1663: mol_type = other DNA
aggaaatgtatgggtggggagggcttgtgggagagtggttcagggttctgacacagactacacccagagaaagaagagcaagcaccatgttgaaactattattgtcacctagatccttcttagtccttcagctgctcctgctgagggcagggtggagctccaaggtcctcatgtccagtgcgaatgaagacatcaaagctgatttgatcctgacttctacagcccctgaacacctcagtgctcctactctgccccttccagaggttcagtgctttgtgttcaacatagagtacatgaattgcacttggaatagcagttctgagcctcaggcaaccaacctcacgctgcactataggtacaaggtatctgataataatacattccaggagtgcagtcactatttgttctccaaagagattacttctggctgtcagatacaaaaagaagatatccagctctaccagacatttgttgtccagctccaggacccccagaaaccccagaggcgagctgtacagaagctaaacctacagaatcttgtgatcccacgggctccagaaaatctaacactcagcaatctgagtgaatcccagctagagctgagatggaaaagcagacatattaaagaacgctgtttacaatacttggtgcagtaccggagcaacagagatcgaagctggacggaactaatagtgaatcatgaacctagattctccctgcctagtgtggatgagctgaaacggtacacatttcgggttcggagccgctataacccaatctgtggaagttctcaacagtggagtaaatggagccagcctgtccactgggggagtcatactgtagaggagaatccttccttgtttgcactggaagctgtgcttatccctgttggcaccatggggttgattattaccctgatctttgtgtactgttggttggaacgaatgcctccaattccccccatcaagaatctagaggatctggttactgaataccaagggaacttttcggcctggagtggtgtgtctaaagggctgactgagagtctgcagccagactacagtgaacggttctgccacgtcagcgagattccccccaaaggaggggccctaggagaggggcctggaggttctccttgcagcctgcatagcccttactggcctcccccatgttattctctgaagccggaagcctgaacatcaatcctttgatggaacctcaaagtcctatagtcctaagtgacgctaacctcccctactcaccttggcaatctggatccaatgctcactgccttcccttggggctaagtttcgatttcctgtcccatgtaactgcttttctgttccatatgccctacttgagagtgtcccttgccctctttccctgcacaagccctcccatgcccagcctaacacctttccactttctttgaagagagtcttaccctgtagcccagggtggctgggagctcactatgtaggccaggttggcctccaactcacaggctatcctcccacctctgcctcataagagttggggttactggcatgcaccaccacacccagcatggtccttctcttttataggattctccctccctttttctacctatgattcaactgtttccaaatcaacaagaaataaagtttttaaccaatgatca

SEQ ID NO: 31 - protein, 369 aa, Mus musculus
MLKLLLSPRSFLVLQLLLLRAGWSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEAVLIPVGTMGLIITLIFVYCWLERMPPIPPIKNLEDLVTEYQGNFSAWSGVSKGLTESLQPDYSERFCHVSEIPPKGGALGEGPGGSPCSLHSPYWPPPCYSLKPEA

SEQ ID NO: 32 - DNA, 2457 bp, Homo sapiens
  source 1..2457: mol_type = other DNA
attagatcagtgttcataagaacatctgtaggcacacatacacactctctttacagtcagccttctgcttgccacagtcatagtgggcagtcagtgaatcttccccaagtgctgacaattaatacctggtttagcggcaaagattcagagaggcgtgagcagcccctctggccttcagacaaaaatctacgtaccatcagaaactatgtctctgcagatggtaacagtcagtaataacatagccttaattcagccaggcttctcactgatgaattttgatggacaagttttcttctttggacaaaaaggctggcccaaaagatcctgccccactggagttttccatctggatgtaaagcataaccatgtcaaactgaagcctacaattttctctaaggattcctgctacctccctcctcttcgctacccagccacttgcacattcaaaggcagcttggagtctgaaaagcatcaatacatcatccatggagggaaaacaccaaacaatgaggtttcagataagatttatgtcatgtctattgtttgcaagaacaacaaaaaggttacttttcgctgcacagagaaagacttggtaggagatgttcctgaagccagatatggtcattccattaatgtggtgtacagccgagggaaaagtatgggtgttctctttggaggacgctcatacatgccttctacccacagaaccacagaaaaatggaatagtgtagctgactgcctgccctgtgttttcctggtggattttgaatttgggtgtgctacatcatacattcttccagaacttcaggatgggctatcttttcatgtctctattgccaaaaatgacaccatctatattttaggaggacattcacttgccaataatatccggcctgccaacctgtacagaataagggttgatcttcccctgggtagcccagctgtgaattgcacagtcttgccaggaggaatctctgtctccagtgcaatcctgactcaaactaacaatgatgaatttgttattgttggtggctatcagcttgaaaatcaaaaaagaatgatctgcaacatcatctctttagaggacaacaagatagaaattcgtgagatggagaccccagattggaccccagacattaagcacagcaagatatggtttggaagcaacatgggaaatggaactgtttttcttggcataccaggagacaataaacaagttgtttcagaaggattctatttctatatgttgaaatgtgctgaagatgatactaatgaagagcagacaacattcacaaacagtcaaacatcaacagaagatccaggggattccactccctttgaagactctgaagaattttgtttcagtgcagaagcaaatagttttgatggtgatgatgaatttgacacctataatgaagatgatgaagaagatgagtctgagacaggctactggattacatgctgccctacttgtgatgtggatatcaacacttgggtaccattctattcaactgagctcaacaaacccgccatgatctactgctctcatggggatgggcactgggtccatgctcagtgcatggatctggcagaacgcacactcatccatctgtcagcaggaagcaacaagtattactgcaatgagcatgtggagatagcaagagctctacacactccccaaagagtcctacccttaaaaaagcctccaatgaaatccctccgtaaaaaaggttctggaaaaatcttgactcctgccaagaaatcctttcttagaaggttgtttgattagttttgcaaaagcctttcagattcaggtgtatggaatttttgaatctatttttaaaatcataacattgattttaaaaatacatttttgtttatttaaaatgcctatgttttcttttagttacatgaattaagggccagaaaaaagtgtttataatgcaatgataaataaagtcattctagaccctatacattttgaaaatattttacccaaatactcaatttactaatttattcttcactgaggatttctgatctgattttttattcaacaaaccttaaacacccagaagcagtaataatcatcgaggtatgtttatatttattatataagtcttggtaacaaataacctataaagtgtttatgacaaatttagccaataaagaaattaacacccaaaagaattaaattgattattttgtgcaacataacaattcggcagttggccaaaacttaaaagcaagatctactacatcccacattagtgttctttatataccttcaagcaaccctttggattatgcccatgaacaagttagtttctcatagctttacagatgtagatataaatataaatatatgtatacatatagatagataatgttctccactgacacaaaagaagaaataaataatctacatcaaaaaaaaaaaaaaaaaaaaaaaaaaaa

SEQ ID NO: 33 - protein, 527 aa, Homo sapiens
MSLQMVTVSNNIALIQPGFSLMNFDGQVFFFGQKGWPKRSCPTGVFHLDVKHNHVKLKPTIFSKDSCYLPPLRYPATCTFKGSLESEKHQYIIHGGKTPNNEVSDKIYVMSIVCKNNKKVTFRCTEKDLVGDVPEARYGHSINVVYSRGKSMGVLFGGRSYMPSTHRTTEKWNSVADCLPCVFLVDFEFGCATSYILPELQDGLSFHVSIAKNDTIYILGGHSLANNIRPANLYRIRVDLPLGSPAVNCTVLPGGISVSSAILTQTNNDEFVIVGGYQLENQKRMICNIISLEDNKIEIREMETPDWTPDIKHSKIWFGSNMGNGTVFLGIPGDNKQVVSEGFYFYMLKCAEDDTNEEQTTFTNSQTSTEDPGDSTPFEDSEEFCFSAEANSFDGDDEFDTYNEDDEEDESETGYWITCCPTCDVDINTWVPFYSTELNKPAMIYCSHGDGHWVHAQCMDLAERTLIHLSAGSNKYYCNEHVEIARALHTPQRVLPLKKPPMKSLRKKGSGKILTPAKKSFLRRLFD

SEQ ID NO: 34 - DNA, 3393 bp, Mus musculus
  source 1..3393: mol_type = other DNA
actctaccctgcagccttcagcttggcacaaactaaacagtgactcttccccaagtgccgagtttaattcctggcttggccgaaaggattcagagagggataagcagcccctctggccttcagtgccaaaataagaaagagtatttcacatccacaagcaggaagtacacttcatacctctctaagataaaagacctattcacaatcaaaaatgtccctgcagatggtaacagtgggtcataacatagccttaattcaaccaggcttctcacttatgaattttgatggccaagttttcttctttggccagaaaggctggcctaagagatcctgtcctactggagtctttcattttgatataaaacaaaatcatctcaaactgaagcctgcaatcttctctaaagattcctgctacctcccacctcttcgttatccagctacttgctcatacaaaggcagcatagactctgacaagcatcaatatatcattcacggagggaaaacaccaaacaatgagctttccgataagatttatatcatgtctgtcgcttgcaagaataacaaaaaagttactttccgttgcacagagaaagacttagtaggagatgtccctgaacccagatacggccattccattgacgtggtgtatagtcgagggaaaagcatgggtgttctctttggaggacgttcatacatgccttctacccagagaaccacagaaaaatggaatagtgtagctgactgcctaccccatgttttcttgatagattttgaatttgggtgtgctacatcatatattctcccagaacttcaggatgggctgtcttttcatgtttctattgccagaaacgataccgtttatattttgggaggacactcacttgccagtaatatacgccctgctaacttgtatagaataagagtggaccttcccctgggtaccccagcagtgaattgcacagtcttgccaggaggaatctctgtctccagtgcaatcctcactcaaacaaacaatgatgaatttgttattgtgggtggttatcagctggaaaatcagaaaaggatggtctgcagccttgtctctctaggggacaacacgattgaaatcagtgagatggagactcctgactggacctcagatattaagcatagcaaaatatggtttggaagcaacatgggaaacgggactattttccttggcataccaggagacaataagcaggctatgtcagaagcattctatttctatactttgagatgctctgaagaggatttgagtgaagatcagaaaattgtctccaacagtcagacatcaacagaagatcctggggactccactccctttgaagactcagaggaattttgtttcagtgctgaagcaaccagttttgatggtgacgatgaatttgacacctacaatgaagatgatgaagatgacgagtctgtaaccggctactggataacatgttgccctacttgtgatgttgacatcaatacctgggttccgttctattcaacggagctcaataaacccgccatgatctattgttctcatggggatgggcactgggtacatgcccagtgcatggatttggaagaacgcacactcatccacttgtcagaaggaagcaacaagtattattgcaatgaacatgtacagatagcaagagcattgcaaactcccaaaagaaacccccccttacaaaaacctccaatgaaatccctccacaaaaaaggctctgggaaagtcttgactcctgccaagaaatccttccttagaagactgtttgattaatttagcaaaagcccctcagactcaggtatattgctctctgaatctactttcaatcataaacattattttgatttttgtttactgaaatctctatgttatgttttagttatgtgaattaagtgctgttgtgatttattgttaagtataactattctaatgtgtgttttttaacatcttatccaggaatgtcttaaatgagaaatgttatacagttttccattaaggatatcagtgataaagtatagaactcttacattattttgtaacaatctacatattgaatagtaactaaataccaataaataaactaatgcacaaaaagttaagttcttttgtgtaataagtagcctatagttggtttaaacagttaaaaccaacagctatatcccacactactgctgtttataaattttaaggtggcctctggtttatacttatgagcagaattatatatattggtcaataccatgaagaaaaatttaattctatatcaagccaggcatggtgatggtgatacatgcctgtaatcctggcacttaggaagtggaagaaggaagtttgtgagtttgatgcttgttgaggtatgaccttttgctatgtattgtagtgtatgagccccaagacctgcttgacccagagacaagagagtccacacatagatccaagtaatgctatgtgaccttgccccccggttacttgtgattaggtgaataaagatgtcaacagccaatagctgggcagaagagccaaaagtggggattgagggtaccctggcttgatgtaggaggagaccatgaggaaaggggagaaaaaagtgatggaggaggagaaagatgccatgagctaggagttaagaaagcatggccatgagtgctggccaattggagttaagagcagcccagatgaaacatagtaagtaataactcagggttatcgatagaaaatagattttagtgccgtactctccccagccctagagctgactatggcttactgtaaatataaagtttgtatgtgtcttttatccaggaactaaatggtcaaaggtggagtagaaactctggattgggattaaatttttctacaacaaatgctggcctgggctagattttatctcatatccgaaggctgacagaacacagagcactggtaacattgccacctgccatgcacaaagacctgagtctaatactgtggacattttcttgaagtatctacatgtacttctggagtgaaaacatattccaacaatatgcctttgtttaaatcactcactcactttgggccctcacattatatcctttcaaaatcaatggttcacccctttgaaaatgcttagccatagtccctcatcttccttaaagacagttgtcatctctggaaatagtcacatgtcattcaaggtccaatactgtgcagctctgaagtatggcattaccactttaagtgaaaagtgaaatatgaacatgagctcagacaaaggtttgggactatcactctcaaggaggctctactgctaagtcctgaactgctttcacatgaatacagaaattataacaaaaaatatgtaatcaataaaaagaaaactttcatattcc

SEQ ID NO: 35 - protein, 527 aa, Mus musculus
MSLQMVTVGHNIALIQPGFSLMNFDGQVFFFGQKGWPKRSCPTGVFHFDIKQNHLKLKPAIFSKDSCYLPPLRYPATCSYKGSIDSDKHQYIIHGGKTPNNELSDKIYIMSVACKNNKKVTFRCTEKDLVGDVPEPRYGHSIDVVYSRGKSMGVLFGGRSYMPSTQRTTEKWNSVADCLPHVFLIDFEFGCATSYILPELQDGLSFHVSIARNDTVYILGGHSLASNIRPANLYRIRVDLPLGTPAVNCTVLPGGISVSSAILTQTNNDEFVIVGGYQLENQKRMVCSLVSLGDNTIEISEMETPDWTSDIKHSKIWFGSNMGNGTIFLGIPGDNKQAMSEAFYFYTLRCSEEDLSEDQKIVSNSQTSTEDPGDSTPFEDSEEFCFSAEATSFDGDDEFDTYNEDDEDDESVTGYWITCCPTCDVDINTWVPFYSTELNKPAMIYCSHGDGHWVHAQCMDLEERTLIHLSEGSNKYYCNEHVQIARALQTPKRNPPLQKPPMKSLHKKGSGKVLTPAKKSFLRRLFD

SEQ ID NO: 36 - DNA, 1810 bp, Homo sapiens
  source 1..1810: mol_type = other DNA
gagaccaaaagtcaggtaggagcctccggggtccctgctgtgtcacccggacaggccgtgggggcgggcaggggggcggggccgggcctgaccacagcggccgagttcagtcctgctctccgcacgccaccttaggcccgcagccgtgccgggtgctcttcagcatgtccttcatcccggtggccgaggattccgacttccccatccacaacctgccctacggcgtcttctcgaccagaggcgacccaagaccgaggataggtgtggccattggcgaccagatcctggacctcagcatcatcaagcacctctttactggtcctgtcctctccaaacaccaggatgtcttcaatcagcctacactcaacagcttcatgggcctgggtcaggctgcctggaaggaggcgagagtgttcttgcagaacttgctgtctgtgagccaagccaggctcagagatgacaccgaacttcggaagtgtgcattcatctcccaggcttctgccacgatgcaccttccagccaccataggagactacacagacttctattcctctcggcagcatgctaccaacgtcggaatcatgttcagggacaaggagaatgcgttgatgccaaattggctgcacttaccagtgggctaccatggccgtgcctcctctgtcgtggtgtctggcaccccaatccgaaggcccatgggacagatgaaacctgatgactctaagcctcccgtatatggtgcctgcaagctcttggacatggagctggaaatggctttttttgtaggccctggaaacagattgggagagccgatccccatttccaaggcccatgagcacatttttggaatggtccttatgaacgactggagtgcacgagacattcagaagtgggagtatgtccctctcgggccattccttgggaagagttttgggaccactgtctctccgtgggtggtgcccatggatgctctcatgccctttgctgtgcccaacccgaagcaggaccccaggcccctgccgtatctgtgccatgacgagccctacacatttgacatcaacctctctgttaacctgaaaggagaaggaatgagccaggcggctaccatatgcaagtccaattttaagtacatgtactggacgatgctgcagcagctcactcaccactctgtcaacggctgcaacctgcggccgggggacctcctggcttctgggaccatcagcgggccggagccagaaaacttcggctccatgttggaactgtcgtggaagggaacgaagcccatagacctggggaatggtcagaccaggaagtttctgctggacggggatgaagtcatcataacagggtactgccagggggatggttaccgcatcggctttggccagtgtgctggaaaagtgctgcctgctctcctgccatcatgagattttctctgctcttctggaaacaaagggctcaagcacccctttcaaccctgtgactggggtcctccctcgggctgtaggcctggtccgccattcagtgacaaataaagccattgtgctctgaggcctgcactgccgcagatgcagctgtgtccacttatgatcgtgatttgatccagtgggtcaaggtgtgtaaagcctccctgccagatattcattaatatgttttctcactcttattagtgaggtcaggggtctttgtgggattttcttattagacatcccaggcctcctggtattccatggaatttgaaaagagactggcacctgtagtagtcagggctctccagagaaatagaaccaaggagaaagaaaaaaaaaaaaaa

SEQ ID NO: 37 - protein, 419 aa, Homo sapiens
MSFIPVAEDSDFPIHNLPYGVFSTRGDPRPRIGVAIGDQILDLSIIKHLFTGPVLSKHQDVFNQPTLNSFMGLGQAAWKEARVFLQNLLSVSQARLRDDTELRKCAFISQASATMHLPATIGDYTDFYSSRQHATNVGIMFRDKENALMPNWLHLPVGYHGRASSVVVSGTPIRRPMGQMKPDDSKPPVYGACKLLDMELEMAFFVGPGNRLGEPIPISKAHEHIFGMVLMNDWSARDIQKWEYVPLGPFLGKSFGTTVSPWVVPMDALMPFAVPNPKQDPRPLPYLCHDEPYTFDINLSVNLKGEGMSQAATICKSNFKYMYWTMLQQLTHHSVNGCNLRPGDLLASGTISGPEPENFGSMLELSWKGTKPIDLGNGQTRKFLLDGDEVIITGYCQGDGYRIGFGQCAGKVLPALLPS

SEQ ID NO: 38 - DNA, 1597 bp, Mus musculus
  source 1..1597: mol_type = other DNA
gggtgctaaaagaatcactagggtggggaggcggtcccagtggggcgggtaggggtgtgtgccaggtggtaccgggtattggctggaggaagggcagcccggggttcggggcggtccctgaatctaaaggccctcggctagtctgatccttgccctaagcatagtcccgttagccaaccccctacccgccgtgggctctgctgcccggtgctcgtcagcatgtcctttattccagtggccgaggactccgactttcccatccaaaacctgccctatggtgttttctccactcaaagcaacccaaagccacggattggtgtagccatcggtgaccagatcttggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggagcaggggcagaccaggaccttcctgctggacggcgatgaagtcatcataacaggtcactgccagggggacggctaccgtgttggctttggccagtgtgctgggaaagtgctgcctgccctttcaccagcctgaagctccggaagtcacaagacacacccttgccttatgaggatcatgctaccactgcatcagtcaggaatgaataaagctactttgattgtgggaaatgccacagaaaaaaaaaaaaaaa

SEQ ID NO: 39 - protein, 419 aa, Mus musculus
MSFIPVAEDSDFPIQNLPYGVFSTQSNPKPRIGVAIGDQILDLSVIKHLFTGPALSKHQHVFDETTLNNFMGLGQAAWKEARASLQNLLSASQARLRDDKELRQRAFTSQASATMHLPATIGDYTDFYSSRQHATNVGIMFRGKENALLPNWLHLPVGYHGRASSIVVSGTPIRRPMGQMRPDNSKPPVYGACRLLDMELEMAFFVGPGNRFGEPIPISKAHEHIFGMVLMNDWSARDIQQWEYVPLGPFLGKSFGTTISPWVVPMDALMPFVVPNPKQDPKPLPYLCHSQPYTFDINLSVSLKGEGMSQAATICRSNFKHMYWTMLQQLTHHSVNGCNLRPGDLLASGTISGSDPESFGSMLELSWKGTKAIDVEQGQTRTFLLDGDEVIITGHCQGDGYRVGFGQCAGKVLPALSPA

SEQ ID NO: 40 - DNA, 72 bp, synthetic construct
  misc_feature 1..72: note = Synthetic polynucleotide
  misc_feature 1..72: note = II2-rg WT-guide RNA
  source 1..72: mol_type = other DNA
tccaaggtcctcatgtccagtgcgaatgaagacatcaagcttatccctgttggcacatggggttgattatta

SEQ ID NO: 41 - DNA, 17 bp, synthetic construct
  misc_feature 1..17: note = Synthetic polynucleotide
  misc_feature 1..17: note = Il2-rg genomic deletion
  source 1..17: mol_type = other DNA
tccaaggttgattatta

SEQ ID NO: 42 - DNA, 54 bp, synthetic construct
  misc_feature 1..54: note = Synthetic polynucleotide
  misc_feature 1..54: note = Rag2 WT- guide RNA
  source 1..54: mol_type = other DNA
ccagatacggccattccattgacgtggcctgggttccgttctattcaacggagc

SEQ ID NO: 43 - DNA, 34 bp, synthetic construct
  misc_feature 1..34: note = Synthetic polynucleotide
  misc_feature 1..34: note = Rag2 gene Deletion-1 guide RNA
  source 1..34: mol_type = other DNA
ccagatacggccattccattctattcaacggagc

SEQ ID NO: 44 - DNA, 24 bp, synthetic construct
  misc_feature 1..24: note = Synthetic polynucleotide
  misc_feature 1..24: note = Rag2 genomic Deletion-2 guide RNA
  source 1..24: mol_type = other DNA
ccagatacggccattcaacggagc

SEQ ID NO: 45 - DNA, 20 bp, synthetic construct
  misc_feature 1..20: note = Synthetic polynucleotide
  misc_feature 1..20: note = Rag2 genomic Deletion-3 guide RNA
  source 1..20: mol_type = other DNA
aagttactattcaacggagc

SEQ ID NO: 46 - DNA, 26 bp, synthetic construct
  misc_feature 1..26: note = Synthetic polynucleotide
  misc_feature 1..26: note = Rag2 genomic Deletion-4 guide RNA
  source 1..26: mol_type = other DNA
ccagatacggccatattcaacggagc

SEQ ID NO: 47 - DNA, 52 bp, synthetic construct
  misc_feature 1..52: note = Synthetic polynucleotide
  source 1..52: mol_type = other DNA
gtgcccctgattctgtttcagtgcattatgttgggcattatgttcagaggca

SEQ ID NO: 48 - DNA, 52 bp, synthetic construct; identical to SEQ ID NO: 47
  misc_feature 1..52: note = Synthetic polynucleotide
  misc_feature 1..52: note = Fah genomic Deletion-1 guide RNA
  source 1..52: mol_type = other DNA

SEQ ID NO: 49 - DNA, 40 bp, synthetic construct
  misc_feature 1..40: note = Synthetic polynucleotide
  misc_feature 1..40: note = Fah genomic Deletion-2 guide RNA
  source 1..40: mol_type = other DNA
gtgcccctgattctgtttcagtgcattatgttcagaggca

SEQ ID NO: 50 - protein, 47 aa, synthetic construct
  REGION 1..47: note = Synthetic Polypeptide
  REGION 1..47: note = misc_feature - Il2rg-/- Del-1, Frame shift mutation from aminoacid 28
LCWHYVQRQGECAVAKLAPLTCGIPWPSFLHCGIWNPDSKTHGADET

SEQ ID NO: 51 - protein, 7 aa, synthetic construct
  REGION 1..7: note = Synthetic Polypeptide
  REGION 1..7: note = MISC_FEATURE - Rag2-/- Del-1; Frame shift from aminoacid 143
ILFNGAQ

SEQ ID NO: 52 - protein, 9 aa, synthetic construct
  REGION 1..9: note = Synthetic Polypeptide
  REGION 1..9: note = MISC_FEATURE - Rag2-/- Del-2; Frame shift from aminoacid 141
IQRSSINPP

SEQ ID NO: 53 - protein, 9 aa, synthetic construct
  REGION 1..9: note = Synthetic Polypeptide
  REGION 1..9: note = MISC_FEATURE - Rag2-/- Del-3; Frame shift from aminoacid 122
IQRSSINPP

SEQ ID NO: 54 - protein, 8 aa, synthetic construct
  REGION 1..8: note = Synthetic Polypeptide
  REGION 1..8: note = MISC_FEATURE - Rag2-/- Del-4; Frame shift from aminoacid 142
TKKALGKS

SEQ ID NO: 55 - protein, 47 aa, synthetic construct; identical to SEQ ID NO: 50
  REGION 1..47: note = Synthetic Polypeptide
  REGION 1..47: note = MISC_FEATURE - Fah-/- Del-1; Frame shift from aminoacid 107

SEQ ID NO: 56 - protein, 40 aa, synthetic construct
  REGION 1..40: note = Synthetic Polypeptide
  REGION 1..40: note = MISC_FEATURE - Fah-/- Del-2; Frame shift from aminoacid 107
LCSEARRMRCCQIGSTYLWDTMAELPPLWLEPRFEDPWGR

(19) United States
(12) Patent Application Publication
(10) Pub. No.: US 2025/0268240 A1
(43) Pub. Date: Aug. 28, 2025

(54) HUMAN LIVER CHIMERIC NON-HUMAN ANIMAL WITH DEFICIENT P450 OXIDOREDUCTASE AND METHODS OF USING SAME

(71) Applicant: Baylor College of Medicine, Houston, TX (US)

(72) Inventors: Karl-Dimiter BISSIG, Houston, TX (US); Maria DE LAS MERCEDES BARZI DIEGUEZ, Houston, TX (US); Francis Peter PANKOWICZ, Houston, TX (US)

(21) Appl. No.: 19/208,288

(22) Filed: May 14, 2025

Related U.S. Application Data
(63) Continuation of application No. 18/899,095, filed on Sep. 27, 2024, now abandoned.
Application No. 18/899,095 is a continuation of application No. 18/581,963, filed on Feb. 20, 2024, now abandoned.
Application No. 18/581,963 is a continuation of application No. 18/335,959, filed on Jun. 15, 2023.
Application No. 18/335,959 is a division of application No. 16/309,871, filed on Dec. 13, 2018, now Pat. No. 11,716,974.
(60) Provisional application No. 62/509,942, filed on May 23, 2017.
(60) Provisional application No. 62/355,102, filed on Jun. 27, 2016.

(51) Int. Cl. A01K 67/0271; A01K 67/0276; A61K 49/00

(52) U.S. Cl. CPC A01K 67/0271; A01K 67/0276; A61K 49/0008; A01K 2207/15; A01K 2217/075; A01K 2217/206

(57) ABSTRACT

The present disclosure provides a chimeric non-human animal comprising human hepatocytes, methods for preparing the chimeric non-human animal comprising human hepatocytes and methods of utilizing the chimeric non-human animal comprising human hepatocytes to screening and identifying metabolites for any type of drugs, typically small molecule drugs, which might affect human liver functions and any other bodily function.

### CROSS-REFERENCE TO RELATED APPLICATIONS

This application is a continuation of U.S. patent application Ser. No. 18/899,095, filed Sep. 27, 2024, which is a continuation of U.S. patent application Ser. No. 18/581,963, filed Feb. 20, 2024 which is a continuation of U.S. patent application Ser. No. 18/335,959, filed Jun. 15, 2023 which is a divisional of U.S. application Ser. No. 16/309,871, filed Dec. 13, 2018, now U.S. Pat. No. 11,716,974, which is a National Stage Application, filed under 35 U.S.C. 371, of International Application No. PCT/US2017/039474, filed on Jun. 27, 2017, which claims priority to, and the benefit of, U.S. Provisional Application No. 62/355,102, filed on Jun. 27, 2016, and U.S. Provisional Application No. 62/509,942, filed on May 23, 2017. The entire content of each of these applications is incorporated herein by reference in their entireties.

### INCORPORATION BY REFERENCE OF SEQUENCE LISTING

The Sequence Listing associated with this application is provided electronically in XML file format and is hereby incorporated by reference into the specification. The name of the XML file containing the Sequence Listing is “AVCR-001 C03US SeqList.xml”. The XML file 91.2 kb, was created on May 14, 2025, and is being submitted electronically via the USPTO Patent Center.

### BACKGROUND OF THE INVENTION

Only one out of ten drugs in development gets approved for clinical use. The majority fails during clinical trials due to inefficacy or toxicity in humans. The lack of experimental animal models to accurately predict human xenobiotic metabolism is a significant limitation, which jeopardizes human lives and drives drug development costs. Hence, there is a compelling need to develop better preclinical tools. The present disclosure solves these needs in the art by providing a human liver chimeric non-human animal model and methods of using the human liver chimeric non-human animal model to predict human specific drug metabolism.

### SUMMARY OF THE INVENTION

The present disclosure provides a method for preparing a chimeric non-human animal comprising human hepatocytes, the method comprising: (a) providing a non-human animal comprising a reduction or deletion of NADPH-P450 oxidoreductase (Por) gene resulting in reduced or absent expression of Por protein; and (b) transplanting human hepatocytes into the non-human animal.

The non-human animal can comprise reducing or deleting the Por gene resulting in reduced or absent expression of Por protein. The reduced or deleted Por gene can be a conditional knockdown or knockout of the Por gene. The reduced or deleted Por gene can be the result of a mutation, a transgene, treatment with an exogenous substance or somatic genome engineering, including a CRISPR (Clustered regularly interspaced short palindromic repeats) system. The somatic genome engineering comprises Guide RNA (gRNA) and Caspase 9 (Cas9).

The non-human animal can comprise a floxed allele of the Por gene, and wherein the non-human animal is provided with a Cre recombinase sufficient to produce a conditional knockout of the Por gene. The non-human animal comprising the floxed allele of the Por gene can be provided with at least a first dose of a virus that encodes Cre recombinase. The non-human animal can be provided with at least a second dose of a virus that encodes Cre recombinase. The non-human animal can comprise the floxed allele of the Por gene is crossed with a transgenic non-human animal strain expressing Cre recombinase.

In a one aspect, the method of the present disclosure comprises (a) providing a non-human animal comprising a floxed allele of the Por gene with a first does of a virus that encodes Cre recombinase; (b) transplanting human hepatocytes into the non-human animal; and (c) providing the non-human animal with a second dose of a virus that encodes Cre recombinase. Steps (a) and (b) can occur sequentially or simultaneously.

The non-human animal can further comprise a reduction or deletion of at least one additional gene encoding an enzyme involved in drug metabolism. The at least one additional enzyme can be a phase II drug enzyme. In one aspect, the non-human animal can further comprise a reduction or deletion of UDP-glucose 6-dehydrogenase (UGDH) gene, a reduction or deletion of Glutathione synthetase (GSS) gene, or a combination thereof.

The reduction or deletion the UGDH gene can result in reduced or absent expression of UGDH protein. The reduction or deletion the GSS gene can result in reduced or absent expression of GSS protein. The reduced or deleted UGDH gene can be a conditional knockdown or knockout of the UGDH gene. The reduced or deleted GSS gene can be a conditional knockdown or knockout of the GSS gene. The reduced or deleted UGDH or GSS gene can be the result of a mutation, a transgene, treatment with an exogenous substance or somatic genome engineering, including a CRISPR (Clustered regularly interspaced short palindromic repeats) system. The somatic genome engineering comprises Guide RNA (gRNA) and Caspase 9 (Cas9).

The non-human animal can be selected from the group consisting of primate, bird, mouse, rat, fowl, dog, cat, cow, horse, goat, camel, sheep and pig. In a preferred aspect, the non-human animal is a mouse.

The non-human animal comprising a reducing or deleting the Por gene can be selected from the group consisting of (i) the FRG (Fah^{-/-}/Rag2^{-/-}Il2rg^{-/-}) non-human animal, (ii) a transgenic urokinase type plasminogen activator (uPA) non-human animal, which overexpress uPA under an inducible promoter, preferably a liver-restricted albumin promoter, (iii) the thymidine kinase-NOD/Shi-scid/IL-2Rγ^{null} (TK-NOG) non-human animal, which is a immunodeficient NOG non-human animal with transgenic expression of thymidine kinase under control of liver-restricted promoter, (iv) a non-human animal expressing an inducible Caspase 8 in the liver, and (v) a non-human animal expressing an inducible Caspase 9 in the liver.

The present disclosure also provides a chimeric non-human animal, offspring thereof, or a portion thereof, which has a chimeric liver comprising human hepatocytes, prepared by any method disclosed herein.

The chimeric non-human animal can be immunodeficient. The chimeric non-human animal substantially lacks autogenous hepatocytes. Human hepatocytes can account for any percentage of human chimerism greater than about 1%, for example at least 5%, at least 10%, at least 20%, at least 30%, at least 40%, at least 50%, at least 60%, at least 70%, at least 80%, at least 90%, at least 95% or at least 99% of all hepatocytes in the chimeric liver of the chimeric non-human animal. A “non-human animal” can be amphibian, reptile, avian, or a non-human mammal. The non-human animal can be e.g., any non-human mammal, e.g., primate, bird, mouse, rat, fowl, dog, cat, cow, horse, goat, camel, sheep or pig. In a preferred aspect, the non-human animal is a mouse.

In one aspect, the present disclosure provides a method for preparing a chimeric non-human animal comprising human hepatocytes, the method comprising steps of: (a) providing a non-human animal that allows its liver to be repopulated with human hepatocytes and comprising a non-functional NADPH-P450 oxidoreductase generated either by genome engineering or knockdown with exogenous agents such as genome engineering tools like CRISPR/Cas9 or floxed allele of the NADPH-P450 oxidoreductase (Por) gene with a first dose of a virus that encodes Cre recombinase or a Cre transgenic animal, thereby producing a conditional knockout of the Por gene; (b) transplanting human hepatocytes into the non-human animal; and (c) providing the non-human animal with a second dose of the virus that encodes Cre recombinase. The chimeric non-human animal can substantially lack autogenous or endogenous hepatocytes and instead comprising human hepatocytes. Steps (a) and (b) can occur sequentially or simultaneously. Any non-human animal comprising mutations and/or transgenes that allow its liver to be repopulated with human hepatocytes may be used in combination with the floxed or deleted allele of the NADPH-P450 oxidoreductase (Por) gene or functional inactivation of the Por protein. In aspects, the non-human animal comprising mutations and/or transgenes that allow its liver to be repopulated with human hepatocytes is (i) the FRG (Fah^{-/-}/Rag2^{-/-}/Il2rg^{-/-}) non-human animal, (ii) a transgenic uPA non-human animal, which overexpress urokinase type plasminogen activator (uPA) in the liver under an inducible promoter and/or preferably a liver-restricted albumin promoter, (iii) the TK-NOG non-human animal, which is a immunodeficient NOG non-human animal with transgenic expression of thymidine kinase under control of liver-restricted albumin promoter, (iv) a non-human animal expressing an inducible Caspase 8 in the liver, or (v) a non-human animal expressing an inducible Caspase 9 in the liver (vi) a non-human animal expressing human heparin-binding epidermal growth factor-like receptor (HB-EGF)-like receptors under the control of a liver cell-specific albumin promoter (alb-TRECK). A “non-human animal” can be amphibian, reptile, avian, or a non-human mammal.

In one aspect, the present disclosure provides a method for preparing a chimeric mouse substantially lacking murine hepatocytes and instead comprising human hepatocytes, comprising steps of: (a) providing a mouse that allows its liver to be repopulated with human hepatocytes and comprising a non-functional NADPH-P450 oxidoreductase generated either by genome engineering by CRISPR/Cas9 mediated deletion or knockdown with exogenous agents or floxed allele of the NADPH-P450 oxidoreductase (Por) gene with a first dose of a virus that encodes

Cre recombinase or a Cre transgenic mouse, thereby producing a conditional knockout of the Por gene; (b) transplanting human hepatocytes into the mouse; and (c) providing the mouse with a second dose of the virus that encodes Cre recombinase. Steps (a) and (b) can occur sequentially or simultaneously. Any mouse that allow its liver to be repopulated with human hepatocytes may be used in combination with the floxed allele of the NADPH-P450 oxidoreductase (Por) gene or somatic gene deletion or reduction or inactivation of the Por gene, respectively protein. In aspects, the mouse that allow its liver to be repopulated with human hepatocytes is (i) the FRG (Fah^{-/-}/Rag2^{-/-}/Il2rg^{-/-}) mouse, (ii) a transgenic uPA mouse, which overexpress urokinase type plasminogen activator (uPA) under an inducible promoter, preferably a liver-restricted albumin promoter, (iii) the TK-NOG mouse, which is a super immunodeficient NOG mouse with transgenic expression of thymidine kinase under control of liver-restricted albumin promoter, (iv) a mouse expressing an inducible Caspase 8 in the liver, (v) a mouse expressing an inducible Caspase 9 in the liver or (vi) a mouse expressing human heparin-binding epidermal growth factor-like receptor (HB-EGF)-like receptors under the control of a liver cell-specific albumin promoter (alb-TRECK).

The present disclosure also provides a method for screening and identifying metabolites for any type of drugs, typically small molecule drugs, that might affect human liver functions but also any other function of the body, comprising: (a) administering a test substance to the chimeric non-human animal of the present disclosure; (b) measuring one or more values in the chimeric non-human animal to which the test substance is administered in (a); and (c) selecting a test substance that causes an increase or an decrease in one or more values measured in (b), compared with the one or more values measured in a chimeric non-human animal to which no test substance is administered or a chimeric non-human animal without deletion of the Por gene or a non-human animals without human chimerism. Preferably, the one or more values are selected from but not limited to the group consisting of a metabolite of the test substance, human albumin concentration, body weight curve, liver-weight-to-body-weight ratio, total albumin level, total protein level, Alanine Aminotransferase (ALT) level, Aspartate Aminotransferase (AST) level, and total bilirubin level, creatinine, Blood Urea Nitrogen (BUN), troponine, blood count, TSH and histological assessment for pathologies in the human and non-human organs. A “non-human animal” can be amphibian, reptile, avian, or a non-human mammal. The non-human animal can be e.g., any non-human mammal, e.g., primate, bird, mouse, rat, fowl, dog, cat, cow, horse, goat, camel, sheep or pig. Preferably, the non-human animal is a mouse.

The present disclosure further provides a method for screening for a substance that affects human liver functions, comprising: (a) administering a test substance to the chimeric mouse of the present disclosure; (b) measuring one or more values in the chimeric mouse to which the test substance is administered in (a); and (c) selecting a test substance that causes an increase or an decrease in one or more values measured in (b), compared with the one or more values measured in a chimeric mouse to which no test substance is administered. Preferably, the one or more values is selected from the group consisting of a metabolite of the test substance, human albumin concentration, body weight curve, liver-weight-to-body-weight ratio, total albumin level, total protein level, ALT level, AST level, and total bilirubin level, histological assessment for pathologies in the human and non-human organs.

The present disclosure also provides a method for evaluating the toxicity of a test substance against human hepatocytes, comprising: (a) administering a test substance to the chimeric non-human animal of the present disclosure; (b) measuring one or more indicators in the chimeric non-human animal to which the test substance is administered in (a); and (c) evaluating the effect of the test substance on human hepatocytes using, one or more indicators measured in (b), compared with the one or more indicators measured in a chimeric non-human animal to which no test substance is administered. Preferably, the one or more indicators is selected from the group consisting of an increase or a decrease in any one or more of a metabolite of the test substance, human albumin concentration, body weight curve, liver-weight-to-body-weight ratio, total albumin level, total protein level, ALT level, AST level, and total bilirubin level, histological assessment for toxicity in the human and non-human organs. A “non-human animal” can be amphibian, reptile, avian, or a non-human mammal. The non-human animal can be e.g., any non-human mammal, e.g., primate, bird, mouse, rat, fowl, dog, cat, cow, horse, goat, camel, sheep or pig. Preferably, the non-human animal is a mouse.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

### BRIEF DESCRIPTION OF THE DRAWINGS

The patent or application file contains at least one drawing executed in color. Copies of this patent or patent application publication with color drawings will be provided by the Office upon request and payment of the necessary fee.

The above and further features will be more clearly appreciated from the following detailed description when taken in conjunction with the accompanying drawings.

FIG. 1A-D shows the generation of the PIRF strain and deletion of the murine P450 (Por) oxidoreductase. FIG. 1A is a schematic representation of deleted and transgenic loci in the PIRF strain. FIG. 1B is a graph showing qPCR of Por mRNA upon intravenous injection of adenovirus expressing the CRE recombinase (Adeno-Cre). FIG. 1C is a series of immunostaining photographs for Por demonstrating a gradient across the hepatic acinus with higher pericentral (cv) and lower periportal (pv) expression. Upon injection with Adeno-CrePor is barely detectable. FIG. 1D is a photograph of a Western blot showing the almost complete disappearance of Por protein.

FIG. 2A-E shows the humanization of the PIRF strain and gene expression profiling upon deletion of murine P450 oxidoreductase (Por). FIG. 2A is a series of immunostaining photographs showing humanized PIRF and FRG mice for murine Por (mPor) and human nuclei (hNuc) after injection of Adeno-Cre (2.2×10¹⁰ pfu/mouse) once (1×) or twice (2×). Counterstaining in merged picture using DAPI. FIG. 2B is a schematic showing the experimental outline for murine and human transcriptomics from chimeric livers with or without Por deletion. FIG. 2C is a graph showing the murine cytochrome mRNA originating from livers of the PIRF model. FIG. 2D is a graph showing the human cytochrome mRNA originating from livers of the PIRF model. FIG. 2E is a graph showing the comparison gene expression of the main drug metabolizing human cytochromes in humanized, Por-deleted PIRF (Hu-PIRF 2x) mice with the original, isogenic human hepatocytes. Gene expression has been normalized to three murine respectively human housekeeping genes (PSMB2, PSMB4 and RAB7A resp. Rab7²⁵). PIRF; Por^{c/c}/Il2rg^{-/-}/Rag2^{-/-}, Fah^{-/-}FRG; Fah^{-/-}/Rag2^{-/-}/Il2rg^{-/-}.

FIG. 3A-E shows xenobiotic metabolism in humanized PIRF mice. FIG. 3A is a graph showing the selection of abundant and decreased gefitinib metabolites upon murine P450 oxidoreductase (Por) deletion in non-humanized PIRF mice using mass spectrometry in the murine feces within 24 hours after intravenous injection of gefitinib. FIG. 3B is a schematic showing gefitinib metabolites and know modifications. FIG. 3C is a graph showing that murine Por-deleted, human liver chimeric PIRF (Hu-PIRF 2x) mice and control groups show the most abundant human metabolite, M4. FIG. 3D is a graph showing that murine Por-deleted, human liver chimeric PIRF (Hu-PIRF 2x) mice and control groups show the human specific metabolite M28. PIRF; Por^{c/c}/Il2rg^{-/-}/Rag2^{-/-}, Fah^{-/-}, FRG; Fah^{-/-}/Rag2^{-/-}/Il2rg/^{-/-}. *p<0.05 using non-parametric Mann-Whitney test. FIG. 3E is a graph is a graph showing a mass spectrometry analysis of PIRF liver homogenates 30 min after injection with atazanavir, a retroviral therapeutic. A major human metabolite (M15) is shown. The overall abundance of metabolites from atazanavir or gefitinib was set as 100% in each sample. The data are expressed as mean. PIRF; Por^{c/c}/Il2rg^{-/-}/Rag2^{-/-}/Fah^{-/-}, FRG; Fah^{-/-}/Rag2^{-/-}/Il2rg^{-/-}*p<0.05 using non-parametric Mann-Whitney test.

FIG. 4A-B shows a schematic of an exemplary probe of the present disclosure. FIG. 4A is a schematic showing the design of the targeting vector and modified Por locus. FIG. 4B is a photograph of Southern blotting with three probes demonstrating proper targeting of ESC.

FIG. 5 shows beta galactosidase (lacZ) expression from the POR-lacZ allele. X-gal staining of heterozygous mouse embryo and liver demonstrate expression of the galactosidase from the Por-lacZ allele.

FIG. 6A shows a scheme of Il2 rg, Rag2 and Fah genes showing the gRNA location (in color) and the primers used to genotype the mice. FIG. 6B shows an electrophoresis gel of the PCR products obtained from genotyping. Lane 1: PCR bands using external (Fw and Rv) primers. For Rag2 and Fah heterozygotes, a wild type (arrow) and deleted (arrowhead) allele can be detected. Il2 rg is an X-linked gene and no founder heterozygote females were generated. Homozygotes mice for Il2 rg, Rag2 and Fah show a single deleted allele's band of 460 bp, 530 bp and 200 bp, respectively. Lane 2: PCR bands using one of the external (Fw or Rev) and the internal (Int) primer located between the two gRNA sites. Only heterozygotes have a clear PCR band formed from the wild type allele. Fah and Rag2 homozygotes show multiple unspecific bands while Il2 rg homozygotes do not produce any band.

FIGS. 7A and 7B show the spectrum of genomic deletions in the Il2-rg, Rag2 and Fah gene. FIG. 7A is a schematic showing CRISPR/Cas9 injected zygotes of conditional Por−/− mice sequenced to determine of deletion of DNA. FIG. 7B is a schematic showing CRISPR/Cas9 injected zygotes of conditional Por−/−mice sequenced to determine of deletion of amino acids.

FIG. 8 shows lipid phenotype in the liver of PIRF mice upon deletion of the Por gene using an adenovirus expressing the CRE recombinase. Two weeks after injection of the adenovirus hepatocytes start to accumulated lipids. Oil-red-O stained livers have previously been validated for efficient deletion (upper panel) respectively expression (lower panel) of the Por gene (not shown).

FIG. 9 shows clonal expansion of Por expressing hepatocytes in non-humanized PIRF mice. Mice were injected intravenously with adenovirus expressing CRE recombinase deleting the Por gene (POR^{fl/fl})

FIG. 10 shows the experimental setup for drug studies in humanized and non-humanized control mice. Humanized (Hu) and not-humanized PIRF and FRG ice were injected once (1×, before transplantation of human hepatocytes) or twice (2×, before and after reaching high human chimerism) before doing drug studies. Injected adenovirus expresses the CRE-recombinase, which leads to deletion of the Por gene in the PIRF, but not in the FRG strain (control). Orange; murine drug metabolism, white; inhibited murine drug metabolism, blue; human drug metabolism.

FIGS. 11A and 11B show that the conditional KO of POR can also be generated using a transgenic animal, which carries an expression cassette (albumin promoter) of the CRE recombinase. FIG. 11A shows that POR can be detected in immunofluorescence with the control group where only one allele carries a floxed POR sequence the other one the wild-type POR (POR^{c/+}). In FIG. 11B, both POR alleles are floxed (POR^{c/c}), leading to an almost complete deletion of the POR gene and a barely detectable POR protein. POR (green) and nuclei (blue, DAPI).

FIG. 12 depicts the expression of P450 oxidoreductase in humanized PIRF mice. FIG. 12A is a bar graph showing qPCR of human and murine specific Por normalized to human and murine Gapdh, respectively. FIG. 12B is a western blot image showing murine Por and B-actin of liver samples from the same humanized mice.

FIG. 13 depicts Gefitinib metabolites upon murine P450 oxidoreductase (Por) deletion. FIG. 13A is a bar graph showing deletion by adenoviral delivery of CRE. FIG. 13B is a bar graph showing by crossing of Por^{c/c} with Alb-Cre mice, generating an Alb-Cre/Por^{c/c} strain.

FIG. 14 depicts the deletion of murine P450 oxidoreductase by crossing Por^{c/c} with an Alb-Cre transgenic mouse. FIG. 14A is a confocal immunostaining image showing complete Por deletion. FIG. 14b is a western blot image showing Por protein liver samples from a control Porc/c mouse and three different Alb-Cre/Porc/c mice. FIG. 14C is a bar graph showing qPCR of murine Por mRNA levels.

FIG. 15 is a bar graph showing Gefitinib metabolite M28 in feces.

FIG. 16 is a pair of bars graphs showing Gefitinib metabolite M28 in the (A) serum and (B) urine of hu-PIRF-2x mice and control groups.

FIG. 17 is a Southern blot image of targeted ESCs. FIG. 17A shows the use of a 5′probe, FIG. 17B shows the use of a 3′ probe and FIG. 17C shows the use of a neomycin probe.

FIG. 18 is a Western blot image showing murine Por and Gadph from PIRF mice injected with Adeno-CRE.

FIG. 19A-B is a Western blot image showing murine Por and Gadph from Humanized PIRF mice (a) and Por^{c/c} and (b) Por^{c/c}/Alb-CRE mice.

FIG. 20 is an H&E stain of liver lobe four weeks after transduction with adenovirus showing macro-and microvesicular steatosis. Left picture is a higher magnification of boxed area on right FIG. 21A-B shows gene therapy vector design for liver-specific deletion by genome engineering of drug metabolizing enzymes in humanized mice. A. Two vector design: S. pyogenes Cas9 under the control of CMV promoter in an adenoviral vector (Ad), and Adeno-Associated Virus (AAV) expressing a sgRNA targeting a drug metabolizing enzyme and co-expression of GFP on the same construct. FIG. 21B. Single vector design: S. aureus Cas9 and sgRNA can be delivered on the same AAV vector (4.85 kb). HA, HA-epitope.

FIG. 22 shows simultaneous deletion of murine P450 oxidoreductase (Por) and other murine enzymes involved in drug metabolism in humanized mice by somatic genome engineering. Humanized FRG mice (human albumin in murine serum >2 mg/ml) have been injected with Adeno-Associated Virus (AAV, serotype 8) expressing sgRNA targeting an early exon of murine Por,UDP-glucose 6-dehydrogenase (Ugdh) or the glutathione synthetase (Gss) gene (see gene therapy vector design, FIG. 21). AAVs have been injected (2×10¹¹ GC/AAV/mouse) 1 week before injection of Adenovirus expressing Cas9 (7×10⁹ pfu/Ad/mouse). Control mice have been injected with adenoviral vector only (lower row). Shown are serial sections of a representative humanized area with immunostaining for human specific Pre-ALB (transthyretin), Por, Ugdh, Gss, hALB and GFP, the latter being expressed from the AAV gene therapy vector (see gene therapy vector design, FIG. 21). Bar represents 50 μm. ALB, Albumin.

FIG. 23 shows genomic deletion of por by CRISPR/Cas9: Wild type and humanized FRG mice (human albumin in murine serum >2 mg/ml) are injected with two Adeno-Associated Virus (AAV, serotype 8) expressing sgRNA targeting consecutive early exons of murine Por as well as S. aureus Cas9 (see gene therapy vector design, FIG. 21). AAVs are injected (2×10¹¹ GC/AAV/mouse). Control mice are injected with adenoviral vector expressing Cas9 only (7×10⁹ pfu/Ad/mouse). Shown are PCR amplifications of the region spanning the two nearby targeting sites. Upon CRISPR/Cas9 mediated cutting of DNA on both target sites results in a deletion of the por gene leading to a smaller PCR amplicon.

FIG. 24 shows humanized PIRF mouse with transgenic Alb-CRE and deletion of other murine enzymes involved in drug metabolism. Por was deleted by expression of CRE, but instead of adenoviral CRE, this PIRF mouse carries an Alb-CRE sequence within the murine genome. After humanization, mice were injected with Adeno-Associated Virus (AAV, serotype 8) expressing sgRNA targeting an early exon UDP-glucose 6-dehydrogenase (Ugdh) or the glutathione synthetase (Gss) gene (see gene therapy vector design, FIG. 21). AAVs are injected (2×10¹¹ GE/AAV/mouse) 1 week before injection of Adenovirus expressing Cas9 (7×10⁹ GE/Ad/mouse). Shown are serial sections of a representative humanized area with immunostaining for human specific Pre-ALB (transthyretin), Por, Ugdh or Gss. Bar represents 50 μm. ALB, Albumin.

FIG. 25A-D shows troglitazone Phase II metabolites detected in liver of humanized and non-humanized FRG mice with and without Por and Ugdh deletion. Percentage of liver glucuronidated and sulfated metabolites detected in humanized livers 2 hours after i.p injection of troglitazone (600 mg/kg). FIG. 25A. Humanized FRG mice. FIG. 25B. Humanized FRG mice after murine Por and Ugdh deletion. FIG. 25C. Non-humanized FRG mice. FIG. 25D. Non-humanized FRG mice with Por and Ugdh genes deleted. Sulfate metabolites of troglitazone in red and glucuronide conjugates in blue.

### DETAILED DESCRIPTION OF THE INVENTION

Human liver chimeric mice have been recently introduced to predict human xenobiotic metabolism and toxicity. Despite their potential, the remaining murine liver, containing an expanded set of P450 cytochromes, makes it difficult to accurately predict human drug metabolism. Therefore, the present disclosure provides a conditional knock-out mouse of the NADPH-P450 oxidoreductase (Por) gene, which is the only electron donor for all murine cytochromes and if deleted, embryonically lethal¹, thereby allowing a functional inactivation of all murine cytochromes.

Any mouse comprising mutations and/or transgenes that allow its liver to be repopulated with human hepatocytes may be used in combination with the conditional knock-out allele or other genomic deletion of the NADPH-P450 oxidoreductase (Por) gene. In embodiments, the mouse comprising mutations and/or transgenes that allow its liver to be repopulated with human hepatocytes is (i) the FRG (Fah^{-/-}/Rag2^{-/-}/Il2rg^{-/-}) mouse, (ii) a transgenic uPA mouse, which overexpress urokinase type plasminogen activator (uPA) under an inducible promoter, preferably a liver-restricted albumin promoter, (iii) the TK-NOG mouse, which is a immunodeficient NOG mouse with transgenic expression of thymidine kinase under control of liver-restricted albumin promoter, (iv) a mouse expressing an inducible Caspase 8 in the liver, (v) a mouse expressing an inducible Caspase 9 in the liver or (vi) a mouse expressing human heparin-binding epidermal growth factor-like receptor (HB-EGF)-like receptors under the control of a liver cell-specific albumin promoter (alb-TRECK). Using such mice and an adenoviral or transgenic strategy expressing CRE, an almost complete deletion of the murine Por gene can be generated leading to an exclusive human cytochrome metabolism.

In the uPA-SCID mouse (Rhim et al 1994; Tateno et al. 2004), the genetic cause of mouse hepatocyte ablation is uroplasminogen activator (uPA); the mouse is in the SCID immune deficient background or Rag2 (or Rag1)−/− and/or Il2rg−/− all leading to the ability to transplant and engraft human hepatocytes.

In the FRG mouse (Azuma et al 2007⁷, Bissig et al 2007⁵), the genetic cause of mouse hepatocyte ablation is fumarylacetoacetate hydrolase deficiency and mouse hepatocyte ablation is controlled by ±NTBC and/or ±low tyrosine diet; the mouse is in the Il2rg−/− and Rag2−/− background. The FRG mouse combines immune-deficiency-mediating mutations, in the recombination activating gene 2 (Rag2) and the gamma chain of the interleukin 2 receptor (Il2rg), with a functional knockout of the fumarylacetoacetate hydrolase (Fah) gene (Azuma et al 2007⁷, Bissig et al 2007⁵). The latter gene codes for an enzyme in the tyrosine catabolic pathway and its mutation leads to an intracellular accumulation of a toxic inter-mediate in hepatocytes. Unlike the uPA/SCID model, the onset and severity of hepatocellular injury in FRG mice is controllable through the administration and withdrawal of the protective drug 2-(2-nitro-4-trifluoromethylbenzoyl)-1,3-cyclohexanedione (NTBC), which blocks an upstream enzyme in the tyrosine path-way and thereby prevents accumulation of the toxic intermediate.

In the TK-NOG mouse (Hasegawa et al 2011), the genetic cause of mouse hepatocyte ablation is the herpes simplex virus thymidine kinase and mouse hepatocyte ablation is controlled by ±ganciclovir; the mouse is in the Il2rg−/− and SCID background. Mouse hepatocyte ablation in this TK-NOG model was achieved through the liver-specific expression of the herpes simplex virus 1 thymidine kinase (HSVtk) in severely immunodeficient NOG mice and administration of ganciclovir (GCV), utilizing the fact that HSVtk converts the otherwise nontoxic GCV into a toxic intermediate.

In the AFC8 mouse (Washburn et al 2011), the genetic cause of mouse hepatocyte ablation is a FK508-capsae 8 fusion and mouse hepatocyte ablation is controlled by ±AP20187; the mouse is in the Il2rg−/− and Rag2−/− background.

In the Alb-TRECK/SCID mouse (Zhang et al 2015), the genetic cause of mouse hepatocyte ablation is the human heparin-binding EGF-like receptor and mouse hepatocyte ablation is controlled by +Diphtheria toxin; the mouse is in the SCID immune deficient background.

Sheer and Wilson, 2015 compares major features of various different liver humanized models and process of liver reconstitution in the most frequently used models to date. This reference is incorporated by reference in its entirety.

The present disclosure also provides methods of utilizing the humanized, murine Por deficient mice to predict human drug metabolism. In an embodiment, the FRG mouse and the conditional Por−/−mouse was combined to generate the PIRF (Por−/−Il2rg−/−/Rag2−/−/Fah−/−) strain, which allows repopulation with human hepatocytes. Homozygous PIRF mice are fertile and can be repopulated with human hepatocytes generating high human chimerism (>80% human).

Human p450 cytochrome clusters contain 57 putatively functional genes and 58 pseudogenes, while the mouse cytochrome clusters are greatly expanded accounting for 102 putatively functional genes and 88 pseudogenes². This makes accurate prediction of human drug metabolism in the mouse challenging. In addition hepatotoxicity together with hypersensitivity/cutaneous reactions have the poorest correlation with animal studies yet are the most common reasons for toxicity related termination of drugs in clinical development³.

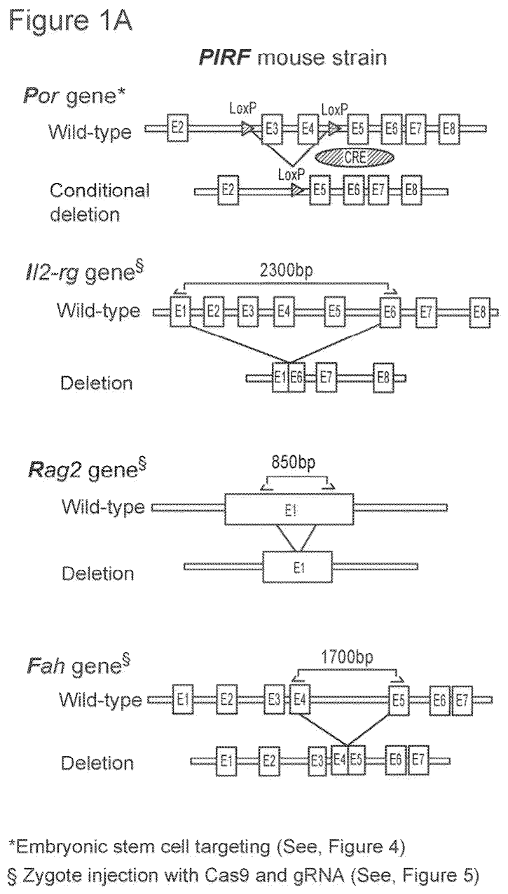

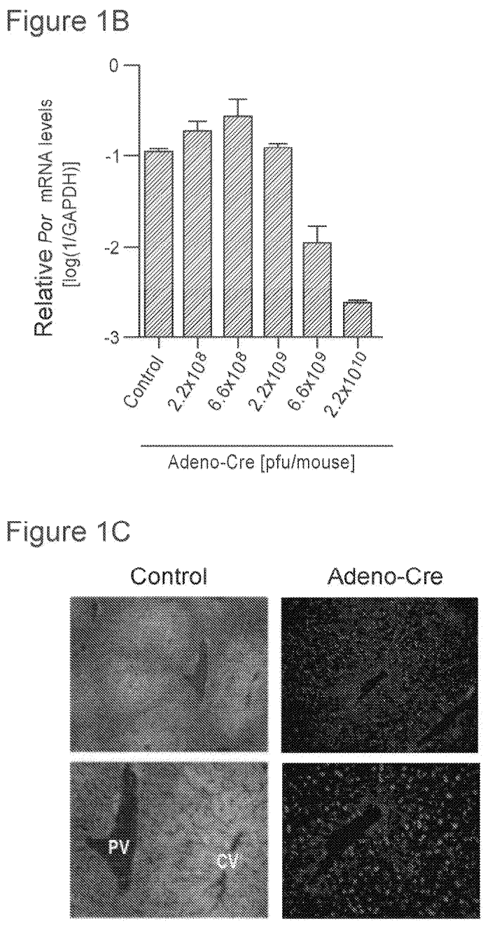

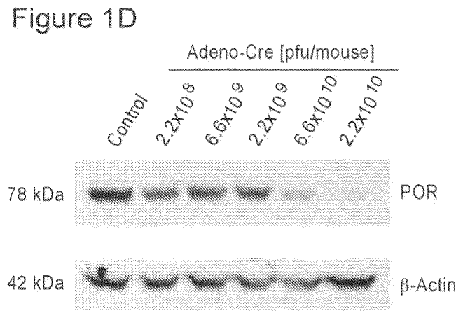

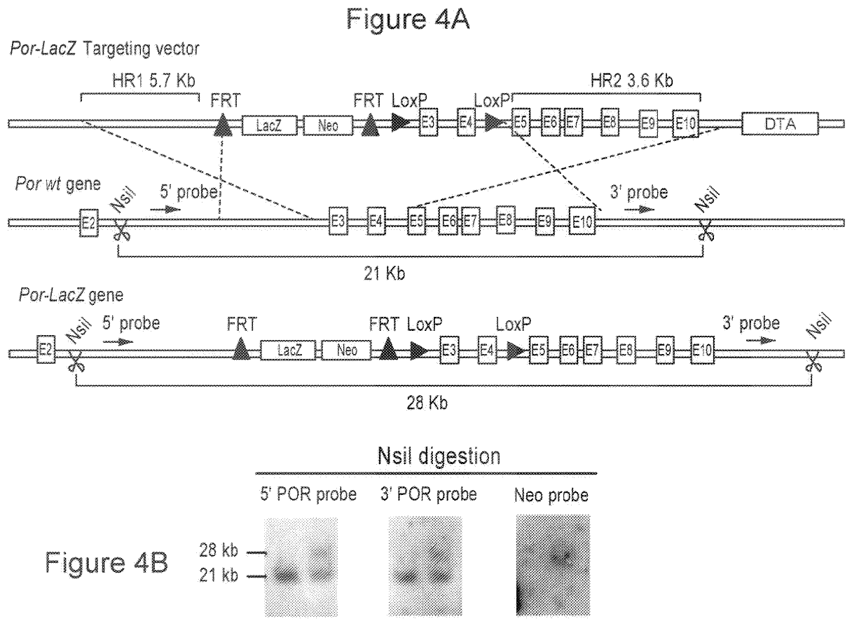

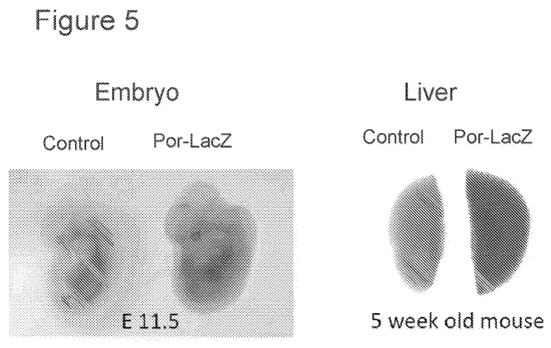

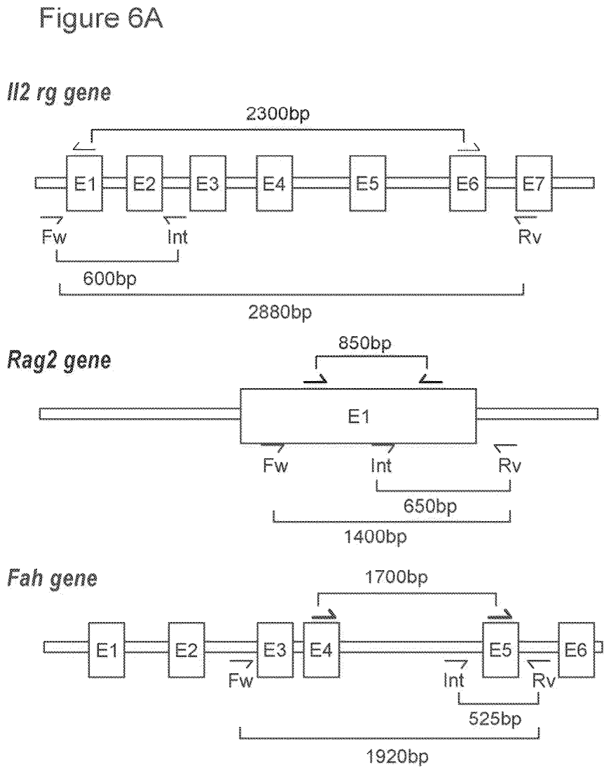

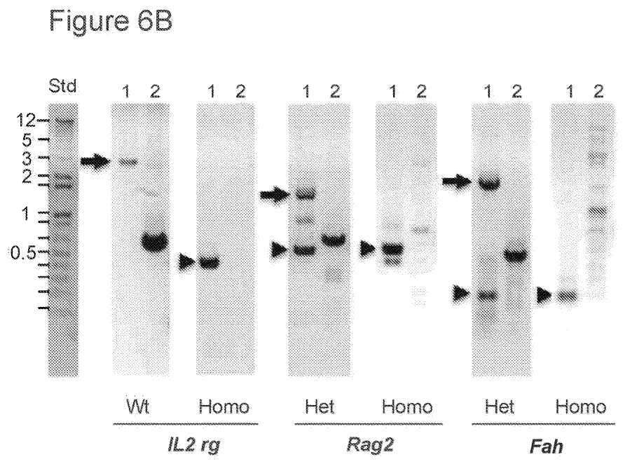

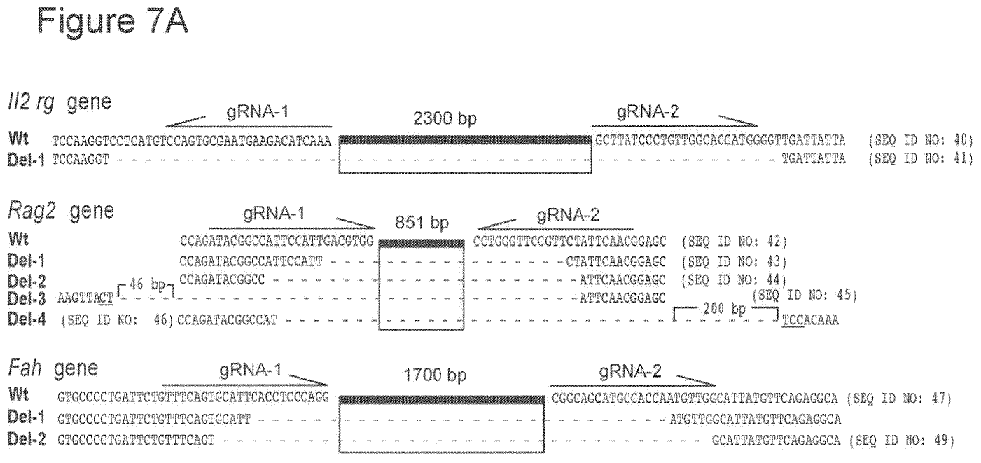

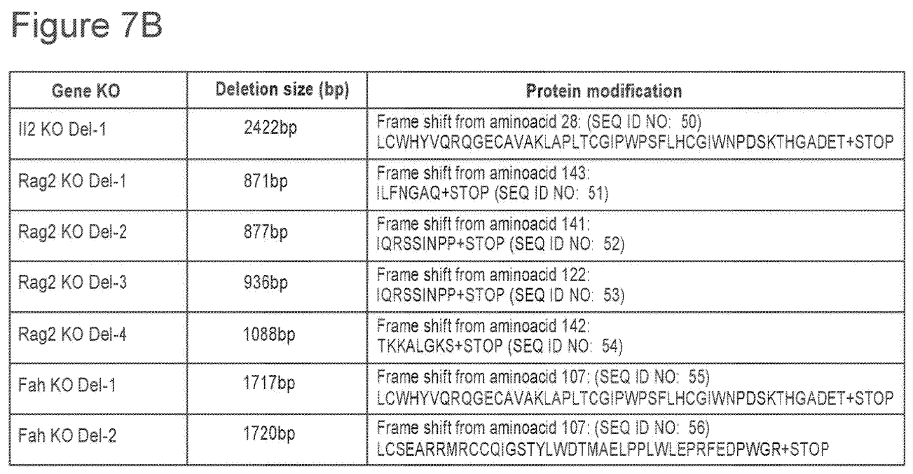

Since the liver is the main organ for drug metabolism, human liver chimeric mice are increasingly used for xenobiotic studies⁴⁻⁶. The shortcoming of humanized mice is the remaining murine liver tissue. It has been previously shown that even in mice that can achieve high human chimerism, the average humanization rate is 42%⁷. In order to functionally block the murine cytochrome metabolism, a conditional (floxed exon 3 and 4) knock-out of the NADPH-P450 oxidoreductase (Por) gene was generated by targeting mouse embryonic stem cells⁸ (FIG. 4). Injected blastocysts with properly targeted embryonic stem cells generated mice with germline transmission of the Por “knock-out first” allele⁹. Expression from the targeted Por locus using the lacZ expression cassette was confirmed in the embryo and adult liver (FIG. 5). The mice were then bred with a flippase expressing strain¹⁰ to generate a CRE recombinase conditional Por knock-out strain. Homozygous zygotes from this strain were injected with the bacterial type II Clustered Regularly-Interspaced Short Palindromic Repeats/Cas9 (CRISPR-Cas9) system¹¹⁻¹³ targeting simultaneous deletion of critical exons of the Il2-rg and Rag2 and Fah gene (FIG. 6) to generate the PIRF strain (FIG. 1A). Homozygous PIRF mice are immune deficient (T-, B- and NK-cell deficient), but healthy and fertile. Since adenoviral gene therapy vectors efficiently transduce hepatocytes in vivo, the Por gene was deleted using an adenovirus coding the CRE recombinase (Adeno-CRE). Increasing doses (2.2×10⁸⁻¹⁰ per mouse) of the virus were injected intravenously into PIRF mice. Quantitative RT-PCR of the POR mRNA in liver revealed efficient deletion only at high doses (FIG. 1B). Immunostaining for POR (FIG. 1C) confirmed these findings, while a minimal residual signal could be detected by Western blotting even at the highest dose used (FIG. 1D). POR-deleted PIRF mouse livers accumulated lipids starting two weeks after adenoviral transduction (FIG. 7) similar to a previously reported liver specific Por deletion¹⁴.

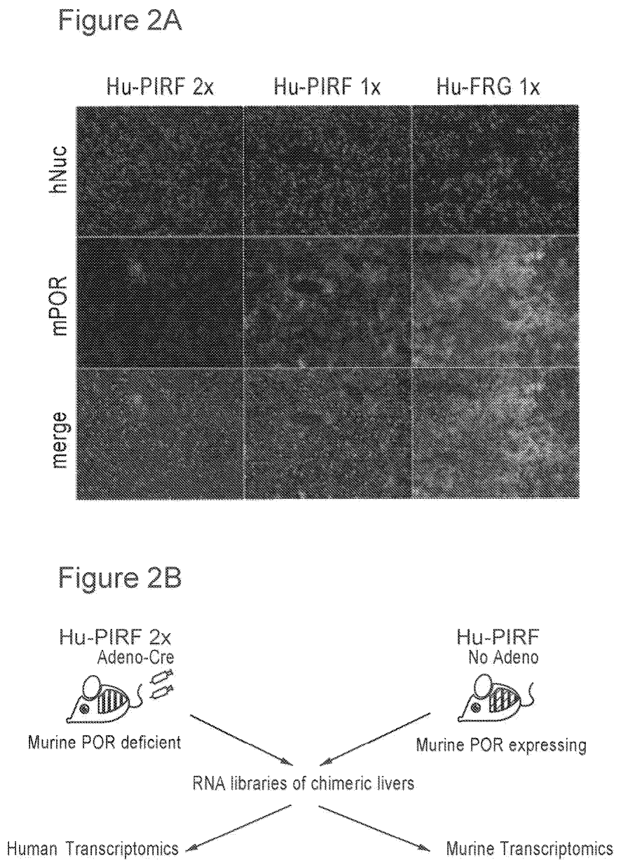

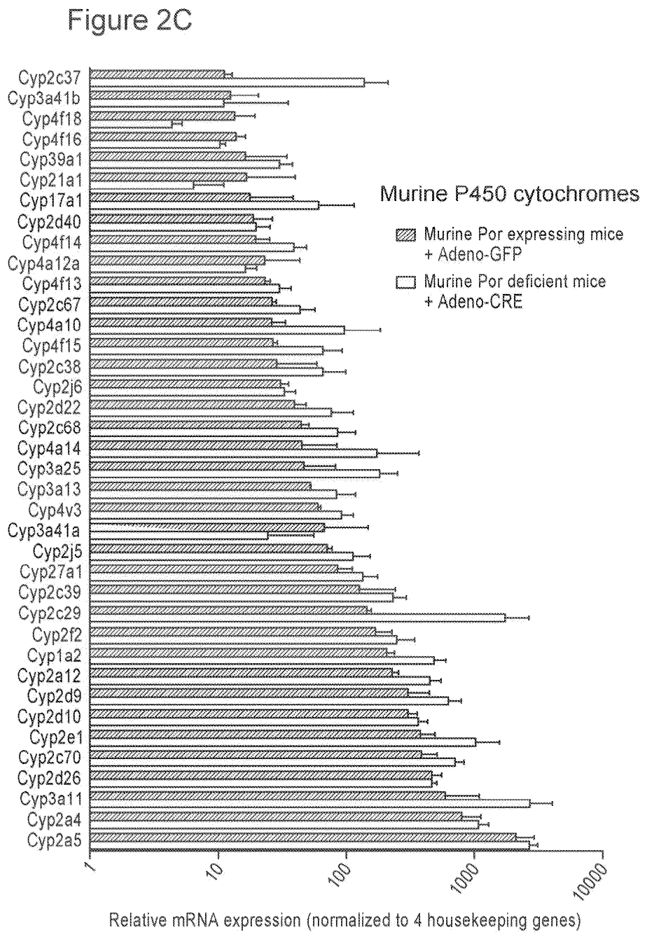

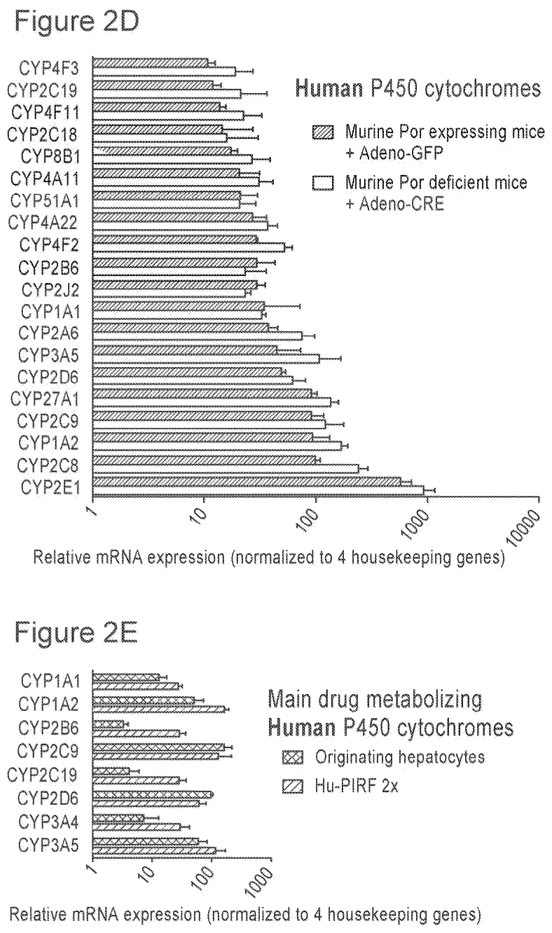

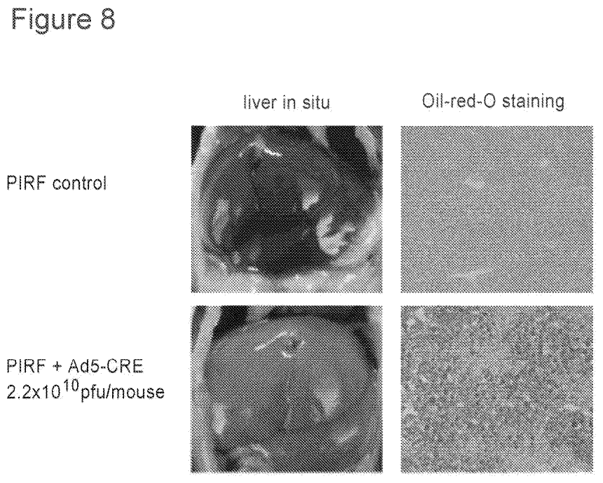

To generate human specific P450 cytochrome metabolism, human liver chimeric mice were generated by transplanting human hepatocytes^{7, 15, 16} into Por deleted PIRF mice. However, since a clonal expansion of residual Por expressing murine hepatocytes was observed in Adeno-Cre treated PIRF mice (FIG. 8), some humanized PIRF (Hu-PIRF) mice were injected with an additional dose of Adeno-Cre. Immunostaining revealed that only in double injected humanized PIRF (Hu-PIRF 2x) mice an almost complete deletion of the Por gene could be achieved (FIG. 2A).

Gene expression profiling was then performed comparing Hu-PIRF mice repopulated with the identical human hepatocytes with or without deletion of the Por (FIG. 2B). Expression of the murine P450 cytochromes was clearly altered for half of the genes: 14 cytochromes were upregulated >1.5-fold and 18 cytochromes downregulated <0.5-fold (FIG. 2c). The expression profiles of these murine cytochromes were comparable to previous work in non-humanized mice (Table 1). Table 1 shows the comparison of murine gene expression profiles of chimeric livers to previously published non-humanized mice. Gene expression of conditional (Alb-Cre) Por KO mice have been quantified by microarray analysis (Weng et al. 2005 J Biol Chem 280, 31686-31698 (2005)). Here, RNA-Seq was used to compare the gene expression (FIG. 2B) in humanized livers transduced with Adeno-Cre and Adeno-GFP. Table 1 lists all previously published cytochromes with values (fold changes) compared to the herein-described data set. Multiple numbers represent multiple sets of microarray probes.

TABLE 1
Comparison of murine gene expression
profiles of chimeric livers to previously
published non-humanized mice.

| | Change Fold (Por-deleted/ | |
|---|---|---|
| | Por non-deleted mice) | |
| Murine P450 | Present | Weng |
| Cytochromes | Disclosure | et al. 2005 |
| Cyp2a4 | 1.4 | 4.5 |
| Cyp2a5 | 1.3 | 4.5 |
| Cyp2b10 | 12.4 | 15.8/16.3/9.1 |
| Cyp2c39 | 1.8 | 1.4 |
| Cyp2c55 | 14.6 | 17.2 |
| Cyp4a10 | 3.7 | 0.3/0.7 |
| Cyp7a1 | 4.6 | 3.1/4.9 |
| Cyp7b1 | 1.6 | 0.2/0.3 |
| Cyp26a1 | 6.7 | 3.5 |
| Cyp51 | 0.6 | 2.2 |

In the same chimeric liver, all human P450 cytochromes were down regulated upon deletion of murine Por with the exception of CYP2C18 (FIG. 2D). Half of the human cytochromes were only slightly (<50%) reduced, and the other half including CYP3A4 and CYP2C19, were more significantly downregulated (>1.5-fold).

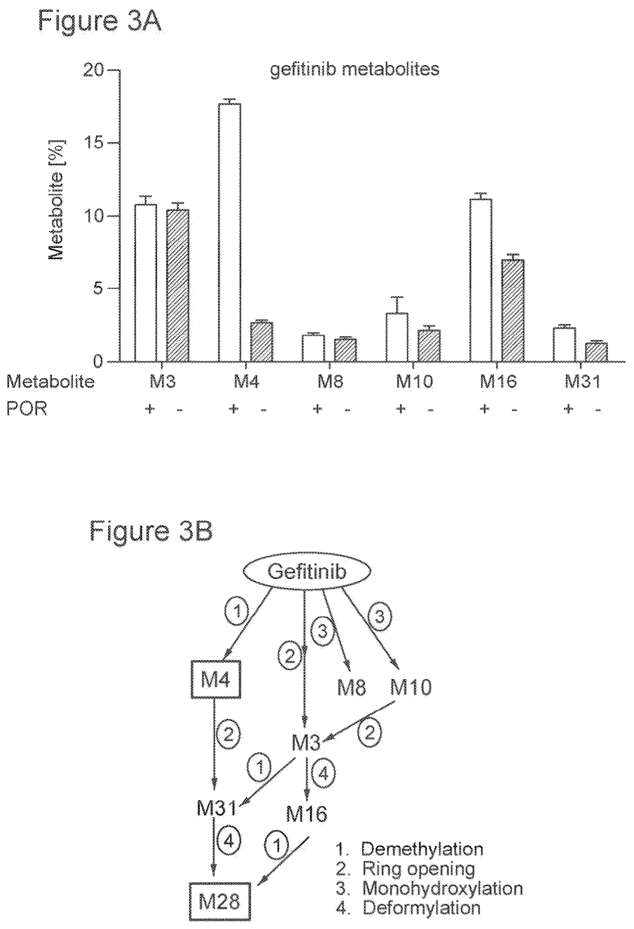

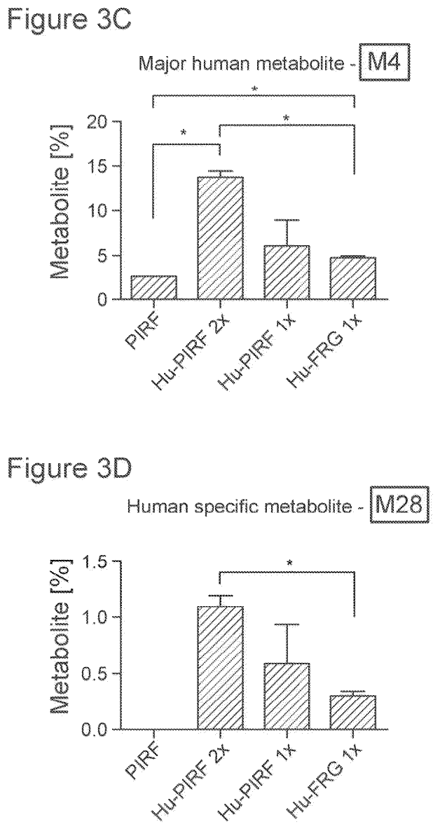

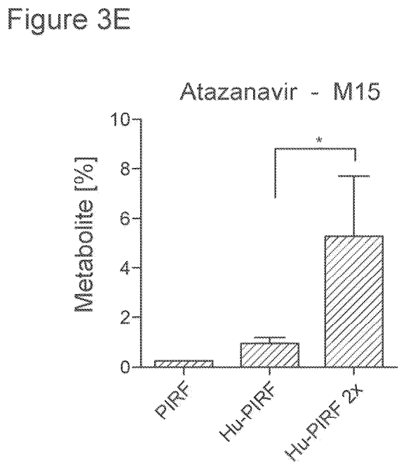

Not all human cytochromes take an important role in xenobiotic metabolism. From the 200 most transcribed drugs in the United States about three quarter are metabolized through P450 cytochromes, of which CYP3A4/5, 2C9, 2C19, 2D6 and 1A2 contribute to ˜95% of ¹⁷. These human cytochrome clusters were compared from chimeric livers (Hu-PIRF 2x) with the originating, isogenic primary hepatocytes after isolation from the donor liver. Expression levels were similar for most clusters and these important cytochromes robustly expressed in chimeric livers (FIG. 3D).

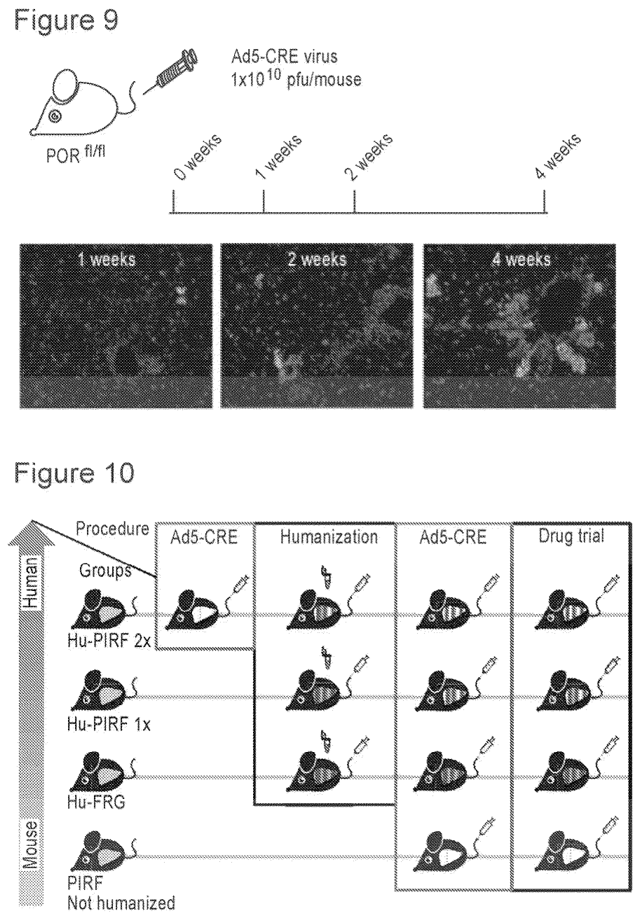

To validate utility of Hu-PIRF mice for human drug metabolism, the xenobiotic metabolism of gefetinib¹⁸, an inhibitor of epidermal growth factor receptor used against lung cancer and a variety of other cancers¹⁹, was studied. Gefetinib is primarily metabolized by the 450 cytochrome system including CYP3A4 and 2D6. New gefetinib metabolites were recently identified and demonstrated considerable differences between human and mouse liver microsomes²⁰. Gefetinib is excreted in the feces and less than 7% in the urine, irrespectively of dose, route or species^{21, 22}. Therefore, the feces of non-humanized PIRF mice was analyzed for gefetinib metabolites during the first 24-hours after intravenous injection of gefetinib. Mass spectrometry revealed a reduction of several gefetinib metabolites upon deletion of the Por gene, implying a Por-dependent P450 cytochrome deficiency for these metabolites (FIG. 3A). The biggest and most relevant reduction was observed for O-desmethyl gefitinib (M4, M523595), which is by far the most abundant metabolite in human feces while rodents produce many different metabolites including M421, 22 (FIG. 3B). Therefore, the M4 metabolite was analyzed in murine Por-deleted and Por-expressing humanized and non-humanized control mice (FIG. 9). The highest level of M4 could be detected in murine Por-deficient Hu-PIRF mice, where human hepatocytes preferentially metabolize gefitinib to M4 and remaining murine hepatocytes are inhibited in their drug metabolism (FIG. 3C). Although murine hepatocytes preferentially produce other metabolites than M4, human specific metabolites were measured. M28 was the most abundant human metabolite, which could not be detected in the non-humanized control mice. Mass spectrometry again showed the highest level of this human specific metabolite in murine Por-deficient Hu-PIRF mice confirming a more human like metabolism in these mice (FIG. 3D). Human xenobiotic metabolism was also determined with another drug, however, this time using liver homogenates of PIRF mice. Using human and mouse microsomes, it was previously demonstrated that atazanavir metabolite M15 is a predominately human metabolite (See, Li, F et al., “CYP3A-mediated generation of aldehyde and hydrazine in atazanavir metabolism.” Drug Metab Dispos 39, 394-401. Mice were intravenously injected with the retroviral therapeutic and livers were harvested 30 min after injection. Results showed that M15 was 5.4-times elevated in POR-deleted humanized PIRF mice compared to non-deleted mice (FIG. 3E) again confirming optimized human drug metabolism in this novel mouse model.

Identification of human metabolites using current experimental animal models is a major challenge. Nevertheless, identification of reactive metabolites is crucial since they drive human drug toxicity^{23, 24}. The novel humanized mouse model of the instant disclosure inhibits murine drug metabolism without impeding on the human metabolism. Murine Por-deficient humanization can be used in combination with other repopulation models like the transgenic uPA mouse and can identify more readily human specific metabolites for a greater benefit of drug safety.

Identification of mostly human or human specific metabolites is possible with the present disclosure irrespectively of toxicity. Toxicity may be present; however this is not always the case. For instance, as shown here, gefitinib did not cause any elevation of liver enzymes, yet mainly human metabolites were identified.

The present disclosure provides a method for preparing a chimeric mouse substantially lacking murine hepatocytes and instead comprising human hepatocytes, comprising steps of: (a) providing a mouse comprising a knockout mutation in each of the Il2-rg, Rag2, and Fah genes and a floxed allele of the NADPH-P450 oxidoreductase (Por) gene with a first dose of a virus that encodes Cre recombinase, thereby producing a conditional knockout of the Por gene or the knockout of the por gene using somatic genome engineering (CRIPSR/Cas9) and gene therapy vectors in Il2-rg, Rag2, and Fah deficient mice; (b) transplanting human hepatocytes into the mouse; and (c) providing the mouse with a second dose of the virus that encodes Cre recombinase. Steps (a) and (b) can occur sequentially or simultaneously.

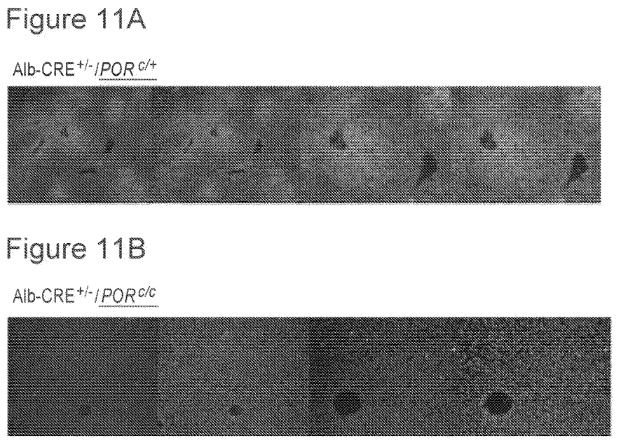

The conditional knock-out POR alleles can also be generated by delivering CRE recombinase in any way known in the art. Non-limiting examples of Cre recombinase delivery include viral or non-viral gene therapy vectors. In one embodiment, the gene therapy vector is an adenovirus. Also considered are genetic delivery of Cre recombination, e.g., under a cell-, tissue-, or developmental-specific promoter or under an inducible promoter. Indeed, Cre recombinase can be activated in the murine liver in a transgenic animal with Cre expressed under the albumin or other liver specific promoter (FIG. 11).

The present disclosure also provides a chimeric mouse, offspring thereof, or a portion thereof, which has a chimeric liver comprising human hepatocytes. Preferably, the chimeric mouse, offspring thereof or a portion thereof is prepared by the methods of the present disclosure. The chimeric mouse can be immunodeficient.

In the present disclosure, examples of the chimeric mouse include portions of the mouse. The term “a portion(s) of the mouse” refers to, mouse-derived tissues, body fluids, cells, and disrupted products thereof or extracts therefrom, for example (the examples thereof are not particularly limited to them). Examples of such tissues include, but are not particularly limited to, heart, lungs, kidney, liver, gallbladder, pancreas, spleen, intestine, muscle, blood vessel, brain, testis, ovary, uterus, placenta, marrow, thyroid gland, thymus gland, and mammary gland. Examples of body fluids include, but are not particularly limited to, blood, lymph fluids, and urine. The term “cells” refers to cells contained in the above tissues or body fluids, and examples thereof include cultured cells, sperm cells, ova, and fertilized eggs obtained by isolation or culture thereof. Examples of cultured cells include both primary cultured cells and cells of an established cell line. Examples of the portions of the mouse also include tissues, body fluids, and cells at the developmental stage (embryonic stage), as well as the disrupted products or extracts thereof. In addition, an established cell line from the mouse of the present disclosure can be established using a known method (Primary Culture Methods for Embryonic Cells (Shin Seikagaku Jikken Koza (New Biochemical Experimental Lecture Series), Vol. 18, pages 125-129, TOKYO KAGAKU DOZIN CO., LTD., and Manuals for. Mouse Embryo Manipulation, pages 262-264, Kindai Shuppan)).

The mouse of the present disclosure can be an immunodeficient mouse. The immunodeficient mouse of the present disclosure can be used as a host mouse for transplantation of human hepatocytes. Examples of the “immunodeficient mouse” may be any mouse that does not exhibit rejection against hepatocytes (in particular, human hepatocytes) from a different animal origin, and include, but are not limited to, SCID (severe combined immunodeficiency) mice exhibiting deficiency in T-and B-cell lines, mice (NUDE mice) that have lost T cell functions because of genetic deletion of the thymus gland, and mice (RAG2 knockoutmice) produced by knocking out the RAG2 gene by a known gene targeting method (Science, 244:1288-1292, 1989).

Moreover, the present disclosure provides a chimeric mouse having human hepatocytes. The chimeric mouse of the present disclosure can be immunologically deficient. The chimeric mouse of the present disclosure can be prepared by transplanting human hepatocytes into an immunodeficient mouse of the present disclosure.

As human hepatocytes to be used for transplantation, human hepatocytes isolated from normal human liver tissue by a conventional method such as a collagenase perfusion method can be used. The thus separated hepatocytes can also be used by thawing after cryopreservation. Alternatively, the chimeric mouse hepatocytes, which are defined as the human hepatocytes separated by a technique such as a collagenase perfusion method from a chimeric mouse liver, in which mouse hepatocytes have been replaced by human hepatocytes, can be used in a fresh state, and the cryopreserved chimeric mouse hepatocytes are also available after thawing.

Such human hepatocytes can be transplanted into the liver via the spleen of a mouse of the present disclosure. Such human hepatocytes can also be directly transplanted via the portal vein. The number of human hepatocytes to be transplanted may range from about 1 to 2,000,000 cells and preferably range from about 200,000 to 1,000,000 cells. The gender of the mouse of the present disclosure is not particularly limited. Also, the age on days of the mouse of the present disclosure upon transplantation is not particularly limited. When human hepatocytes are transplanted into a young mouse (early weeks of age), human hepatocytes can more actively proliferate as the mouse grows. Hence, about 0- to 40-day-old mice after birth, and particularly about 8-to 40-day-old mice after birth are preferably used.

The transplanted human hepatocytes account for any percentage of human chimerism greater than about 1%, for example at least 5%, at least 10%, at least 20%, at least 30%, at least 40%, at least 50%, at least 60%, at least 70%, at least 80%, at least 90%, at least 95% or at least 99% of all hepatocytes in the chimeric liver of the chimeric non-human animal.

The present disclosure further provides a method for screening for a substance that affects human liver functions, with the use of the chimeric mouse of the present disclosure. An example of the method is an evaluation method comprising the following steps of: (a) administering a test substance to the chimeric mouse of the present disclosure; (b) measuring one or more values in the chimeric mouse to which the test substance is administered in (a); and (c) selecting a test substance that causes an increase or an decrease in one or more values measured in (b), compared with the one or more values of the chimeric mouse to which no test substance is administered.

Preferably, the one or more values are selected from the group consisting of the human albumin concentration, the body weight curve, the liver-weight-to-body-weight ratio, the total albumin level, the total protein level, the ALT level, the AST level, and the total bilirubin level, histological assessment for toxicity in the human and non-human organs.

Examples of the “test substance” in the method of the present disclosure are not particularly limited and include natural compounds, organic compounds, inorganic compounds, proteins, antibodies, peptides, and single compounds such as an amino acid, and nucleic acids, as well as compound libraries, expression products from gene libraries, cell extracts, cell culture supernatants, products of fermenting microorganisms, extracts from marine creatures, plant extracts, extracts from prokaryotic cells, extracts from eukaryotic single cells, and extracts from animal cells. These products may be purified products or crude products such as plant, animal, or microbial extracts. Also, a method for producing a test substance is not particularly limited. A test substance to be used herein may be a substance isolated from a natural product, synthesized chemically or biochemically, or prepared by genetic engineering techniques.

The above test substance can be adequately labeled and then used as necessary. Examples of labels include radiolabels and fluorescent labels. Examples of the test substance include, in addition to the above test samples, mixtures of a plurality of types of these test samples.

Examples of test samples include and are not limited to feces, urine, blood (and any blood product, e.g., whole blood, serum, and plasma), and tissue, e.g., liver tissue. Liver tissue may be derived from a sample of a liver (e.g., a biopsy or explant) or may be derived from a whole, intact liver, e.g., that has been harvested after a mouse has been sacrificed.

Examples of a method for administering a test substance to mice are not particularly limited. Such an administration method can be adequately selected from among oral administration or parenteral administration such as subcutaneous, intravenous, local, transdermal, and enteral (intrarectal) administration, depending on the type of a test substance to be administered.

The present disclosure further provides a method for evaluating hepatotoxicity of a test substance against human hepatocytes, with the use of the chimeric mouse of the present disclosure. An example of this method is an evaluation method comprising the following steps of: (a) administering a test substance to the chimeric mouse of the present disclosure; (b) measuring one or more values in the chimeric mouse to which the test substance is administered in (a); and (c) evaluating the effect of the test substance on human hepatocytes using one or more indicators measured in (b), compared with the one or more indicators of the chimeric mouse to which no test substance is administered.

Preferably, the one or more values are selected from the group consisting of the human albumin concentration, the body weight curve, the liver-weight-to-body-weight ratio, the total albumin level, the total protein level, the ALT level, the AST level, and the total bilirubin level. Preferably, the one or more indicators are selected from the group consisting of an increase or a decrease in any one or more of the human albumin concentration, the body weight curve, the liver-weight-to-body-weight ratio, the total albumin level, the total protein level, the ALT level, the AST level, and the total bilirubin level.

| A human nucleic sequence encoding an exemplary Por gene of the disclosure consist or | |
|---|---|
| comprises, Genbank Accession number: NM_000941.2: | |
| (SEQ ID NO: 24) | |
| 1 | gaaggcggtg gtagcgcctc agtggtgtgg gcctgagccc tgcccaggtg cccgcagaga |
| 61 | gcagccgggc tgccagcgtt tcatgatcaa catgggagac tcccacgtgg acaccagctc |
| 121 | caccgtgtcc gaggcggtgg ccgaagaagt atctcttttc agcatgacgg acatgattct |
| 181 | gttttcgctc atcgtgggtc tcctaaccta ctggttcctc ttcagaaaga aaaaagaaga |
| 241 | agtccccgag ttcaccaaaa ttcagacatt gacctcctct gtcagagaga gcagctttgt |
| 301 | ggaaaagatg aagaaaacgg ggaggaacat catcgtgttc tacggctccc agacggggac |
| 361 | tgcagaggag tttgccaacc gcctgtccaa ggacgcccac cgctacggga tgcgaggcat |
| 421 | gtcagcggac cctgaggagt atgacctggc cgacctgagc agcctgccag agatcgacaa |
| 481 | cgccctggtg gttttctgca tggccaccta cggtgaggga gaccccaccg acaatgccca |
| 541 | ggacttctac gactggctgc aggagacaga cgtggatctc tctggggtca agttcgcggt |
| 601 | gtttggtctt gggaacaaga cctacgagca cttcaatgcc atgggcaagt acgtggacaa |
| 661 | gcggctggag cagctcggcg cccagcgcat ctttgagctg gggttgggcg acgacgatgg |
| 721 | gaacttggag gaggacttca tcacctggcg agagcagttc tggccggccg tgtgtgaaca |
| 781 | ctttggggtg gaagccactg gcgaggagtc cagcattcgc cagtacgagc ttgtggtcca |
| 841 | caccgacata gatgcggcca aggtgtacat gggggagatg ggccggctga agagctacga |
| 901 | gaaccagaag cccccctttg atgccaagaa tccgttcctg gctgcagtca ccaccaaccg |
| 961 | gaagctgaac cagggaaccg agcgccacct catgcacctg gaattggaca tctcggactc |
| 1021 | caaaatcagg tatgaatctg gggaccacgt ggctgtgtac ccagccaacg actctgctct |
| 1081 | cgtcaaccag ctgggcaaaa tcctgggtgc cgacctggac gtcgtcatgt ccctgaacaa |
| 1141 | cctggatgag gagtccaaca agaagcaccc attcccgtgc cctacgtcct accgcacggc |
| 1201 | cctcacctac tacctggaca tcaccaaccc gccgcgtacc aacgtgctgt acgagctggc |
| 1261 | gcagtacgcc tcggagccct cggagcagga gctgctgcgc aagatggcct cctcctccgg |
| 1321 | cgagggcaag gagctgtacc tgagctgggt ggtggaggcc cggaggcaca tcctggccat |
| 1381 | cctgcaggac tgcccgtccc tgcggccccc catcgaccac ctgtgtgagc tgctgccgcg |
| 1441 | cctgcaggcc cgctactact ccatcgcctc atcctccaag gtccacccca actctgtgca |
| 1501 | catctgtgcg gtggttgtgg agtacgagac caaggctggc cgcatcaaca agggcgtggc |
| 1561 | caccaactgg ctgcgggcca aggagcctgc cggggagaac ggcggccgtg cgctggtgcc |
| 1621 | catgttcgtg cgcaagtccc agttccgcct gcccttcaag gccaccacgc ctgtcatcat |
| 1681 | ggtgggcccc ggcaccgggg tggcaccctt cataggcttc atccaggagc gggcctggct |
| 1741 | gcgacagcag ggcaaggagg tgggggagac gctgctgtac tacggctgcc gccgctcgga |
| 1801 | tgaggactac ctgtaccggg aggagctggc gcagttccac agggacggtg cgctcaccca |
| 1861 | gctcaacgtg gccttctccc gggagcagtc ccacaaggtc tacgtccagc acctgctaaa |
| 1921 | gcaagaccga gagcacctgt ggaagttgat cgaaggcggt gcccacatct acgtctgtgg |
| 1981 | ggatgcacgg aacatggcca gggatgtgca gaacaccttc tacgacatcg tggctgagct |
| 2041 | cggggccatg gagcacgcgc aggcggtgga ctacatcaag aaactgatga ccaagggccg |
| 2101 | ctactccctg gacgtgtgga gctaggggcc tgcctgcccc acccacccca cagactccgg |
| 2161 | cctgtaatca gctctcctgg ctccctcccg tagtctcctg ggtgtgtttg gcttggcctt |
| 2221 | ggcatgggcg caggcccagt gacaaagact cctctgggcc tggggtgcat cctcctcagc |
| 2281 | ccccaggcca ggtgaggtcc accggcccct ggcagcacag cccagggcct gcatgggggc |
| 2341 | accgggctcc atgcctctgg aggcctctgg ccctcggtgg ctgcacagaa gggctctttc |
| 2401 | tctctgctga gctgggccca gcccctccac gtgatttcca gtgagtgtaa ataattttaa |
| 2461 | ataacctctg gcccttggaa taaagttctg ttttctgtaa aaaaaaaaa |
| The corresponding human amino acid sequence encoding an exemplary Por gene of the | |
| disclosure consist or comprises, Genbank Accession number: NP_000932.3: | |
| (SEQ ID NO: 25) | |
| 1 | minmgdshvd tsstvseava eevslfsmtd milfslivgl ltywflfrkk keevpeftki |
| 61 | qtltssvres sfvekmkktg rniivfygsq tgtaeefanr lskdahrygm rgmsadpeey |
| 121 | dladlsslpe idnalvvfcm atygegdptd naqdfydwlq etdvdlsgvk favfglgnkt |
| 181 | yehfnamgky vdkrleqlga qrifelglgd ddgnleedfi twreqfwpav cehfgveatg |
| 241 | eessirqyel vvhtdidaak vymgemgrlk syenqkppfd aknpflaavt tnrklnqgte |
| 301 | rhlmhleldi sdskiryesg dhvavypand salvnqlgki lgadldvvms lnnldeesnk |
| 361 | khpfpcptsy rtaltyyldi tnpprtnvly elaqyaseps eqellrkmas ssgegkelyl |
| 421 | swvvearrhi lailqdcpsl rppidhlcel lprlqaryys iassskvhpn svhicavvve |
| 481 | yetkagrink gvatnwlrak epagenggra lvpmfvrksq frlpfkattp vimvgpgtgv |
| 541 | apfigfiqer awlrqqgkev getllyygcr rsdedylyre elaqfhrdga ltqlnvafsr |
| 601 | eqshkvyvqh llkqdrehlw klieggahiy vcgdarnmar dvqntfydiv aelgamehaq |
| 661 | avdyikklmt kgrysldvws |
| A murine nucleic sequence encoding an exemplary Por gene of the disclosure consist or | |
| comprises, Genbank Accession number: NM_008898.2: | |
| (SEQ ID NO: 26) | |
| 1 | gggccgtggt agcgcctcag tggtgcgggc ttgcgtccgg ccccagtgcc tcagagacct |
| 61 | acaggaccgc gcgcggtgtg tgatctggtc ggtaccgagg agcgcaggtt gtgtcaccaa |
| 121 | catgggggac tctcacgaag acaccagtgc cacagtgcct gaggcagtgg ctgaagaagt |
| 181 | gtctctattc agcacaacgg acattgttct gttttctctc atcgtggggg tcctgaccta |
| 241 | ctggttcatc tttaaaaaga agaaagaaga gataccggag ttcagcaaga tccagacaac |
| 301 | ggccccacct gtcaaagaga gcagcttcgt ggaaaagatg aagaaaacgg gaaggaacat |
| 361 | tattgtattc tatggctccc agacgggaac cgcggaggag tttgccaacc ggctgtccaa |
| 421 | ggatgcccac cgctatggga tgcggggcat gtctgcagac cctgaagagt atgacttggc |
| 481 | cgacctgagc agcctgcctg agatcgacaa gtccctggta gtcttctgca tggccacata |
| 541 | cggagaaggc gaccccaccg acaacgcgca ggacttctat gattggctgc aggagactga |
| 601 | cgtggacctc acgggtgtca agtttgctgt gtttggtctc gggaacaaga cctatgagca |
| 661 | cttcaacgcc atgggcaagt atgtggacca gcggctggag cagcttggcg cccagcgaat |
| 721 | ctttgagttg ggccttggtg atgacgacgg gaacttggaa gaggatttca tcacatggag |
| 781 | ggagcagttc tggccagctg tgtgcgagtt cttcggggtg gaagccactg gggaggagtc |
| 841 | gagcatccgc cagtacgagc tcgtggtcca cgaagacatg gacacagcca aggtgtacac |
| 901 | gggtgagatg ggccgtctga agagctacga gaaccagaaa ccccccttcg atgccaagaa |
| 961 | tccattcctg gctgctgtca ccacgaaccg gaagctgaac caaggcactg agaggcatct |
| 1021 | aatgcacctg gaattggaca tctcagactc caagatcagg tatgaatctg gagatcacgt |
| 1081 | ggctgtgtac ccagccaacg actccaccct ggtcaaccag attggggaga tcctgggggc |
| 1141 | tgacctggat gtcatcatgt ctctaaacaa tctcgatgag gagtcgaata agaagcatcc |
| 1201 | gttcccctgc cccaccacct accgcacggc cctcacctac tacctggaca tcactaaccc |
| 1261 | gccacgaacc aacgtgctct acgagctggc ccagtacgcc tcagagccct cggagcagga |
| 1321 | acacctgcac aagatggcgt cctcctccgg cgagggcaag gagctgtacc tgagctgggt |
| 1381 | ggtggaggcc cggaggcaca tcctagccat tctccaagac tacccgtccc tgcggccacc |
| 1441 | catcgaccac ctgtgcgagc tcctcccgag gctgcaggcc cgctactatt ccattgcctc |
| 1501 | gtcgtctaag gtccacccca actccgtgca catctgcgcc gtggctgtgg agtatgaagc |
| 1561 | gaagtctgga cgagtgaaca agggggtggc caccagctgg cttcggacca aggaaccagc |
| 1621 | aggagagaat ggccgccggg ccctggtccc catgttcgtc cgcaagtccc agttccgctt |
| 1681 | gcctttcaag cccaccacac ctgttatcat ggtgggcccc ggcactgggg ttgccccttt |
| 1741 | catgggcttc atccaggagc gggcttggct tcgagagcaa ggcaaggagg tcggagagac |
| 1801 | gctgctctac tacggctgcc ggcgctcgga tgaggactat ctgtaccgcg aggagctggc |
| 1861 | gcgcttccac aaggacggcg ccctcacgca gcttaatgtg gccttttccc gtgagcaggc |
| 1921 | ccacaaggtc tatgttcagc acctgctcaa gagggacaaa gagcacctgt ggaagctgat |
| 1981 | ccacgaaggt ggtgcccaca tctatgtctg cggggatgct cgaaatatgg ccaaagatgt |
| 2041 | gcagaacaca ttctatgaca tcgtggccga gtttgggccc atggagcaca cccaggctgt |
| 2101 | ggactatgtt aagaagctca tgaccaaggg ccgctactcg ctggatgtat ggagctagga |
| 2161 | gctgccgccc cccacccctc gctccctgta atcacgtcct taacttcctt ctgccgacct |
| 2221 | ccacctctgg tggttcctgc cctgcctgga cacagggagg cccagggact gactcctggc |
| 2281 | ctgagtgatg ccctcctggg cccttaggca gagcctggtc cattgtacca ggcagcctag |
| 2341 | cccagcccag ggcacatggc aagagggact ggacccacct ttgggtgatg ggtgccttag |
| 2401 | gtccccagca gctgtacaga aggggctctt ctctccacag agctggggtg cagccccaac |
| 2461 | atgtgatttt gaatgagtgt aaataatttt aaataacctg gcccttggaa taaagttgtt |
| 2521 | ttctgta |
| The corresponding murine amino acid sequence encoding an exemplary Por gene of the | |
| disclosure consist or comprises, Genbank Accession number: NP_032924.1: | |
| (SEQ ID NO: 27) | |
| 1 | mgdshedtsa tvpeavaeev slfsttdivl fslivgvlty wfifkkkkee ipefskiqtt |
| 61 | appvkessfv ekmkktgrni ivfygsqtgt aeefanrlsk dahrygmrgm sadpeeydla |
| 121 | dlsslpeidk slvvfcmaty gegdptdnaq dfydwlqetd vdltgvkfav fglgnktyeh |
| 181 | fnamgkyvdq rleqlgaqri felglgdddg nleedfitwr eqfwpavcef fgveatgees |
| 241 | sirqyelvvh edmdtakvyt gemgrlksye nqkppfdakn pflaavttnr klnqgterhl |
| 301 | mhleldisds kiryesgdhv avypandstl vnqigeilga dldvimslnn ldeesnkkhp |
| 361 | fpcpttyrta ltyylditnp prtnvlyela qyasepseqe hlhkmasssg egkelylswv |
| 421 | vearrhilai lqdypslrpp idhlcellpr lqaryysias sskvhpnsvh icavaveyea |
| 481 | ksgrvnkgva tswlrtkepa gengrralvp mfvrksqfrl pfkpttpvim vgpgtgvapf |
| 541 | mgfiqerawl reqgkevget llyygcrrsd edylyreela rfhkdgaltq lnvafsreqa |
| 601 | hkvyvqhllk rdkehlwkli heggahiyvc gdarnmakdv qntfydivae fgpmehtqav |
| 661 | dyvkklmtkg rysldvws |
| A human nucleic sequence encoding an exemplary II2-rg gene of the disclosure consist or | |
| comprises, Genbank Accession number: NM_000206.2: | |
| (SEQ ID NO: 28) | |
| 1 | agaggaaacg tgtgggtggg gaggggtagt gggtgaggga cccaggttcc tgacacagac |
| 61 | agactacacc cagggaatga agagcaagcg ccatgttgaa gccatcatta ccattcacat |
| 121 | ccctcttatt cctgcagctg cccctgctgg gagtggggct gaacacgaca attctgacgc |
| 181 | ccaatgggaa tgaagacacc acagctgatt tcttcctgac cactatgccc actgactccc |
| 241 | tcagtgtttc cactctgccc ctcccagagg ttcagtgttt tgtgttcaat gtcgagtaca |
| 301 | tgaattgcac ttggaacagc agctctgagc cccagcctac caacctcact ctgcattatt |
| 361 | ggtacaagaa ctcggataat gataaagtcc agaagtgcag ccactatcta ttctctgaag |
| 421 | aaatcacttc tggctgtcag ttgcaaaaaa aggagatcca cctctaccaa acatttgttg |
| 481 | ttcagctcca ggacccacgg gaacccagga gacaggccac acagatgcta aaactgcaga |
| 541 | atctggtgat cccctgggct ccagagaacc taacacttca caaactgagt gaatcccagc |
| 601 | tagaactgaa ctggaacaac agattcttga accactgttt ggagcacttg gtgcagtacc |
| 661 | ggactgactg ggaccacagc tggactgaac aatcagtgga ttatagacat aagttctcct |
| 721 | tgcctagtgt ggatgggcag aaacgctaca cgtttcgtgt tcggagccgc tttaacccac |
| 781 | tctgtggaag tgctcagcat tggagtgaat ggagccaccc aatccactgg gggagcaata |
| 841 | cttcaaaaga gaatcctttc ctgtttgcat tggaagccgt ggttatctct gttggctcca |
| 901 | tgggattgat tatcagcctt ctctgtgtgt atttctggct ggaacggacg atgccccgaa |
| 961 | ttcccaccct gaagaaccta gaggatcttg ttactgaata ccacgggaac ttttcggcct |
| 1021 | ggagtggtgt gtctaaggga ctggctgaga gtctgcagcc agactacagt gaacgactct |
| 1081 | gcctcgtcag tgagattccc ccaaaaggag gggcccttgg ggaggggcct ggggcctccc |
| 1141 | catgcaacca gcatagcccc tactgggccc ccccatgtta caccctaaag cctgaaacct |
| 1201 | gaaccccaat cctctgacag aagaacccca gggtcctgta gccctaagtg gtactaactt |
| 1261 | tccttcattc aacccacctg cgtctcatac tcacctcacc ccactgtggc tgatttggaa |
| 1321 | ttttgtgccc ccatgtaagc accccttcat ttggcattcc ccacttgaga attacccttt |
| 1381 | tgccccgaac atgtttttct tctccctcag tctggccctt ccttttcgca ggattcttcc |
| 1441 | tccctccctc tttccctccc ttcctctttc catctaccct ccgattgttc ctgaaccgat |
| 1501 | gagaaataaa gtttctgttg ataatcatca aaaaaaaaaa aaaaaaaaaa aaaaaaaaaa |
| The corresponding human amino acid sequence encoding an exemplary II2-rg gene of the | |
| disclosure consist or comprises, Genbank Accession number: NP_000197.1: | |
| (SEQ ID NO: 29) | |
| 1 | mlkpslpfts llflqlpllg vglnttiltp ngnedttadf flttmptdsl svstlplpev |
| 61 | qcfvfnveym nctwnsssep qptnltlhyw yknsdndkvq kcshylfsee itsgcqlqkk |
| 121 | eihlyqtfvv qlqdpreprr qatqmlklqn lvipwapenl tlhklsesql elnwnnrfln |
| 181 | hclehlvqyr tdwdhswteq svdyrhkfsl psvdgqkryt frvrsrfnpl cgsaqhwsew |
| 241 | shpihwgsnt skenpflfal eavvisvgsm gliisllcvy fwlertmpri ptlknledlv |
| 301 | teyhgnfsaw sgvskglaes lqpdyserlc lvseippkgg algegpgasp cnqhspywap |
| 361 | pcytlkpet |
| A murine nucleic sequence encoding an exemplary II2-rg gene of the disclosure consist | |
| or comprises, Genbank Accession number: NM_013563.4: | |
| (SEQ ID NO: 30) | |
| 1 | aggaaatgta tgggtgggga gggcttgtgg gagagtggtt cagggttctg acacagacta |
| 61 | cacccagaga aagaagagca agcaccatgt tgaaactatt attgtcacct agatccttct |
| 121 | tagtccttca gctgctcctg ctgagggcag ggtggagctc caaggtcctc atgtccagtg |
| 181 | cgaatgaaga catcaaagct gatttgatcc tgacttctac agcccctgaa cacctcagtg |
| 241 | ctcctactct gccccttcca gaggttcagt gctttgtgtt caacatagag tacatgaatt |
| 301 | gcacttggaa tagcagttct gagcctcagg caaccaacct cacgctgcac tataggtaca |
| 361 | aggtatctga taataataca ttccaggagt gcagtcacta tttgttctcc aaagagatta |
| 421 | cttctggctg tcagatacaa aaagaagata tccagctcta ccagacattt gttgtccagc |
| 481 | tccaggaccc ccagaaaccc cagaggcgag ctgtacagaa gctaaaccta cagaatcttg |
| 541 | tgatcccacg ggctccagaa aatctaacac tcagcaatct gagtgaatcc cagctagagc |
| 601 | tgagatggaa aagcagacat attaaagaac gctgtttaca atacttggtg cagtaccgga |
| 661 | gcaacagaga tcgaagctgg acggaactaa tagtgaatca tgaacctaga ttctccctgc |
| 721 | ctagtgtgga tgagctgaaa cggtacacat ttcgggttcg gagccgctat aacccaatct |
| 781 | gtggaagttc tcaacagtgg agtaaatgga gccagcctgt ccactggggg agtcatactg |
| 841 | tagaggagaa tccttccttg tttgcactgg aagctgtgct tatccctgtt ggcaccatgg |
| 901 | ggttgattat taccctgatc tttgtgtact gttggttgga acgaatgcct ccaattcccc |
| 961 | ccatcaagaa tctagaggat ctggttactg aataccaagg gaacttttcg gcctggagtg |
| 1021 | gtgtgtctaa agggctgact gagagtctgc agccagacta cagtgaacgg ttctgccacg |
| 1081 | tcagcgagat tccccccaaa ggaggggccc taggagaggg gcctggaggt tctccttgca |
| 1141 | gcctgcatag cccttactgg cctcccccat gttattctct gaagccggaa gcctgaacat |
| 1201 | caatcctttg atggaacctc aaagtcctat agtcctaagt gacgctaacc tcccctactc |
| 1261 | accttggcaa tctggatcca atgctcactg ccttcccttg gggctaagtt tcgatttcct |
| 1321 | gtcccatgta actgcttttc tgttccatat gccctacttg agagtgtccc ttgccctctt |
| 1381 | tccctgcaca agccctccca tgcccagcct aacacctttc cactttcttt gaagagagtc |
| 1441 | ttaccctgta gcccagggtg gctgggagct cactatgtag gccaggttgg cctccaactc |
| 1501 | acaggctatc ctcccacctc tgcctcataa gagttggggt tactggcatg caccaccaca |
| 1561 | cccagcatgg tccttctctt ttataggatt ctccctccct ttttctacct atgattcaac |
| 1621 | tgtttccaaa tcaacaagaa ataaagtttt taaccaatga tca |
| The corresponding murine amino acid sequence encoding an exemplary II2-rg gene of the | |
| disclosure consist of, Genbank Accession number: NP_038591.1: | |
| (SEQ ID NO: 31) | |
| 1 | mlklllsprs flvlqllllr agwsskvlms sanedikadl iltstapehl saptlplpev |
| 61 | qcfvfnieym nctwnsssep qatnltlhyr ykvsdnntfq ecshylfske itsgcqiqke |
| 121 | diglyqtfvv qlgdpqkpqr ravqklnlgn lviprapenl tlsnlsesql elrwksrhik |
| 181 | erclqylvqy rsnrdrswte livnheprfs lpsvdelkry tfrvrsrynp icgssqqwsk |
| 241 | wsqpvhwgsh tveenpslfa leavlipvgt mgliitlifv ycwlermppi ppiknledlv |
| 301 | teyqgnfsaw sgvskgltes lqpdyserfc hvseippkgg algegpggsp cslhspywpp |
| 361 | pcyslkpea |
| A human nucleic sequence encoding an exemplary Rag2 gene of the disclosure consist or | |
| comprises, Genbank Accession number: NM_000536.3: | |
| (SEQ ID NO: 32) | |
| 1 | attagatcag tgttcataag aacatctgta ggcacacata cacactctct ttacagtcag |
| 61 | ccttctgctt gccacagtca tagtgggcag tcagtgaatc ttccccaagt gctgacaatt |
| 121 | aatacctggt ttagcggcaa agattcagag aggcgtgagc agcccctctg gccttcagac |
| 181 | aaaaatctac gtaccatcag aaactatgtc tctgcagatg gtaacagtca gtaataacat |
| 241 | agccttaatt cagccaggct tctcactgat gaattttgat ggacaagttt tcttctttgg |
| 301 | acaaaaaggc tggcccaaaa gatcctgccc cactggagtt ttccatctgg atgtaaagca |
| 361 | taaccatgtc aaactgaagc ctacaatttt ctctaaggat tcctgctacc tccctcctct |
| 421 | tcgctaccca gccacttgca cattcaaagg cagcttggag tctgaaaagc atcaatacat |
| 481 | catccatgga gggaaaacac caaacaatga ggtttcagat aagatttatg tcatgtctat |
| 541 | tgtttgcaag aacaacaaaa aggttacttt tcgctgcaca gagaaagact tggtaggaga |
| 601 | tgttcctgaa gccagatatg gtcattccat taatgtggtg tacagccgag ggaaaagtat |
| 661 | gggtgttctc tttggaggac gctcatacat gccttctacc cacagaacca cagaaaaatg |
| 721 | gaatagtgta gctgactgcc tgccctgtgt tttcctggtg gattttgaat ttgggtgtgc |
| 781 | tacatcatac attcttccag aacttcagga tgggctatct tttcatgtct ctattgccaa |
| 841 | aaatgacacc atctatattt taggaggaca ttcacttgcc aataatatcc ggcctgccaa |
| 901 | cctgtacaga ataagggttg atcttcccct gggtagccca gctgtgaatt gcacagtctt |
| 961 | gccaggagga atctctgtct ccagtgcaat cctgactcaa actaacaatg atgaatttgt |
| 1021 | tattgttggt ggctatcagc ttgaaaatca aaaaagaatg atctgcaaca tcatctcttt |
| 1081 | agaggacaac aagatagaaa ttcgtgagat ggagacccca gattggaccc cagacattaa |
| 1141 | gcacagcaag atatggtttg gaagcaacat gggaaatgga actgtttttc ttggcatacc |
| 1201 | aggagacaat aaacaagttg tttcagaagg attctatttc tatatgttga aatgtgctga |
| 1261 | agatgatact aatgaagagc agacaacatt cacaaacagt caaacatcaa cagaagatcc |
| 1321 | aggggattcc actccctttg aagactctga agaattttgt ttcagtgcag aagcaaatag |
| 1381 | ttttgatggt gatgatgaat ttgacaccta taatgaagat gatgaagaag atgagtctga |
| 1441 | gacaggctac tggattacat gctgccctac ttgtgatgtg gatatcaaca cttgggtacc |
| 1501 | attctattca actgagctca acaaacccgc catgatctac tgctctcatg gggatgggca |
| 1561 | ctgggtccat gctcagtgca tggatctggc agaacgcaca ctcatccatc tgtcagcagg |
| 1621 | aagcaacaag tattactgca atgagcatgt ggagatagca agagctctac acactcccca |
| 1681 | aagagtccta cccttaaaaa agcctccaat gaaatccctc cgtaaaaaag gttctggaaa |
| 1741 | aatcttgact cctgccaaga aatcctttct tagaaggttg tttgattagt tttgcaaaag |
| 1801 | cctttcagat tcaggtgtat ggaatttttg aatctatttt taaaatcata acattgattt |
| 1861 | taaaaataca tttttgttta tttaaaatgc ctatgttttc ttttagttac atgaattaag |
| 1921 | ggccagaaaa aagtgtttat aatgcaatga taaataaagt cattctagac cctatacatt |
| 1981 | ttgaaaatat tttacccaaa tactcaattt actaatttat tcttcactga ggatttctga |
| 2041 | tctgattttt tattcaacaa accttaaaca cccagaagca gtaataatca tcgaggtatg |
| 2101 | tttatattta ttatataagt cttggtaaca aataacctat aaagtgttta tgacaaattt |
| 2161 | agccaataaa gaaattaaca cccaaaagaa ttaaattgat tattttgtgc aacataacaa |
| 2221 | ttcggcagtt ggccaaaact taaaagcaag atctactaca tcccacatta gtgttcttta |
| 2281 | tataccttca agcaaccctt tggattatgc ccatgaacaa gttagtttct catagcttta |
| 2341 | cagatgtaga tataaatata aatatatgta tacatataga tagataatgt tctccactga |
| 2401 | cacaaaagaa gaaataaata atctacatca aaaaaaaaaa aaaaaaaaaa aaaaaaa |
| The corresponding human amino acid sequence encoding an exemplary Rag2 gene of the | |
| disclosure consist or comprises, Genbank Accession number: NP_000527.2: | |
| (SEQ ID NO: 33) | |
| 1 | mslqmvtvsn nialiqpgfs lmnfdgqvff fgqkgwpkrs cptgvfhldv khnhvklkpt |
| 61 | ifskdscylp plrypatctf kgslesekhq yiihggktpn nevsdkiyvm sivcknnkkv |
| 121 | tfrctekdlv gdvpearygh sinvvysrgk smgvlfggrs ympsthrtte kwnsvadclp |
| 181 | cvflvdfefg catsyilpel qdglsfhvsi akndtiyilg ghslannirp anlyrirvdl |
| 241 | plgspavnct vlpggisvss ailtqtnnde fvivggyqle nqkrmicnii slednkieir |
| 301 | emetpdwtpd ikhskiwfgs nmgngtvflg ipgdnkqvvs egfyfymlkc aeddtneeqt |
| 361 | tftnsqtste dpgdstpfed seefcfsaea nsfdgddefd tyneddeede setgywitcc |
| 421 | ptcdvdintw vpfystelnk pamiycshgd ghwvhaqcmd laertlihls agsnkyycne |
| 481 | hveiaralht pqrvlplkkp pmkslrkkgs gkiltpakks flrrlfd |
| A murine nucleic sequence encoding an exemplary Rag2 gene of the disclosure consist or | |
| comprises, Genbank Accession number: NM_009020.3: | |
| (SEQ ID NO: 34) | |
| 1 | actctaccct gcagccttca gcttggcaca aactaaacag tgactcttcc ccaagtgccg |
| 61 | agtttaattc ctggcttggc cgaaaggatt cagagaggga taagcagccc ctctggcctt |
| 121 | cagtgccaaa ataagaaaga gtatttcaca tccacaagca ggaagtacac ttcatacctc |
| 181 | tctaagataa aagacctatt cacaatcaaa aatgtccctg cagatggtaa cagtgggtca |
| 241 | taacatagcc ttaattcaac caggcttctc acttatgaat tttgatggcc aagttttctt |
| 301 | ctttggccag aaaggctggc ctaagagatc ctgtcctact ggagtctttc attttgatat |
| 361 | aaaacaaaat catctcaaac tgaagcctgc aatcttctct aaagattcct gctacctccc |
| 421 | acctcttcgt tatccagcta cttgctcata caaaggcagc atagactctg acaagcatca |
| 481 | atatatcatt cacggaggga aaacaccaaa caatgagctt tccgataaga tttatatcat |
| 541 | gtctgtcgct tgcaagaata acaaaaaagt tactttccgt tgcacagaga aagacttagt |
| 601 | aggagatgtc cctgaaccca gatacggcca ttccattgac gtggtgtata gtcgagggaa |
| 661 | aagcatgggt gttctctttg gaggacgttc atacatgcct tctacccaga gaaccacaga |
| 721 | aaaatggaat agtgtagctg actgcctacc ccatgttttc ttgatagatt ttgaatttgg |
| 781 | gtgtgctaca tcatatattc tcccagaact tcaggatggg ctgtcttttc atgtttctat |
| 841 | tgccagaaac gataccgttt atattttggg aggacactca cttgccagta atatacgccc |
| 901 | tgctaacttg tatagaataa gagtggacct tcccctgggt accccagcag tgaattgcac |
| 961 | agtcttgcca ggaggaatct ctgtctccag tgcaatcctc actcaaacaa acaatgatga |
| 1021 | atttgttatt gtgggtggtt atcagctgga aaatcagaaa aggatggtct gcagccttgt |
| 1081 | ctctctaggg gacaacacga ttgaaatcag tgagatggag actcctgact ggacctcaga |
| 1141 | tattaagcat agcaaaatat ggtttggaag caacatggga aacgggacta ttttccttgg |
| 1201 | cataccagga gacaataagc aggctatgtc agaagcattc tatttctata ctttgagatg |
| 1261 | ctctgaagag gatttgagtg aagatcagaa aattgtctcc aacagtcaga catcaacaga |
| 1321 | agatcctggg gactccactc cctttgaaga ctcagaggaa ttttgtttca gtgctgaagc |
| 1381 | aaccagtttt gatggtgacg atgaatttga cacctacaat gaagatgatg aagatgacga |
| 1441 | gtctgtaacc ggctactgga taacatgttg ccctacttgt gatgttgaca tcaatacctg |
| 1501 | ggttccgttc tattcaacgg agctcaataa acccgccatg atctattgtt ctcatgggga |
| 1561 | tgggcactgg gtacatgccc agtgcatgga tttggaagaa cgcacactca tccacttgtc |
| 1621 | agaaggaagc aacaagtatt attgcaatga acatgtacag atagcaagag cattgcaaac |
| 1681 | tcccaaaaga aaccccccct tacaaaaacc tccaatgaaa tccctccaca aaaaaggctc |
| 1741 | tgggaaagtc ttgactcctg ccaagaaatc cttccttaga agactgtttg attaatttag |
| 1801 | caaaagcccc tcagactcag gtatattgct ctctgaatct actttcaatc ataaacatta |
| 1861 | ttttgatttt tgtttactga aatctctatg ttatgtttta gttatgtgaa ttaagtgctg |
| 1921 | ttgtgattta ttgttaagta taactattct aatgtgtgtt ttttaacatc ttatccagga |
| 1981 | atgtcttaaa tgagaaatgt tatacagttt tccattaagg atatcagtga taaagtatag |
| 2041 | aactcttaca ttattttgta acaatctaca tattgaatag taactaaata ccaataaata |
| 2101 | aactaatgca caaaaagtta agttcttttg tgtaataagt agcctatagt tggtttaaac |
| 2161 | agttaaaacc aacagctata tcccacacta ctgctgttta taaattttaa ggtggcctct |
| 2221 | ggtttatact tatgagcaga attatatata ttggtcaata ccatgaagaa aaatttaatt |
| 2281 | ctatatcaag ccaggcatgg tgatggtgat acatgcctgt aatcctggca cttaggaagt |
| 2341 | ggaagaagga agtttgtgag tttgatgctt gttgaggtat gaccttttgc tatgtattgt |
| 2401 | agtgtatgag ccccaagacc tgcttgaccc agagacaaga gagtccacac atagatccaa |
| 2461 | gtaatgctat gtgaccttgc cccccggtta cttgtgatta ggtgaataaa gatgtcaaca |
| 2521 | gccaatagct gggcagaaga gccaaaagtg gggattgagg gtaccctggc ttgatgtagg |
| 2581 | aggagaccat gaggaaaggg gagaaaaaag tgatggagga ggagaaagat gccatgagct |
| 2641 | aggagttaag aaagcatggc catgagtgct ggccaattgg agttaagagc agcccagatg |
| 2701 | aaacatagta agtaataact cagggttatc gatagaaaat agattttagt gccgtactct |
| 2761 | ccccagccct agagctgact atggcttact gtaaatataa agtttgtatg tgtcttttat |
| 2821 | ccaggaacta aatggtcaaa ggtggagtag aaactctgga ttgggattaa atttttctac |
| 2881 | aacaaatgct ggcctgggct agattttatc tcatatccga aggctgacag aacacagagc |
| 2941 | actggtaaca ttgccacctg ccatgcacaa agacctgagt ctaatactgt ggacattttc |
| 3001 | ttgaagtatc tacatgtact tctggagtga aaacatattc caacaatatg cctttgttta |
| 3061 | aatcactcac tcactttggg ccctcacatt atatcctttc aaaatcaatg gttcacccct |
| 3121 | ttgaaaatgc ttagccatag tccctcatct tccttaaaga cagttgtcat ctctggaaat |
| 3181 | agtcacatgt cattcaaggt ccaatactgt gcagctctga agtatggcat taccacttta |
| 3241 | agtgaaaagt gaaatatgaa catgagctca gacaaaggtt tgggactatc actctcaagg |
| 3301 | aggctctact gctaagtcct gaactgcttt cacatgaata cagaaattat aacaaaaaat |
| 3361 | atgtaatcaa taaaaagaaa actttcatat tcc |
| The corresponding amino acid sequence encoding an exemplary Rag2 gene of the | |
| disclosure consist or comprises of gene consist of, Genbank Accession number: | |
| NP_033046.1: | |
| (SEQ ID NO: 35) | |
| 1 | mslqmvtvgh nialiqpgfs lmnfdgqvff fgqkgwpkrs cptgvfhfdi kqnhlklkpa |
| 61 | ifskdscylp plrypatcsy kgsidsdkhq yiihggktpn nelsdkiyim svacknnkkv |
| 121 | tfrctekdlv gdvpeprygh sidvvysrgk smgvlfggrs ympstqrtte kwnsvadclp |
| 181 | hvflidfefg catsyilpel qdglsfhvsi arndtvyilg ghslasnirp anlyrirvdl |
| 241 | plgtpavnct vlpggisvss ailtqtnnde fvivggyqle nqkrmvcslv slgdntieis |
| 301 | emetpdwtsd ikhskiwfgs nmgngtiflg ipgdnkqams eafyfytlrc seedlsedqk |
| 361 | ivsnsqtste dpgdstpfed seefcfsaea tsfdgddefd tyneddedde svtgywitcc |
| 421 | ptcdvdintw vpfystelnk pamiycshgd ghwvhaqcmd leertlihls egsnkyycne |
| 481 | hvqiaralqt pkrnpplqkp pmkslhkkgs gkvltpakks flrrlfd |
| A human nucleic sequence encoding an exemplary Fah gene of the disclosure consist or | |
| comprises of gene consist of, Genbank Accession number: NM_000137.2: | |
| (SEQ ID NO: 36) | |
| 1 | gagaccaaaa gtcaggtagg agcctccggg gtccctgctg tgtcacccgg acaggccgtg |
| 61 | ggggcgggca ggggggcggg gccgggcctg accacagcgg ccgagttcag tcctgctctc |
| 121 | cgcacgccac cttaggcccg cagccgtgcc gggtgctctt cagcatgtcc ttcatcccgg |
| 181 | tggccgagga ttccgacttc cccatccaca acctgcccta cggcgtcttc tcgaccagag |
| 241 | gcgacccaag accgaggata ggtgtggcca ttggcgacca gatcctggac ctcagcatca |
| 301 | tcaagcacct ctttactggt cctgtcctct ccaaacacca ggatgtcttc aatcagccta |
| 361 | cactcaacag cttcatgggc ctgggtcagg ctgcctggaa ggaggcgaga gtgttcttgc |
| 421 | agaacttgct gtctgtgagc caagccaggc tcagagatga caccgaactt cggaagtgtg |
| 481 | cattcatctc ccaggcttct gccacgatgc accttccagc caccatagga gactacacag |
| 541 | acttctattc ctctcggcag catgctacca acgtcggaat catgttcagg gacaaggaga |
| 601 | atgcgttgat gccaaattgg ctgcacttac cagtgggcta ccatggccgt gcctcctctg |
| 661 | tcgtggtgtc tggcacccca atccgaaggc ccatgggaca gatgaaacct gatgactcta |
| 721 | agcctcccgt atatggtgcc tgcaagctct tggacatgga gctggaaatg gctttttttg |
| 781 | taggccctgg aaacagattg ggagagccga tccccatttc caaggcccat gagcacattt |
| 841 | ttggaatggt ccttatgaac gactggagtg cacgagacat tcagaagtgg gagtatgtcc |
| 901 | ctctcgggcc attccttggg aagagttttg ggaccactgt ctctccgtgg gtggtgccca |
| 961 | tggatgctct catgcccttt gctgtgccca acccgaagca ggaccccagg cccctgccgt |
| 1021 | atctgtgcca tgacgagccc tacacatttg acatcaacct ctctgttaac ctgaaaggag |
| 1081 | aaggaatgag ccaggcggct accatatgca agtccaattt taagtacatg tactggacga |
| 1141 | tgctgcagca gctcactcac cactctgtca acggctgcaa cctgcggccg ggggacctcc |
| 1201 | tggcttctgg gaccatcagc gggccggagc cagaaaactt cggctccatg ttggaactgt |
| 1261 | cgtggaaggg aacgaagccc atagacctgg ggaatggtca gaccaggaag tttctgctgg |
| 1321 | acggggatga agtcatcata acagggtact gccaggggga tggttaccgc atcggctttg |
| 1381 | gccagtgtgc tggaaaagtg ctgcctgctc tcctgccatc atgagatttt ctctgctctt |
| 1441 | ctggaaacaa agggctcaag cacccctttc aaccctgtga ctggggtcct ccctcgggct |
| 1501 | gtaggcctgg tccgccattc agtgacaaat aaagccattg tgctctgagg cctgcactgc |
| 1561 | cgcagatgca gctgtgtcca cttatgatcg tgatttgatc cagtgggtca aggtgtgtaa |
| 1621 | agcctccctg ccagatattc attaatatgt tttctcactc ttattagtga ggtcaggggt |
| 1681 | ctttgtggga ttttcttatt agacatccca ggcctcctgg tattccatgg aatttgaaaa |
| 1741 | gagactggca cctgtagtag tcagggctct ccagagaaat agaaccaagg agaaagaaaa |
| 1801 | aaaaaaaaaa |
| The corresponding human amino acid sequence encoding an exemplary Fah gene of the | |
| disclosure consist or comprises of gene consist of, Genbank Accession number: | |
| NP_000128.1: | |
| (SEQ ID NO: 37) | |
| 1 | msfipvaeds dfpihnlpyg vfstrgdprp rigvaigdqi ldlsiikhlf tgpvlskhqd |
| 61 | vfnqptlnsf mglgqaawke arvflqnlls vsqarlrddt elrkcafisq asatmhlpat |
| 121 | igdytdfyss rqhatnvgim frdkenalmp nwlhlpvgyh grassvvvsg tpirrpmgqm |
| 181 | kpddskppvy gacklldmel emaffvgpgn rlgepipisk ahehifgmvl mndwsardiq |
| 241 | kweyvplgpf lgksfgttvs pwvvpmdalm pfavpnpkqd prplpylchd epytfdinls |
| 301 | vnlkgegmsq aaticksnfk ymywtmlqql thhsvngcnl rpgdllasgt isgpepenfg |
| 361 | smlelswkgt kpidlgngqt rkflldgdev iitgycqgdg yrigfgqcag kvlpallps |
| (SEQ ID NO: 38) | |
| 1 | gggtgctaaa agaatcacta gggtggggag gcggtcccag tggggcgggt aggggtgtgt |
| 61 | gccaggtggt accgggtatt ggctggagga agggcagccc ggggttcggg gcggtccctg |
| 121 | aatctaaagg ccctcggcta gtctgatcct tgccctaagc atagtcccgt tagccaaccc |
| 181 | cctacccgcc gtgggctctg ctgcccggtg ctcgtcagca tgtcctttat tccagtggcc |
| 241 | gaggactccg actttcccat ccaaaacctg ccctatggtg ttttctccac tcaaagcaac |
| 301 | ccaaagccac ggattggtgt agccatcggt gaccagatct tggacctgag tgtcattaaa |
| 361 | cacctcttta ccggacctgc cctttccaaa catcaacatg tcttcgatga gacaactctc |
| 421 | aataacttca tgggtctggg tcaagctgca tggaaggagg caagagcatc cttacagaac |
| 481 | ttactgtctg ccagccaagc ccggctcaga gatgacaagg agcttcggca gcgtgcattc |
| 541 | acctcccagg cttctgcgac aatgcacctt cctgctacca taggagacta cacggacttc |
| 601 | tactcttctc ggcagcatgc caccaatgtt ggcattatgt tcagaggcaa ggagaatgcg |
| 661 | ctgttgccaa attggctcca cttacctgtg ggataccatg gccgagcttc ctccattgtg |
| 721 | gtatctggaa ccccgattcg aagacccatg gggcagatga gacctgataa ctcaaagcct |
| 781 | cctgtgtatg gtgcctgcag actcttagac atggagttgg aaatggcttt cttcgtaggc |
| 841 | cctgggaaca gattcggaga gccaatcccc atttccaaag cccatgaaca cattttcggg |
| 901 | atggtcctca tgaacgactg gagcgcacga gacatccagc aatgggagta cgtcccactt |
| 961 | gggccattcc tggggaaaag ctttggaacc acaatctccc cgtgggtggt gcctatggat |
| 1021 | gccctcatgc cctttgtggt gccaaaccca aagcaggacc ccaagccctt gccatatctc |
| 1081 | tgccacagcc agccctacac atttgatatc aacctgtctg tctctttgaa aggagaagga |
| 1141 | atgagccagg cggctaccat ctgcaggtct aactttaagc acatgtactg gaccatgctg |
| 1201 | cagcaactca cacaccactc tgttaatgga tgcaacctga gacctgggga cctcttggct |
| 1261 | tctggaacca tcagtggatc agaccctgaa agctttggct ccatgctgga actgtcctgg |
| 1321 | aagggaacaa aggccatcga tgtggagcag gggcagacca ggaccttcct gctggacggc |
| 1381 | gatgaagtca tcataacagg tcactgccag ggggacggct accgtgttgg ctttggccag |
| 1441 | tgtgctggga aagtgctgcc tgccctttca ccagcctgaa gctccggaag tcacaagaca |
| 1501 | cacccttgcc ttatgaggat catgctacca ctgcatcagt caggaatgaa taaagctact |
| 1561 | ttgattgtgg gaaatgccac agaaaaaaaa aaaaaaa |
| The corresponding murine amino acid sequence encoding an exemplary Fah gene of the | |
| disclosure consist or comprises of gene consist of, Genbank Accession number: | |
| NP_034306.2: | |
| (SEQ ID NO: 39) | |
| 1 | msfipvaeds dfpiqnlpyg vfstqsnpkp rigvaigdgi ldlsvikhlf tgpalskhqh |
| 61 | vfdettlnnf mglgqaawke araslqnlls asgarlrddk elrgraftsq asatmhlpat |
| 121 | igdytdfyss rqhatnvgim frgkenallp nwlhlpvgyh grassivvsg tpirrpmgqm |
| 181 | rpdnskppvy gacrlldmel emaffvgpgn rfgepipisk ahehifgmvl mndwsardiq |
| 241 | qweyvplgpf lgksfgttis pwvvpmdalm pfvvpnpkqd pkplpylchs qpytfdinls |
| 301 | vslkgegmsq aaticrsnfk hmywtmlqql thhsvngcnl rpgdllasgt isgsdpesfg |
| 361 | smlelswkgt kaidveggqt rtflldgdev iitghcqgdg yrvgfgqcag kvlpalspa |

The following examples are provided to better illustrate the claimed disclosure and are not to be interpreted as limiting the scope of the disclosure. To the extent that specific materials are mentioned, it is merely for purposes of illustration and is not intended to limit the disclosure. One skilled in the art may develop equivalent means or reactants without the exercise of inventive capacity and without departing from the scope of the disclosure.

### EXAMPLES

### Example 1

### Generation of the Por-Floxed Mouse Strain

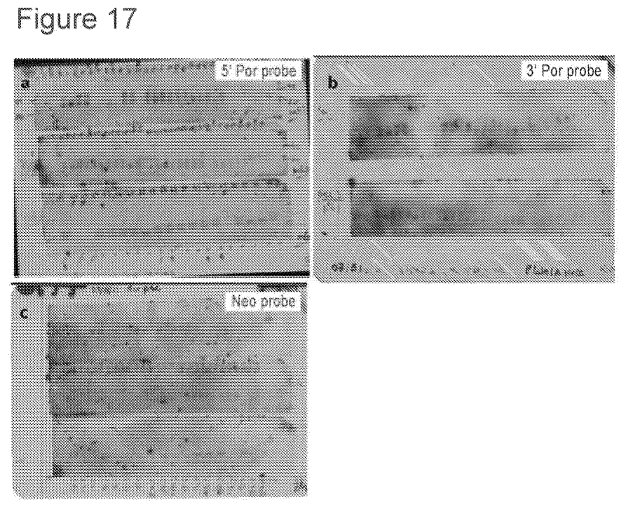

Por knock-out first targeting vector was purchased from the National Institutes of Health (NIH) Knock-Out Mouse Program (KOMP) (FIG. 4A). The vector was linearization with the AsisI restriction enzyme, and DNA was electroporated into Jm8A3 mouse embryonic stem cells (ESC) (Pettitt, S. J. et al. “Agouti C57BL/6N embryonic stem cells for mouse genetic resources.” Nat Methods 6, 493-495 (2009)) by the Mouse Embryonic Stem Cell Core at Baylor College of Medicine. Integrated clones were selected using neomycin resistance. DNA of ESC clones was digested with NSiI restriction enzyme and screened for site specific integration by Southern blotting using DIG nonisotopic detection system (Roche Applied Biosciences) following the manufacturer's instructions (full blots in FIG. 17). The 500bp-size 5′ and 3′ probes that bind outside the vector's homology arms were synthesized using the following set of primers.

5′ POR Fw2
(SEQ ID NO: 1)
GGCCTCAGAGAGGACATAGTGCCC
5′ POR Rev2
(SEQ ID NO: 2)
GCCCTCTGGTGTCAGGTCCC
3′ POR Fw2
(SEQ ID NO: 3)
CCTCACGCAGCTTAATGTGGCC
3′ POR Rev2
(SEQ ID NO: 4)
GGAAGTTAAGGACGTGATTACAGGGAGC

Correctly targeted ESCs cells were injected into C57/BL blastocysts by the Genetically Engineered Mouse Core at Baylor College of Medicine. The male chimeras were bred with C57/BL albino females (Taconic) to access germline transmission of targeted ESC. To remove the FRT-flanked LacZ and the neomycin cassette and generate a conditional POR knock-out strain, the mice were crossed with a Rosa26-FLPe strain (Farley, F. W., Soriano, P., Steffen, L. S. & Dymecki, S. M. “Widespread recombinase expression using FLPeR (flipper) mice.” Genesis 28, 106-110 (2000)). Genotyping was performed by Transnetyx (Cordova, TN).

### Example 2

### X-Gal Staining

Embryos and fresh liver sections were fixed in 4% PFA for 1 hour at 4° C. and washed 2x30min in X-Gal rinse buffer (PBS 1x with 0.02% Igepal and 0.01% deoxycholate) followed by overnight incubation with X-Gal staining solution (PBS 1x with 5 mM K₃Fe(CN)₆, 5 mM K₄Fe(CN)₆, 0.02% Igepal, 0.01% deoxycholate, 2 mM MgCl₂, 5 mM EGTA and 1 mg/ml of fresh X-Gal). Samples were post-fixed overnight in 4% PFA at 4° C.

### Example 3

### Generating of the PIRF (Por^{c-/c}-/Il2rg−/−/Rag2−/−/Fah−/−) mouse strain

Six gRNA sequences targeting critical exons of the Rag2, Il2-rg or Fah gene were selected (FIG. 1A, FIG. 6 and FIG. 7) using two different online tools (crispr.mit.edu and COSMID) (Cradick, T. J., Qiu, P., Lee, C. M., Fine, E. J. & Bao, G. “COSMID: A Web-based Tool for Identifying and Validating CRISPR/Cas Off-target Sites.” Molecular therapy. Nucleic acids 3, e214 (2014)). Complementary oligonucleotides were annealed and ligated into the DR274 vector (Addgene plasmid #42250) (Hwang, . et al. “Efficient genome editing in zebrafish using a CRISPR-Cas system.” Nat Biotechnol 31, 227-229 (2013)) using standard molecular cloning techniques with the restriction enzyme BsaI (NEB) and T4 DNA Ligase (NEB). A T7 bacterial promoter sequence was inserted into the pX330-U6-Chimeric BB-CBh-hSpCas9vector (Addgene plasmid #42230) (Cong, L. et al. “Multiplex genome engineering using CRISPR/Cas systems.” Science 339, 819-823 (2013)) upstream of the Cas9 transcription start site using standard molecular cloning techniques. DR274 vectors were cut using DraI (NEB) and gel purified using the Zymoclean Gel DNA Recovery Kit (Zymo, Cat #11-301). In vitro transcription of sgRNA was performed using the MEGAshortscript T7 Transcription Kit (Life Technologies, AM1354), according to manufacturer's instructions. The resulting RNA was purified using the RNA Clean & Concentrator-5 (Zymo, R1015) and eluted in RNAse-free water. Synthesis was verified by polyacrylamide gel electrophoresis. pX330 (with T7 promoter) was digested with NcoI and NotI, and gel purified. Cas9 mRNA was synthesized from the digested pX330-T7 vector using the mMessage mMachine T7 ULTRA Kit (life tech AM1345), according to the manufacturer's protocol. Poly-adenylation was verified by denaturing agarose gel electrophoresis (1% agarose and 6.6% formaldehyde in MOPS buffer).

Zygotes from Por c/c mice were injected with S. pyogenes Cas9 mRNA (60 ng/ul) and the six gRNA (15ng/uL each). All viable zygotes were implanted into 3 pseudopregnant females. To detect the deleted regions all twenty-three pups were genotyped after weaning using the following primers:

Fah Fw
(SEQ ID NO: 5)
CTGGGTTGCATACTGGTGGG
Fah Rev
(SEQ ID NO: 6)
AAACAGGGTCTTTGCTGCTG
Fah Int Fw
(SEQ ID NO: 7)
ACAAAGGTGTGGCAAGGGTT
Il2 Fw
(SEQ ID NO: 8)
CCACCGGAAGCTACGACAAA
Il2 Rev
(SEQ ID NO: 9)
GGGGGAATTGGAGGCATTCT
Il2 Int Rev
(SEQ ID NO: 10)
CTTCTTCCCGTGCTACCCTC
Rag2 Fw
(SEQ ID NO: 11)
CCTCCCACCTCTTCGTTATCC
Rag2 Rev
(SEQ ID NO: 12)
AGTCTGAGGGGCTTTTGCTA
Rag2 Int Fw
(SEQ ID NO: 13)
AGTCTGAGGGGCTTTTGCTA

Further offspring genotyping was performed by Transnetyx (Cordova, TN).

### Example 4

### Humanization of PIRF Mice

Hepatocytes (3×10⁶/mouse) were transplanted into the murine liver of PIRF mice by splenic injections as originally described for mouse hepatocytes (Ponder, K. P. et al. “Mouse hepatocytes migrate to liver parenchyma and function indefinitely after intrasplenic transplantation.” Proc Natl Acad Sci USA 88, 1217-1221 (1991)). In brief, the abdominal cavity was opened by a midabdominal incision, and 3×10⁶ human hepatocytes in a volume of 100 μl PBS were injected into the spleen. Immediately after transplantation, selection pressure towards transplanted human hepatocytes was applied by withdrawing the drug nitisinone (NTBC) from the drinking water in the following steps: 2 days at 25%, then 2 days at 12% and eventually 2 days at 6% of the colony maintenance dose (100%=7.5 mg/l) prior to discontinuing the drug completely (Bissig, K. D. et al. “Human liver chimeric mice provide a model for hepatitis B and C virus infection and treatment.” The Journal of clinical investigation 120, 924-930 (2010)). Mice with clinical symptoms (hunched posture, lethargy, weight loss, etc) were put back on 100% nitisinone for a few days before once again being weaned off the drug as described above. In order to determine the extent of human chimerism, human albumin (ELISA, Bethyl laboratories) in the murine blood, having previously shown that human albumin levels correlate with the level of human chimerism assessed by immunostaining of human hepatocytes was measured (Bissig, K. D. et al. (2010)). Only mice with a human chimerism >70% were further used. Where indicated, some PIRF mice were injected intravenously with 100 μl

Adenovirus coding CRE recombinase under the CMV promoter (Ad5 CMV-Cre, 2.3×10¹¹ pfu/ml, provided by the Vector Development Laboratory at Baylor College of Medicine) either 24-hours before hepatocyte transplantation and/or when reaching high human chimerism (>70%). Available hepatocyte donor information is given in Table 2. All animal experiments were approved by the Baylor College of Medicine Institutional Animal Care and Use Committee (IACUC). All animals used for humanization (including controls) were female, due to fewer postsurgical complications.

### Example 5

### qPCR

Total mRNA was isolated from fresh frozen tissue samples using Purelink RNA mini kit (Invitrogen). 2 μg of total mRNA was reverse transcribed using the qScript cDNA supermix (Quanta Biosciences) and 20ng of cDNA was used for the qPCR reactions, performed with Perfecta SYBR Green Fast Mix (Quanta Biosciences) and analyzed on ABI Prism 7900HT Sequence Detection System (Applied Biosciences). The following primers were used for Por mRNA amplification of PIRF mouse samples:

mPor Fw2
(SEQ ID NO: 14)
GGCCCCACCTGTCAAAGAGAGCAGC
mPor Rev1:
(SEQ ID NO: 15)
CAAACTTGACACCCGTGAGGTCC

For humanized PIRF mouse liver samples, mouse Por and human POR were amplified using the following set of primers:

mPor Fw1:
(SEQ ID NO: 16)
TCTATGGCTCCCAGACGGGAACC
mPor Rev2:
(SEQ ID NO: 17)
CCAATCATAGAAGTCCTGCGCG
hPOR Fw1:
(SEQ ID NO: 18)
CCAATCATAGAAGTCCTGCGCG
hPOR Rev5:
(SEQ ID NO: 19)
ACCTTGGCCGCATCTATGTCGG

Each sample was normalized to Gapdh/GADPH as an internal control gene using the following primers:

mGapdh Fw
(SEQ ID NO: 20)
AGAACATCATCCCTGCATCCA
mGapdh Rev
(SEQ ID NO: 21)
CAGATCCACGACGGACACATT
hGAPDH Fw:
(SEQ ID NO: 22)
CAGAACATCATCCCTGCCTCTAC
hGAPDH Rev:
(SEQ ID NO: 23)
TTGAAGTCAGAGGAGACCACCTG

### Example 6

### RNA-Seq Libraries

Whole-transcriptome RNA sequencing (RNA-Seq) was performed using total RNA extracted from fresh-frozen liver tissue sampled from all seven liver lobes. Total RNA was isolated using the Purelink RNA mini kit (Invitrogen). Libraries were generated from total RNA according to the manufacturer's recommendation using the TrueSeq Stranded mRNA LT kit (Illumina). The libraries were sequenced on a NextSeq 500 sequencer. The average read per sample was 17 millions. RNA-Seq TPM expression values were calculated with RSEM⁵² (version 1.2.17) using the read aligner Bowtie2⁵³ applied to the combined human and mouse NCBI Refseq (3/21/16) transcriptomes. RNA sequencing data is available from European Nucleotide Archive, ENA accession PRJEB14714. Low-abundance cytochromes (human <20 TPM and mouse <20 TPM) were only compared if one of the experimental groups reached >20 TPM. Gene expression has been normalized to three human housekeeping genes and their murine counterparts (PSMB2, PSMB4, RAB7A and VPS2929; Psmb2, Psmb4, Rab7 and Vps29)⁵⁴ RNA-Seq data is available from European Nucleotide Archive, ENA accession code PRJEB14714.

### Example 7

### Western Blot

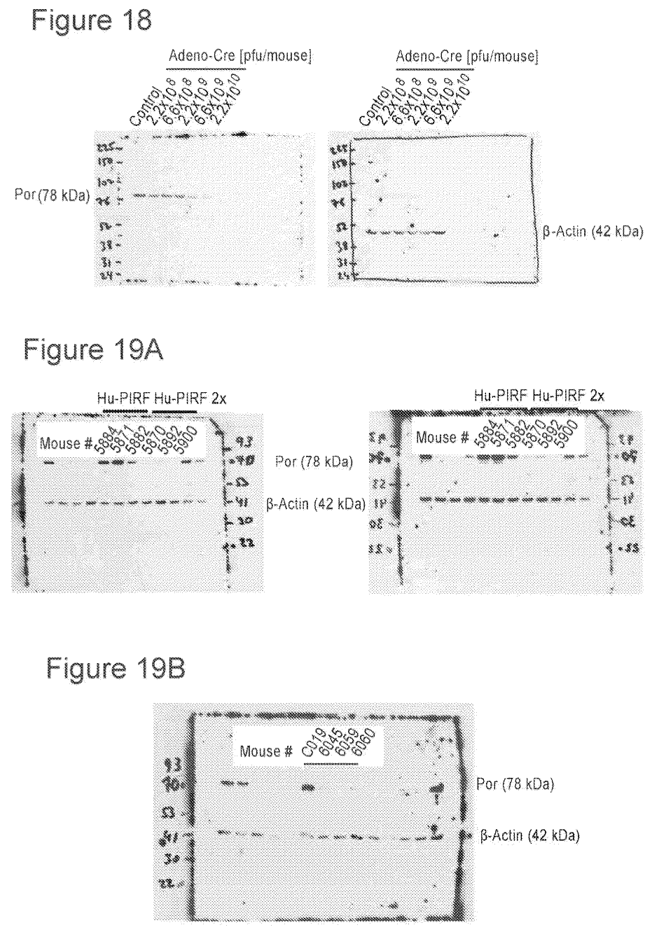

Western blotting was performed as described previously (Bissig-Choisat, B. et al. “Development and rescue of human familial hypercholesterolaemia in a xenograft mouse model.” Nature communications 6, 7339 (2015)). Tissue from snap frozen liver was homogenized in RIPA buffer (Sigma, cat #R0278-50 ml) containing proteases inhibitors (Roche, cat #04693159001). 30 μg of total protein was electrophoresed in a NuPAGE 4-12% Bis Tris Gel (Invitrogen, cat #NP0336BOX) and transferred to a PVDF membrane (Millipore, cat # IPVH00010). The blot was then blocked in 5% milk, followed by primary antibody incubation. Rabbit anti-Por (Abcam cat #ab 13513) or mouse anti-β-actin (Sigma cat #A1978) were diluted 1:1,000 and 1:3,000, respectively (full blots in FIG. 18 and FIG. 19). Secondary antibodies were donkey anti-rabbit IgG/HRP and donkey anti-mouse IgG/HRP (Jackson Immunoresearch Labs, cat #711-035-152 and 711-035-150) used at 1:10,000 and 1:50,000, respectively. The membrane was imaged using Amersham ECL Western Blotting Detection Reagent (General Electric Healthcare Life Sciences, cat #RPN2106).

### Example 8

### Immunohistochemistry

10 μm sections from cryopreserved tissue blocks were fixed with 3% PFA for 15 minutes and incubated overnight at 4° C. with the following primary antibodies: anti-Por (Abcam, cat # ab 13513) diluted 1:500, anti-human Nuclei (EMD Millipore, cat #MAB1281) diluted 1:250 in PBS containing 0.2% Triton X-100 and 0.5% BSA. Secondary antibodies (1:1,000 Alexa-fluor conjugated, Molecular Probes) were incubated for 60 min at room temperature in the same buffer. Sections were mounted with Vectashield plus DAPI (Vector Labs).

### Example 9

### Mouse Husbandry

All mice (6-10 months old, humanized or non-humanized) were maintained under a standard 12-h dark/light cycle with water and chow provided ad libitum. All animal experiments were approved by the Baylor College of Medicine Institutional Animal Care and Use Committee (IACUC).

### Example 10

### Sample Preparation for Mass Spectrometry

One group of mice was treated (i.v.) with gefitinib (10 mg/kg) and housed separately in metabolic cages for 16 h feces collection. Feces samples were weighted and homogenized in water (100 mg feces in 1,000 μl of H₂O). Subsequently, 300 μl of methanol was added to 100 μl of the resulting mixture, followed by centrifugation at 15,000 g for 20 min. The supernatant was transferred to a new Eppendorf vial for a second centrifugation (15,000 g for 20 min). The final concentration of agomelatine is 2 μM. Each supernatant was transferred to an auto sampler vial for analysis (described below).

For atazanavir metabolism in liver, liver samples were harvested 30 min after the treatment of atazanavir (i.v., 30 mg/kg). Briefly, livers were weighted and homogenized in water/MeOH with the internal standard agomelatine [100 mg liver in 300 ul of H₂O/MeOH (v/v 3:1)]. Subsequently, 300 μl of methanol was added to 100 μl of the resulting mixture, followed by centrifugation at 15,000 g for 20 min. The supernatant was transferred to a new Eppendorf vial for a second centrifugation (15,000 g for 20 min). The final concentration of agomelatine is 2 μM in samples. Each supernatant was transferred to an auto sampler vial. Five μl of each prepared sample was injected to a system combining ultra-high performance liquid chromatography (UHPLC) and quadruple time-of-flight mass spectrometry (QTOFMS) for analysis.

### Example 11

### Mass Spectrometry (UHPLC-QTOFMS Analyses)

Metabolites from gefitinib and atazanavir were separated using a 1260 Infinity Binary LC System (Agilent Technologies, Santa Clara, CA) equipped with 100 mm×2.7 mm (Agilent XDB C18) column. The column temperature was maintained at 40° C. The flow rate of was 0.3 mL/min with a gradient ranging from 2% to 98% aqueous acetonitrile containing 0.1% formic acid in a 15-min run. Quadrupole time of flight mass spectrometry (QTOFMS) was operated in positive mode with electrospray ionization. Ultra-highly pure nitrogen was applied as the drying gas (12 L/min) and the collision gas. The drying gas temperature was set at 325° C. and nebulizer pressure was kept at 35 psi. The capillary voltages were set at 3.5 kV. During mass spectrometry, real time mass correction and accurate mass were achieved by continuously measuring standard reference ions at m/z 121.0508, 922.0098 in the positive mode. Mass chromatograms and mass spectra were acquired by MassHunter Workstation data Acquisition software (Agilent, Santa Clara, CA) in centroid and profile formats from m/z 50 to 1000. The acquisition rate was set as 1.5 spectra per second. The method used in this study has been validated by the previous study of gefitinib metabolism in human liver microsomes39. Meanwhile, the quality control samples were performed every 10 samples in the process of the sample running. Due to the authentic compounds of metabolites not available, the metabolite identification was based on their exact mass and MS/MS fragments. The chromatograms and relative abundance of metabolite were performed on Qualitative Analysis software (Agilent, Santa Clara, CA). The relative abundance was evaluated based on integrated peak area of each metabolite.

### Example 12

### Statistics

Sample sizes for experiments were determined by estimated differences between groups and availability of highly humanized mice. No randomization of animals before allocation to experimental groups nor blinding of experimental groups was done. Statistical analysis was performed using PRISM version 6.0 software (Graph Pad software) using Mann-Whitney test, or ANOVA. Statistical significance was assumed with a p-value <0.05 (*). Bars in graphs represent mean +SEM unless noted otherwise. Group size (N) represents biological sample size.

### Example 13

### Generation of Novel Mouse Model for Hepatocyte Repopulation

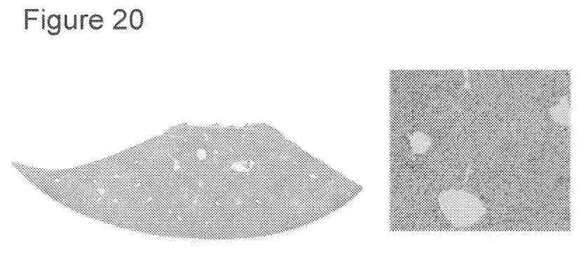

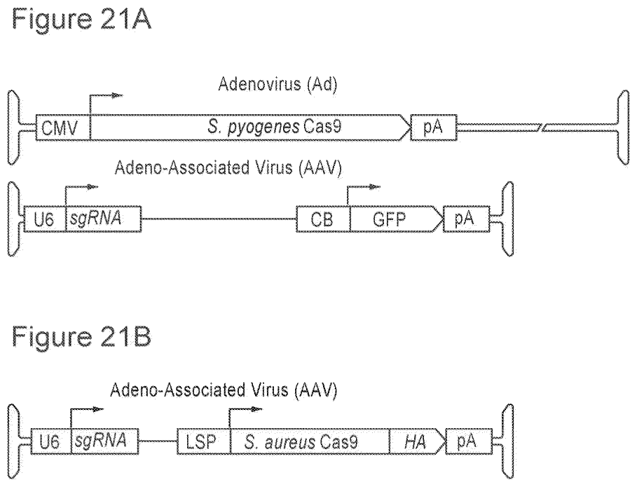

In order to functionally block murine cytochrome metabolism, conditional (floxed exon 3 and 4) knock-out of the NADPH-P450 oxidoreductase (Por) gene by targeting mouse embryonic stem cells 28 was generated (FIG. 4). Injected blastocysts with properly targeted embryonic stem cells produced chimeras with germline transmission of the Por “knock-out first” allele²⁹. Expression from the targeted Por locus using the lacZ expression cassette in the embryo and adult liver was confirmed (FIG. 5). Next mice were bred with a flippase-expressing strain³⁰ to generate a CRE recombinase conditional Por knock-out strain (Por^{c/c}). Homozygous zygotes from this strain were injected with the bacterial type II Clustered Regularly-Interspaced Short Palindromic Repeats/Cas9 (CRISPR-Cas9) system^{31, 32, 33} targeting simultaneous deletion of critical exons of the Il2-rg, Rag2, and Fah genes (FIG. 6 and FIG. 7) to generate the PIRF strain (FIG. 1A). Homozygous PIRF mice are thus immune-deficient, lacking T, B and NK cells, but are healthy and fertile. Since adenoviral gene therapy vectors efficiently transduce hepatocytes in vivo, the Por gene was deleted using an adenovirus coding the CRE recombinase (Adeno-CRE). Increasing doses (2.2×10⁸⁻¹⁰ per mouse) of the virus were injected intravenously into PIRF mice. Quantitative RT-PCR of the Por mRNA in liver revealed efficient deletion only at high doses of adenovirus (FIG. 1B). Immunostaining for Por (FIG. 1C) confirmed these findings, although a minimal residual signal could be detected by Western blotting even at the highest dose used (FIG. 1D). Por-deleted PIRF mouse livers accumulated lipids starting about two weeks after adenoviral transduction (FIG. 2), but in contrast to the immune competent Alb-Cre/Porc/c strain^{25, 27}, without infiltration and lacking necrosis (FIG. 20). Nevertheless, residual Por expressing hepatocytes had a growth advantage over the lipid-rich Por-deleted hepatocytes, and clonal expansion of a few Por expressing cells could be detected four weeks after adenoviral transduction by immunostaining (FIG. 9).

### Example 14

### Characterization of Humanized PIRF Mice

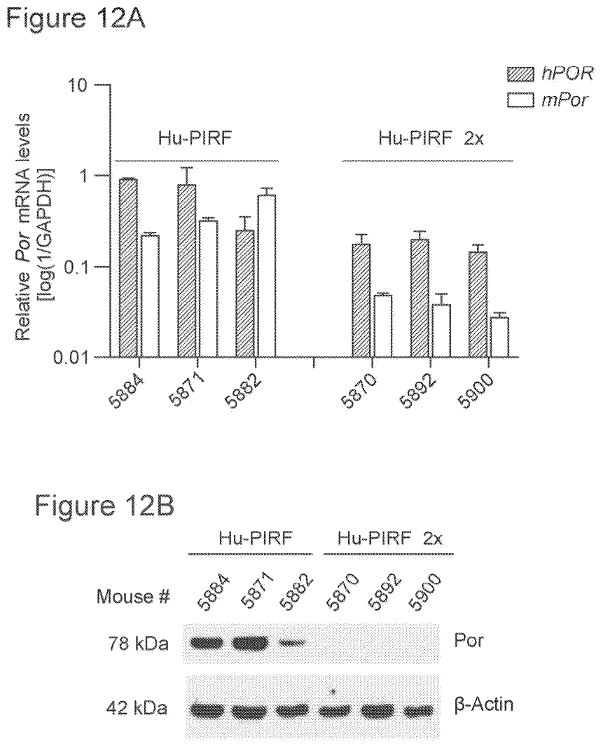

Human liver chimeric mice using the PIRF strain were generated^{5, 20, 34}. To ensure cytochrome P450 metabolism would be human-specific, we injected Adeno-Cre (2.3×10¹⁰ pfu/mouse) before human hepatocyte transplantation and an additional dose of Adeno-Cre in some highly humanized PIRF (Hu-PIRF) mice. Immunostaining revealed that an almost complete deletion of the Por gene could be achieved only in double-injected humanized PIRF (Hu-PIRF 2x) mice (FIG. 2A). Quantitative PCR and Western blotting corroborated the massive reduction of murine Por upon adenoviral delivery of CRE (FIG. 12). Gene expression profiling was performed to compare PIRF mice repopulated with human hepatocytes (Hu-PIRF) that were injected with either Adeno-CRE (Hu-PIRF 2x) or Adeno-GFP (FIG. 2B). Both groups were repopulated with human hepatocytes from the same hepatocyte donors (Table 2) to avoid inter-individual variations.

TABLE 2
Characteristics of human hepatocyte donors used in the present disclosure.

| Hepatocyte | Age* | Gender | Race | BMI | Cause of death | Usage in present study |
|---|---|---|---|---|---|---|
| #1 | 24 | Male | African American | 20.3 | Anoxia | RNAseq (chimeric mice, hepatocytes) |
| #2 | 2 | Female | African American | 19.6 | Head trauma | RNAseq (chimeric mice, hepatocytes) |
| #3 | 45 | Female | Caucasian | 20.8 | Anoxia | RNAseq (Chimeric mice) |
| #4 | 1.2 | Female | Caucasian | 20.8 | Head trauma | Gefetinib metabolites |
| #5 | 18 | Male | Caucasian | 24.3 | Cardiovascular | ATV metabolites |

*in years

Expression of the murine P450 cytochromes was clearly altered for 27 out 38 genes analyzed after Por deletion (FIG. 2C): 24 cytochromes were significantly upregulated (1.5-12.5-fold) and 3 cytochromes significantly downregulated (0.5-0.3-fold). The expression profiles of these murine cytochromes were by in large comparable to those from previous work in non-humanized, Por-deficient mice (Table 1)³⁵. In the human part of the same chimeric liver, human P450 cytochromes were less altered upon deletion of murine Por (FIG. 2D). Half of the human cytochromes were only slightly altered (0.5-1.5-fold change), while the other half were moderately upregulated (1.5-2.4-fold).

Not all human cytochromes serve an important role in xenobiotic metabolism. From the 200 most-prescribed drugs in the United States, about three-quarter are metabolized through P450 cytochromes, of which CYP3A4/5, 2C9, 2C19, 2D6 and 1A2 contribute to ˜95%³⁶. Comparing these human cytochrome clusters from chimeric livers (Hu-PIRF 2x) with the originating, isogenic primary hepatocytes. For this comparison, two donor hepatocytes (Table 2) and the corresponding human (isogenic) liver chimeric mice (N=6). Expression levels were similar for most clusters, and these important cytochromes were all robustly expressed in chimeric livers (FIG. 2E). Interestingly, some human clusters (CYP1A2, CYP2B6, CYP2C19 and CYP3A4) were expressed at even higher levels in the chimeric liver than in primary human hepatocytes.

### Example 15

### Xenobiotic Metabolism of Humanized PIRF Mice

To validate Hu-PIRF mice for human drug metabolism, xenobiotic metabolism of gefitinib³⁷, an inhibitor of epidermal growth factor receptor used against lung cancer and a variety of other neoplasia was used³⁸. Gefitinib is metabolized primarily by the P450 cytochrome system, including CYP3A4 and 2D6.Gefitinib metabolites demonstrate considerable differences between human and mouse liver microsomes³⁹, but regardless of dose, route or species, gefitinib is excreted primarily in the feces (less than 7% in the urine)^{40, 41}. The feces of non-humanized PIRF mice for gefitinib metabolites during the first 24 hours after intravenous injection of gefitinib was then analyzed.

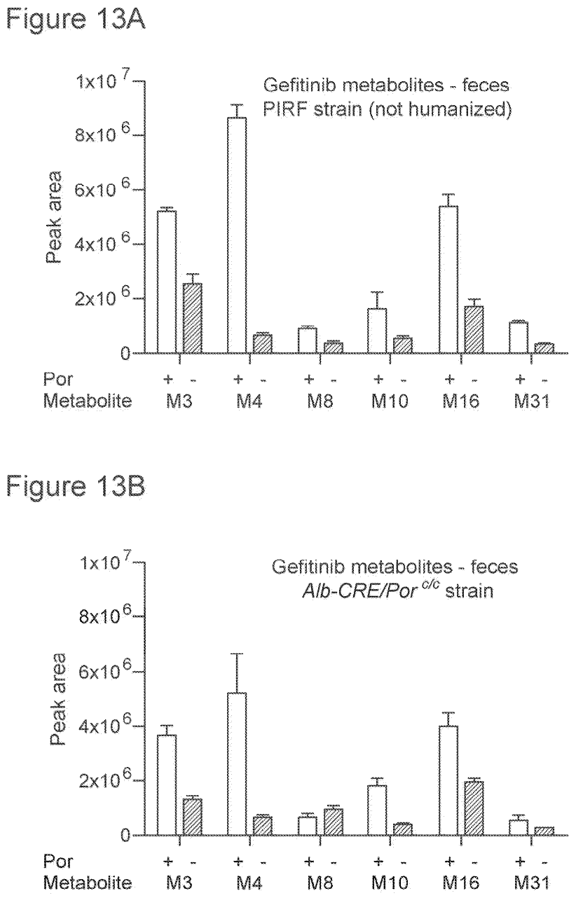

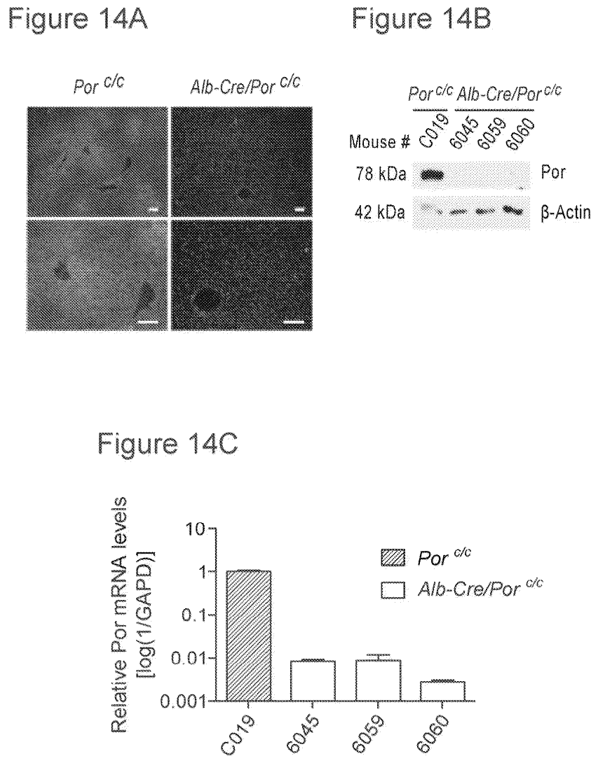

Mass spectrometry revealed a reduction of several gefitinib metabolites upon deletion of the Por gene, implying a Por-dependent P450 cytochrome deficiency for these metabolites (FIG. 3A and FIG. 13A). Since some metabolites were not significantly altered, the possibility that residual Por activity was responsible for persistent murine P450 cytochrome metabolism system was tested. The Porc/c strain was crossed with a transgenic mouse that expresses CRE under the Albumin promoter. Por protein in the liver of Alb-CRE/Porc/c animals was efficiently deleted (FIG. 14); nevertheless, the metabolite profile formed after gefitinib injection was comparable to that of PIRF mice with adenoviral deletion of Por (FIG. 13). This result similarity indicates that gefitinib has both P450-dependent and-independent drug metabolism.

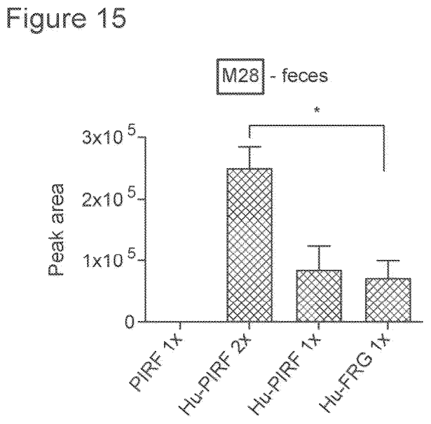

The biggest and most relevant reduction was observed for O-desmethyl gefitinib (M4, M523595), which is by far the most abundant metabolite in human feces. Rodents produce many different metabolites in addition to M4^{40, 41}. (FIG. 3B), so the M4 metabolite in murine Por-deleted and Por-expressing humanized and non-humanized control mice was analyzed (FIG. 10). The highest levels of M4 were detected in murine Por-deficient Hu-PIRF mice, where human hepatocytes preferentially metabolize gefitinib to M4 and the remaining murine hepatocytes are inhibited in their drug metabolism were used (FIG. 3C). Next, to measure other human-specific metabolites. The most abundant human metabolite was M28, which could not be detected at all in non-humanized control mice. Mass spectrometry again showed the highest level of this human-specific metabolite in murine Por-deficient Hu-PIRF mice (FIG. 3D and FIG. 15), confirming that these mice showed liver metabolism more similar to humans.

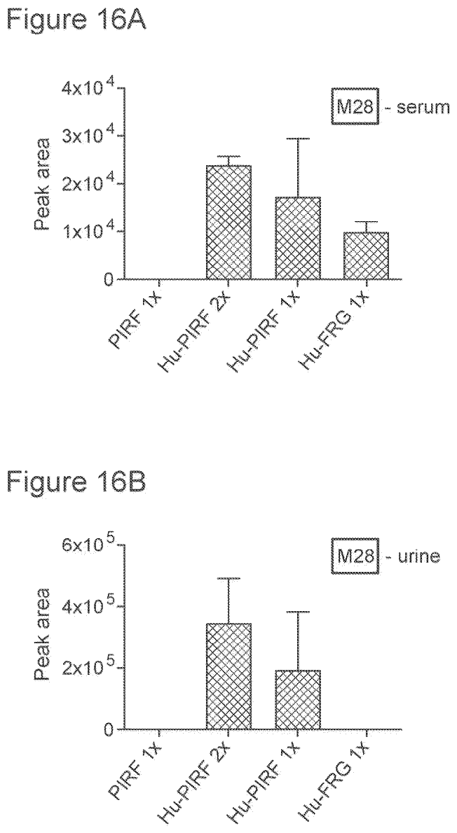

The Por-deficient Hu-PIRF mouse is a novel model system for drug metabolism studies, and therefore was used to analyze different body compartments, e.g. the serum (one hour after injection) and the urine for these key gefitinib metabolites. M4 could not be detected in the urine and was massively reduced (23-fold in Hu-PIRF mice) in the serum, while M28 was detectable at lower concentrations in both the urine and the serum of Hu-PIRF mice (FIG. 16A and FIG. 16B). Although present at lower levels in both compartments, M28 mirrored relative abundance observed in feces (FIG. 3C). These findings confirm that gefitinib metabolites are primarily excreted trough the feces^{40, 41}.

To confirm human xenobiotic metabolism using liver homogenates of PIRF mice. Atazanavir, an antiretroviral drug (protease inhibitor) for treatment of human immunodeficiency virus was tested. Previous studies in human and mouse microsomes demonstrated that atazanavir metabolite M15 is a predominant human metabolite⁴². To determine levels of M15 in humanized PIRF mice, PIRF mice were intravenously injected with atazanavir and their livers harvested, 30 min after injection. M15 levels in Por-deleted humanized PIRF mice were 5.4 times greater than those observed in non-deleted mice (FIG. 3E), again indicating that these mice metabolize drugs as humans do.

### Example 16

### Deletion of the UDP-glucose 6-dehydrogenase (UGDH)

Deletion of the UDP-glucose 6-dehydrogenase (UGDH) leads to depletion of UDP-glucuronate, which is the substrate of all UDP-glucuronosyl transferases (UGT). UGTs glucuronidate lipophilic drugs in the liver (phase II) and thereby contribute to biotransformation of drugs in the liver; glucuronidated drugs are more polar (hydrophilic) and more easily excreted. Deletion of UGDH is embryonically lethal and therefore needs to be deleted conditionally or by somatic genome engineering, similar to POR. Troglitazone was developed as an antidiabetic drug but withdrawn from the market due to hepatotoxicity. Interestingly, mice and humans metabolize the drug differently, meaning that humans mainly generate sulfate metabolites (main circulating metabolites) while glucuronide conjugates of troglitazone are less prevalent in humans. In contrast to mice, which generate mostly glucuronide conjugates. Hence troglitazone offers an opportunity to validate effectiveness of the approach to inhibit UDP-glucuronosyl transferases (UGT) by deletion of UDP-glucose 6-dehydrogenase (UGDH) in human liver chimeric mice in addition to the Por deletion and humanization.

Glutathione synthetase (GSS) catalyzes the second step of glutathione biosynthesis. Glutathione is the substrate of Gluthatione S-transferases (GST), which conjugates the molecule to lipophilic drugs (phase II) and thereby contribute to biotransformation of drugs in the liver.

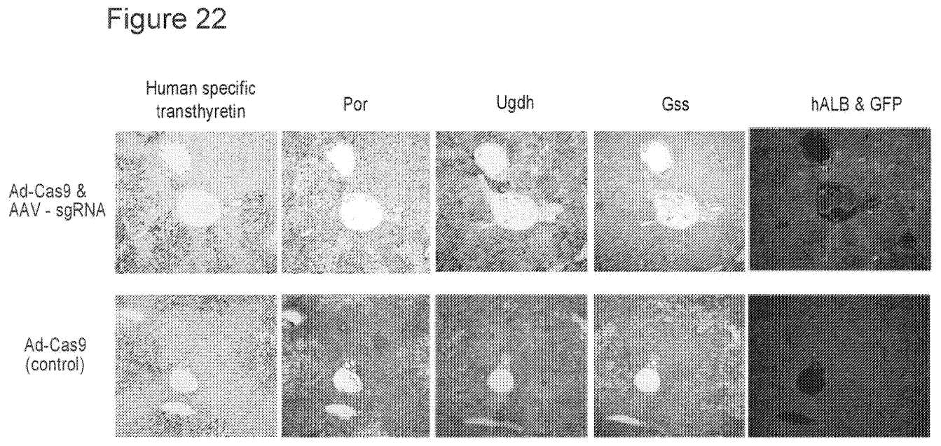

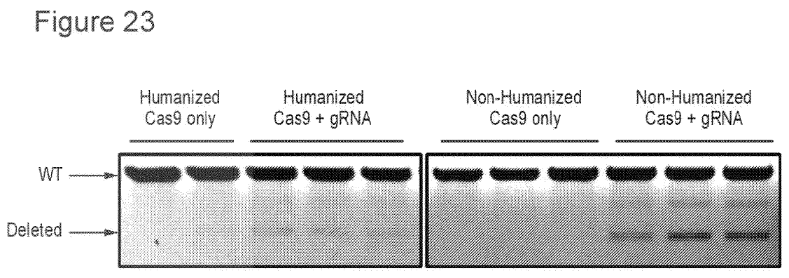

Somatic genome engineering is used to simultaneously delete murine P450 oxidoreductase (Por) and other murine enzymes involved in drug metabolism in humanized mice. Humanized FRG mice (human albumin in murine serum >2 mg/ml) are injected with Adeno-Associated Virus (AAV, serotype 8) expressing sgRNA targeting an early exon of murine Por,UDP-glucose 6-dehydrogenase (Ugdh) or the glutathione synthetase (Gss) gene (see gene therapy vector design, FIG. 21). AAVs are injected (2×10¹¹ GC/AAV/mouse) 1 week before injection of Adenovirus expressing Cas9 (7×10⁹ pfu/Ad/mouse). Control mice are injected with adenoviral vector only (FIG. 22, lower row). Results show a deletion of murine por, as well as ugdh and gss genes. The knockdown by CRISPR/Cas9 of the murine por in humanized mice is substantial but not quite as efficient as the knockdown observed with the loxP/CRE system when looking at the DNA (FIG. 23) and protein levels (FIG. 22). Also, the por deletion particularly in FRG mice is independent of the deletion of the other two genes (ugdh and gss), since their sgRNA (targeting molecule) are all on different AAV vectors. However, immunostaining (FIG. 22) demonstrated that substantial amounts of cells had deletion of por and gss (FIG. 22), while the deletion of ugdh was less efficient.

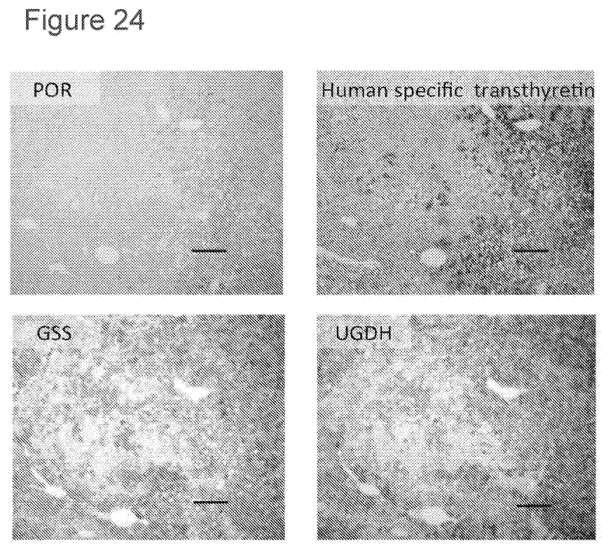

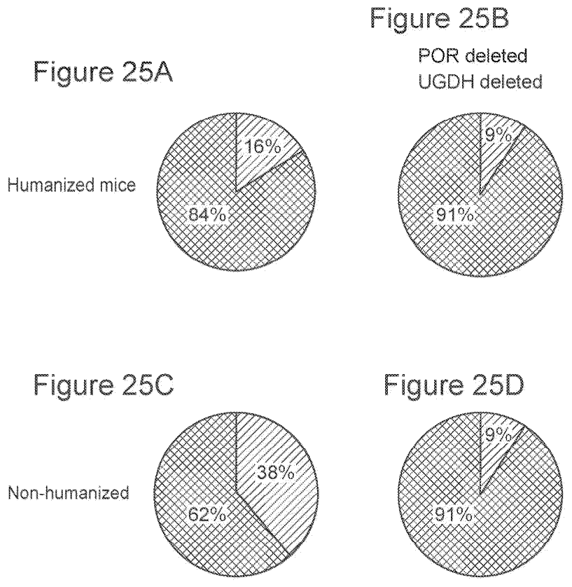

Humanized PIRF mouse with transgenic Alb-CRE and deletion of other murine enzymes involved in drug metabolism are used. Por is deleted by expression of CRE, but instead of adenoviral CRE, this PIRF mouse carries an Alb-CRE sequence within the murine genome. These mice efficiently repopulate with human hepatocytes as evidenced by human specific albumin >2 mg/ml in the murine blood and transthyretin (prealbumin) staining in the chimeric liver (FIG. 24). Murine por is efficiently deleted in the liver of these chimeric mice since the albumin promoter is expressed already in late embryonic stages in the liver. Also in these humanized PIRF mice, in addition to the por, gss and ugdh can also be deleted (FIG. 23).

### Example 17

### Analysis of Troglitazone Metabolites

Troglitazone metabolites (two hours after i.p. injection of 600 mg/kg troglitazone) in the livers of humanized and non-humanized FRG mice with and without Por and Ugdh deletion was analyzed. Non-humanized livers of control mice had much higher amounts of glucuronide conjugates than humans or humanized PIRF mice (FIG. 24). Furthermore, glucuronide conjugates reduced significantly upon deletion of ugdh and por in non-humanized and humanized PIRF mice. This data confirms that deletion of ugdh leads also to a functional impairment or abolishment of UGTs in the human liver chimeric liver.

The present disclosure provides a next generation of humanized mouse model amenable to human drug metabolism with minimal interference from the murine P450 cytochromes. The production of human metabolites for two different drugs between humanized PIRF mice and “normal” humanized FRG mice were compared. Analyses revealed higher concentrations of human metabolites in murine feces and liver homogenate in humanized PIRF mice than in FRG mice and demonstrate that these mice have humanized drug metabolism. The PIRF and FRG strains used in this study are in a mixed (C57B and 129S) genetic background. Aside from potential differences in the background to the two previously published FRG mouse strains^{5,7}, our CRISPR/Cas9 generated knockout strains do not express any transgenes, e.g. the neomycin phosphotransferase that inactivates a wide range of aminoglycoside antibiotics. This model system is useful for early detection of reactive metabolites and is an elegant way to block a large and confounding cluster of drug metabolizing murine enzymes. In addition to the novel mouse model provided herein, the disclosure provides (a) knocking out Por in a combination of multiple organs like the gut and the liver or the lung and the liver would be desirable, (b) additional deletions in other drug-metabolizing enzymes and/or achieving a Por deletion more efficiently. Using transgenic mice expressing Cre recombinase would require yet another crossing step into a quadruple transgenic (PIRF) mouse, however, and an early organ-specific deletion might not generate a robust strain amenable to xenotransplantation.

In summary, the present disclosure provides a novel mouse model combining human chimerism with functional deletion of all murine cytochromes by Por deletion. Such a murine Por-deficient humanization can be used in combination with other repopulation models such as the transgenic uPA mouse^{11,21}. Studies with two different drugs in two different body compartments demonstrate that studies in humanized PIRF mice efficiently identify human metabolites.

### REFERENCES

1. Olson H, et al. Concordance of the toxicity of pharmaceuticals in humans and in animals. Regulatory toxicology and pharmacology: RTP 32, 56-67 (2000).

2. Nelson D R, Zeldin D C, Hoffman S M, Maltais L J, Wain H M, Nebert D W. Comparison of cytochrome P450 (CYP) genes from the mouse and human genomes, including nomenclature recommendations for genes, pseudogenes and alternative-splice variants. Pharmacogenetics 14, 1-18 (2004).

3. Guengerich F P, Cheng Q. Orphans in the human cytochrome P450 superfamily: approaches to discovering functions and relevance in pharmacology. Pharmacological reviews 63, 684-699 (2011).

4. Mercer D F, et al. Hepatitis C virus replication in mice with chimeric human livers. Nat Med 7, 927-933 (2001).

5. Bissig K D, Le T T, Woods N B, Verma I M. Repopulation of adult and neonatal mice with human hepatocytes: a chimeric animal model. Proc Natl Acad Sci USA 104, 20507-20511 (2007).

6. Dandri M, et al. Repopulation of mouse liver with human hepatocytes and in vivo infection with hepatitis B virus. Hepatology 33, 981-988 (2001).

7. Azuma H, et al. Robust expansion of human hepatocytes in Fah(−/−)/Rag2(−/−)/Il2rg(−/−) mice. Nat Biotechnol 25, 903-910 (2007).

8. Hasegawa M, et al. The reconstituted ‘humanized liver’ in TK-NOG mice is mature and functional. Biochemical and biophysical research communications 405, 405-410 (2011).

9. Washburn M L, et al. A humanized mouse model to study hepatitis C virus infection, immune response, and liver disease. Gastroenterology 140, 1334-1344 (2011).

10. Suemizu H, et al. Establishment of a humanized model of liver using NOD/Shi-scid IL2Rgnull mice. Biochem Biophys Res Commun 377, 248-252 (2008).

11. Heckel J L, Sandgren E P, Degen J L, Palmiter R D, Brinster R L. Neonatal bleeding in transgenic mice expressing urokinase-type plasminogen activator. Cell 62, 447-456 (1990).

12. Tateno C, et al. Near completely humanized liver in mice shows human-type metabolic responses to drugs. Am J Pathol 165, 901-912 (2004).

13. Samuelsson K, et al. Troglitazone metabolism and transporter effects in chimeric mice: a comparison between chimeric humanized and chimeric murinized FRG mice. Xenobiotica; the fate of foreign compounds in biological systems 44, 186-195 (2014).

14. Lootens L, et al. Steroid metabolism in chimeric mice with humanized liver. Drug testing and analysis 1, 531-537 (2009).

15. Foster JR, et al. Differential effect of troglitazone on the human bile acid transporters, MRP2 and BSEP, in the PXB hepatic chimeric mouse. Toxicologic pathology 40, 1106-1116 (2012).

16. Xu D, et al. Fialuridine induces acute liver failure in chimeric TK-NOG mice: a model for detecting hepatic drug toxicity prior to human testing. PLOS medicine 11, e1001628 (2014).

17. Bateman T J, Reddy V G, Kakuni M, Morikawa Y, Kumar S. Application of chimeric mice with humanized liver for study of human-specific drug metabolism. Drug Metab Dispos 42, 1055-1065 (2014).

18. Nishimura T, et al. Using chimeric mice with humanized livers to predict human drug metabolism and a drug-drug interaction. J Pharmacol Exp Ther 344, 388-396 (2013).

19. Tanoue C, et al. Prediction of human metabolism of the sedative-hypnotic zaleplon using chimeric mice transplanted with human hepatocytes. Xenobiotica; the fate of foreign compounds in biological systems 43, 956-962 (2013).

20. Bissig K D, et al. Human liver chimeric mice provide a model for hepatitis B and C virus infection and treatment. The Journal of clinical investigation 120, 924-930 (2010).

21. Meuleman P, et al. Morphological and biochemical characterization of a human liver in a uPA-SCID mouse chimera. Hepatology 41, 847-856 (2005).

22. Kato K, et al. Development of Murine Cyp3a Knockout Chimeric Mice with Humanized Liver. Drug Metab Dispos 43, 1208-1217 (2015).

23. Nakada N, et al. Murine Cyp3a knockout chimeric mice with humanized liver: prediction of the metabolic profile of nefazodone in humans. Biopharmaceutics & drug disposition, (2015).

24. Shen A L, O'Leary K A, Kasper C B. Association of multiple developmental defects and embryonic lethality with loss of microsomal NADPH-cytochrome P450 oxidoreductase. J Biol Chem 277, 6536-6541 (2002).

25. Wu L, et al. Conditional knockout of the mouse NADPH-cytochrome p450 reductase gene. Genesis 36, 177-181 (2003).

26. Henderson C J, et al. Inactivation of the hepatic cytochrome P450 system by conditional deletion of hepatic cytochrome P450 reductase. J Biol Chem 278, 13480-13486 (2003).

27. Gu J, et al. Liver-specific deletion of the NADPH-cytochrome P450 reductase gene: impact on plasma cholesterol homeostasis and the function and regulation of microsomal cytochrome P450 and heme oxygenase. J Biol Chem 278, 25895-25901 (2003).

28. Thomas K R, Capecchi M R. Site-directed mutagenesis by gene targeting in mouse embryo-derived stem cells. Cell 51, 503-512 (1987).

29. Skarnes W C, et al. A conditional knockout resource for the genome-wide study of mouse gene function. Nature 474, 337-342 (2011).

30. Farley F W, Soriano P, Steffen L S, Dymecki S M. Widespread recombinase expression using FLPeR (flipper) mice. Genesis 28, 106-110 (2000).

31. Haft D H, Selengut J, Mongodin E F, Nelson K E. A guild of 45 CRISPR-associated (Cas) protein families and multiple CRISPR/Cas subtypes exist in prokaryotic genomes. PLOS computational biology 1, e60 (2005).

32. Jinek M, Chylinski K, Fonfara I, Hauer M, Doudna J A, Charpentier E. A programmable dual-RNA-guided DNA endonuclease in adaptive bacterial immunity. Science 337, 816-821 (2012).

33. Jansen R, Embden J D, Gaastra W, Schouls L M. Identification of genes that are associated with DNA repeats in prokaryotes. Molecular microbiology 43, 1565-1575 (2002).

34. Bissig-Choisat B, et al. Development and rescue of human familial hypercholesterolaemia in a xenograft mouse model. Nature communications 6, 7339 (2015).

35. Weng Y, DiRusso C C, Reilly A A, Black P N, Ding X. Hepatic gene expression changes in mouse models with liver-specific deletion or global suppression of the NADPH-cytochrome P450 reductase gene. Mechanistic implications for the regulation of microsomal cytochrome P450 and the fatty liver phenotype. J Biol Chem 280, 31686-31698 (2005).

36. Williams J A, et al. Drug-drug interactions for UDP-glucuronosyltransferase substrates: a pharmacokinetic explanation for typically observed low exposure (AUCi/AUC) ratios. Drug Metab Dispos 32, 1201-1208 (2004).

37. Barker A J, et al. Studies leading to the identification of ZD1839 (IRESSA): an orally active, selective epidermal growth factor receptor tyrosine kinase inhibitor targeted to the treatment of cancer. Bioorganic & medicinal chemistry letters 11, 1911-1914 (2001).

38. Herbst R S, Fukuoka M, Baselga J. Gefitinib—a novel targeted approach to treating cancer. Nat Rev Cancer 4, 956-965 (2004).

39. Liu X, et al. Metabolomics reveals the formation of aldehydes and iminium in gefitinib metabolism. Biochem Pharmacol 97, 111-121 (2015).

40. McKillop D, et al. Metabolic disposition of gefitinib, an epidermal growth factor receptor tyrosine kinase inhibitor, in rat, dog and man. Xenobiotica; the fate of foreign compounds in biological systems 34, 917-934 (2004).

41. Scheffler M, Di Gion P, Doroshyenko O, Wolf J, Fuhr U. Clinical pharmacokinetics of tyrosine kinase inhibitors: focus on 4-anilinoquinazolines. Clinical pharmacokinetics 50, 371-403 (2011).

42. Li F, Lu J, Wang L, Ma X. CYP3A-mediated generation of aldehyde and hydrazine in atazanavir metabolism. Drug Metab Dispos 39, 394-401 (2011).

43. Baillie T A. Future of toxicology-metabolic activation and drug design: challenges and opportunities in chemical toxicology. Chemical research in toxicology 19, 889-893 (2006).

44. Guengerich F P, MacDonald J S. Applying mechanisms of chemical toxicity to predict drug safety. Chemical research in toxicology 20, 344-369 (2007).

45. Dalvie D, et al. Assessment of three human in vitro systems in the generation of major human excretory and circulating metabolites. Chemical research in toxicology 22, 357-368 (2009).

46. Anderson S, Luffer-Atlas D, Knadler M P. Predicting circulating human metabolites: how good are we? Chemical research in toxicology 22, 243-256 (2009).

47. Pettitt S J, et al. Agouti C57BL/6N embryonic stem cells for mouse genetic resources. Nat Methods 6, 493-495 (2009).

48. Cradick T J, Qiu P, Lee C M, Fine E J, Bao G. COSMID: A Web-based Tool for Identifying and Validating CRISPR/Cas Off-target Sites. Molecular therapy Nucleic acids 3, e214 (2014).

49. Hwang W Y, et al. Efficient genome editing in zebrafish using a CRISPR-Cas system. Nat Biotechnol 31, 227-229 (2013).

50. Cong L, et al. Multiplex genome engineering using CRISPR/Cas systems. Science 339, 819-823 (2013).

51. Ponder K P, et al. Mouse hepatocytes migrate to liver parenchyma and function indefinitely after intrasplenic transplantation. Proc Natl Acad Sci USA 88, 1217-1221 (1991).

52. Li B, Dewey C N. RSEM: accurate transcript quantification from RNA-Seq data with or without a reference genome. BMC Bioinformatics 12, 323 (2011).

53. Langmead B, Salzberg S L. Fast gapped-read alignment with Bowtie 2. Nat Methods 9, 357-359 (2012).

54. Eisenberg E, Levanon E Y. Human housekeeping genes, revisited. Trends in genetics: TIG 29, 569-574 (2013).

What is claimed is:

1. A method for preparing a chimeric non-human animal comprising human hepatocytes, the method comprising:
(a) providing a non-human animal comprising a reduction or deletion of NADPH-P450 oxidoreductase (Por) gene resulting in reduced or absent expression of Por protein; and
(b) transplanting human hepatocytes into the non-human animal.

2. The method of claim 1, wherein providing the non-human animal comprises reducing or deleting the Por gene resulting in reduced or absent expression of Por protein.

3. The method of claim 1, wherein the reduced or deleted Por gene is a conditional knockdown or knockout of the Por gene.

4. The method of claim 1, wherein the reduced or deleted Por gene is the result of a mutation, a transgene, treatment with an exogenous substance or somatic genome engineering.

5. The method of claim 4, wherein the somatic genome engineering comprises Guide RNA (gRNA) and Caspase 9.

6. The method of claim 1, wherein the non-human animal comprises a floxed allele of the Por gene, and wherein the non-human animal is provided with a Cre recombinase sufficient to produce a conditional knockout of the Por gene.

7. The method of claim 6, wherein the non-human animal comprising the floxed allele of the Por gene is provided with at least a first dose of a virus that encodes Cre recombinase.

8. The method of claim 7, wherein the non-human animal is provided with at least a second dose of a virus that encodes Cre recombinase.

9. The method of claim 1, comprising
(a) providing a non-human animal comprising a floxed allele of the Por gene with a first does of a virus that encodes Cre recombinase;
(b) transplanting human hepatocytes into the non-human animal; and
(c) providing the non-human animal with a second dose of a virus that encodes Cre recombinase.

10. The method of claim 9, wherein steps (a) and (b) occur sequentially.

11. The method of claim 9, wherein steps (a) and (b) occur simultaneously.

12. The method of claim 7, wherein the non-human animal comprising the floxed allele of the Por gene is crossed with a transgenic non-human animal strain expressing Cre recombinase.

13. The method of claim 1, wherein the non-human animal further comprises a reduction or deletion of at least one additional gene encoding an enzyme involved in drug metabolism.

14. The method of claim 13, wherein the at least one additional enzyme is a phase II drug enzyme.

15. The method of claim 1, wherein the non-human animal further comprises a reduction or deletion of UDP-glucose 6-dehydrogenase (UGDH) gene.

16. The method of claim 1, wherein the non-human animal further comprises a reduction or deletion of Glutathione synthetase (GSS) gene.

17. The method of claim 1, wherein the non-human animal is selected from the group consisting of primate, bird, mouse, rat, fowl, dog, cat, cow, horse, goat, camel, sheep and pig.

18. The method of claim 1, wherein the non-human animal is a mouse.

19. The method of claim 1, wherein the non-human animal is selected from the group consisting of (i) the FRG (Fah^{-/-}/Rag2^{-/-}/Il2rg^{-/-}) non-human animal, (ii) a transgenic urokinase type plasminogen activator (uPA) non-human animal, which overexpress uPA under an inducible promoter, preferably a liver-restricted albumin promoter, (iii) the thymidine kinase-NOD/Shi-scid/IL-2Rγ^{null} (TK-NOG) non-human animal, which is a immunodeficient NOG non-human animal with transgenic expression of thymidine kinase under control of liver-restricted promoter, (iv) a non-human animal expressing an inducible Caspase 8 in the liver, and (v) a non-human animal expressing an inducible Caspase 9 in the liver.

20. A chimeric non-human animal, offspring thereof, or a portion thereof, which has a chimeric liver comprising human hepatocytes, prepared by the method according to any one of claims 1-19.

21. The chimeric non-human animal of claim 20, wherein the non-human animal is selected from the group consisting of primate, bird, mouse, rat, fowl, dog, cat, cow, horse, goat, camel, sheep and pig.

22. The chimeric non-human animal of claim 20, wherein the non-human animal is a mouse.

23. The chimeric non-human animal of claim 20, wherein chimeric non-human animal substantially lacks autogenous hepatocytes.

24. The chimeric non-human animal of claim 20, wherein human hepatocytes account for at least 60% of all hepatocytes in the chimeric liver.

25. The chimeric non-human animal of claim 20, wherein human hepatocytes account for at least 70% of all hepatocytes in the chimeric liver.

26. The chimeric non-human animal of claim 20, wherein human hepatocytes account for at least 80% of all hepatocytes in the chimeric liver.

27. The chimeric non-human animal of claim 20, wherein human hepatocytes account for at least 90% of all hepatocytes in the chimeric liver.

28. The chimeric non-human animal of claim 20, wherein the chimeric non-human animal is immunodeficient.

29. A method for screening for a substance that affects human liver functions, comprising:
(a) administering a test substance to the chimeric non-human animal of claim 20;
(b) measuring one or more values in the chimeric non-human animal to which the test substance is administered in (a); and
(c) selecting a test substance that causes an increase or decrease in one or more values measured in (b), compared with the one or more values measured in a chimeric non-human animal to which no test substance is administered.

30. The method of claim 29, wherein the one or more values is selected from the group consisting of a metabolite of the test substance, human albumin concentration, body weight curve, liver-weight-to-body-weight ratio, total albumin level, total protein level, Alanine Aminotransferase (ALT) level, Aspartate Aminotransferase (AST) level, and total bilirubin level, creatinine, Blood Urea Nitrogen (BUN), troponine, blood count, TSH and histological assessment for pathologies in the human and non-human organs.

31. A method for evaluating the toxicity of a test substance against human hepatocytes and any other non-human organ in the non-human chimeric animal, comprising:
(a) administering a test substance to the chimeric non-human animal of claim 20;
(b) measuring one or more indicators in the chimeric non-human animal to which the test substance is administered in (a); and
(c) evaluating the effect of the test substance on human hepatocytes using, one or more indicators measured in (b), compared with the one or more indicators measured in a chimeric non-human animal to which no test substance is administered.

32. The method of claim 31, wherein the one or more indicators is selected from the group consisting of an increase or a decrease in any one or more of a metabolite of the test substance, human albumin concentration, body weight curve, liver-weight-to-body-weight ratio, total albumin level, total protein level, ALT level, AST level, and total bilirubin level, creatinine, BUN, troponine, blood count and TSH as well as histological alterations including but not limited to necrosis and apoptosis in the human and non-human organs.
